(19) United States
(12) Patent Application Publication
(10) Pub. No.: US 2025/0281062 A1
(43) Pub. Date: Sep. 11, 2025

(54) SYSTEM AND METHOD FOR NON-CONTACT PEOPLE LOCALIZATION AND VITAL SIGNS MONITORING VIA FMCW RADAR

(71) Applicant: YEDA RESEARCH AND DEVELOPMENT CO. LTD., Rehovot (IL)

(72) Inventors: Yonina ELDAR, Rehovot (IL); Yonathan EDER, Rehovot (IL)

(21) Appl. No.: 19/220,408

(22) Filed: May 28, 2025

Related U.S. Application Data
(63) Continuation-in-part of application No. PCT/IL2023/050461, filed on May 7, 2023.
(60) Provisional application No. 63/386,621, filed on Dec. 8, 2022.
(60) Provisional application No. 63/742,519, filed on Jan. 7, 2025.

(51) Int. Cl. A61B 5/05; A61B 5/0205; A61B 5/024; A61B 5/08; G01S 7/41; G01S 13/536; G16H 40/67

(52) U.S. Cl. CPC A61B 5/05; A61B 5/0205; G01S 7/415; G01S 13/536; G16H 40/67; A61B 5/024; A61B 5/0816

(57) ABSTRACT

A monitoring system is presented for monitoring vital signs of subject(s). The monitoring system includes a control system configured for signal communication with a frequency modulated continuous wave (FMCW) radar to process measured data which is received from a single-channel front end of receiver of said FMCW radar and which is in the form of data matrix indicative of consecutive beat signals. The control system comprises a data processing utility comprising: a localization module configured and operable to process the measured data indicative of said data matrix and provide support recovery data indicative of the received signals originated at localized one or more subjects; and a vital signs monitoring module configured and operable to analyze the support recovery data and monitor vital signs of said localized one or more subjects.

### CROSS-REFERENCE TO RELATED APPLICATIONS

This application is a continuation-in-part of International Patent Application No. PCT/IL2023/050461, filed on May 7, 2023, which claims the priority benefit under 35 U.S.C. § 119 of U.S. Provisional Patent Application No. 63/386,621, filed on Dec. 8, 2022, and also claims the priority benefit under 35 U.S.C. § 119 of U.S. Provisional Patent Application No. 63/742,519, filed on Jan. 7, 2025, the contents of which are hereby incorporated in their entireties by reference.

### TECHNOLOGICAL FIELD

The present invention is in the general field of non-contact people localization and vital signs monitoring via radar.

### BACKGROUND ART

References considered to be relevant as background to the presently disclosed subject matter are listed below:
1. P. Ceballos-Vásquez, G. Rolo-González, E. Hérnandez-Fernaud, D. Díaz-Cabrera, T. Paravic-Klijn, and M. Burgos-Moreno, “Psychosocial factors and mental work load: a reality perceived by nurses in intensive care units1,” Revista latino-americana de enfermagem, vol. 23, pp. 315-322, 2015.
2. A. A. Almenyan, A. Albuduh, and F. Al-Abbas, “Effect of NursingWorkload in Intensive Care Units,” Cureus, vol. 13, no. 1, 2021.
3. F. Fioranelli, J. Le Kernec, and S. Shah, “Radar for health care: Recognizing human activities and monitoring vital signs,” IEEE Potentials, vol. 38, no. 4, pp. 16-23, 2019.
4. C. Gu, Y. He, and J. Zhu, “Noncontact vital sensing with a miniaturized 2.4 G[Hz] circularly polarized Doppler radar,” IEEE Sensors Letters, vol. 3, no. 7, pp. 1-4, 2019.
5. Zhao, Heng, et al., “A noncontact breathing disorder recognition system using 2.4-GHz digital-IF Doppler radar,” IEEE journal of biomedical and health informatics, vol. 23, no. 1, pp. 208-217, 2018.
6. T. K. Vodai, K. Oleksak, T. Kvelashvili, F. Foroughian, C. Bauder, P. Theilman, A. Fathy, and O. Kilic, “Enhancement of Remote Vital Sign Monitoring Detection Accuracy Using Multiple-Input Multiple-Output 77 G[Hz] FMCW Radar,” IEEE Journal of Electromagnetics, RF and Microwaves in Medicine and Biology, 2021.
7. A. Ahmad, J. Roh, D. Wang, and A. Dubey, “Vital signs monitoring of multiple people using a FMCW millimeter-wave sensor,” In 2018 IEEE Radar Conference (RadarConf18), pp. 1450-1455, 2018.
8. F. Adib, H. Mao, Z. Kabelac, D. Katabi, and R. C. Miller, “Smart homes that monitor breathing and heart rate,” In Proceedings of the 33rd annual ACM conference on human factors in computing systems, pp. 837-846, 2015.
9. M. Mercuri, I. Lorato, Y. H. Liu, F. Wieringa, C. Van Hoof, and T. Torfs, “Vital-sign monitoring and spatial tracking of multiple people using a Non-contact radar-based sensor,” Nature Electronics, vol. 2, no. 6, pp. 252-262, 2019.
10. G. Sacco, E. Piuzzi, E. Pittella, and S. Pisa, “An FMCW radar for localization and vital signs measurement for different chest orientations,” Sensors, vol. 20, no. 12, pp. 3489, 2020.
11. M. Alizadeh, G. Shaker, J. De Almeida, P. Morita, and S. Safavi-Naeini, “Remote monitoring of human vital signs using mm-wave FMCW radar,” IEEE Access, vol. 7, pp. 54958-54968, 2019.
12. E. Antolinos, F. García-Rial, C. Hernández, D. Montesano, J. I. Godino Llorente, and J. Grajal, “Cardiopulmonary Activity Monitoring Using Millimeter Wave Radars,” Remote Sensing, vol. 12, no. 14, pp. 2265, 2020.
13. Y. Wang, W. Wang, M. Zhou, A. Ren, and Z. Tian, “Remote monitoring of human vital signs based on 77-G[Hz] mm-wave FMCW radar,” Sensors, vol. 20, no. 10, pp. 2999, 2020.
14. H. Lee, B. H. Kim, J. K. Park, J. G Yook, “A novel vital-sign sensing algorithm for multiple subjects based on 24-G[Hz] FMCW Doppler radar,” Remote Sensing, vol. 11, no. 10, pp. 12-37, 2019.
15. E. Turppa, J. Kortelainen, O. Antropov, and T. Kiuru, “Vital sign monitoring using FMCW radar in various sleeping scenarios,” Sensors, vol. 20, no. 22, pp. 6505, 2020.
16. S. Kim, and K. K. Lee, “Low-complexity joint extrapolation-MUSIC-based 2-D parameter estimator for vital FMCW radar,” IEEE sensors journal, vol. 19, no. 6, pp. 2205-2216, 2018.
17. R. Mahendra, S. K. Mohammed, and R. K. Mallik, ‘Compensation of Receiver IQ Imbalance in mm-wave Hybrid Beamforming Systems,” In 2020 IEEE 92nd Vehicular Technology Conference, pp. 1-6, 2020.
18. U. Rossman, R. Tenne, O. Solomon, I. Kaplan-Ashiri, T. Dadosh, Y. C. Eldar, and, D. Oron, “Rapid quantum image scanning microscopy by joint sparse reconstruction,” Optica, vol. 6, no. 10, pp. 1290-1296, 2019.
19. S. Schellenberger et al., “A dataset of clinically recorded radar vital signs with synchronised reference sensor signals,” Scientific data, vol. 7, no. 291, pp. 1-11, 2020.
20. J. Liu, Y. Li, and C. Gu, Non-contact Vital Signs Monitoring, in Academic Press, 2022, chapter 9-Radar-based vital signs monitoring, pp. 181-203.
21. Orchisama Das, Jonathan S. Abel, Julius O. Smith III, “Fast MUSIC-an Efficient Implementation of The MUSIC Algorithm for Frequency Estimation of Approximately Periodic Signals,” In Proceedings of the 21st International Conference on Digital Audio Effects (DAFx-18), Aveiro, Portugal, 2018.
22. D. Sasakawa, N. Honma, T. Nakayama and S. Iizuka, “Fast Living-Body Localization Algorithm for MIMO Radar in Multipath Environment,” in IEEE Transactions on Antennas and Propagation, vol. 66, no. 12, pp. 7273-7281 December 2018, doi: 10.1109/TAP.2018.2870405.
23. K. Konno, M. Nango, N. Honma, K. Nishimori, N. Takemura and T. Mitsui, “Experimental evaluation of estimating living body direction using array antenna for multipath environment,” IEEE Antennas and Wireless Propagation Letters, vol. 13, pp. 718-721, 2014.
24. Y. Hu and T. Toda, “Remote vital signs measurement of indoor walking persons using mm-wave FMCW radar,” IEEE Access, vol. 10, pp. 78 219-78 230, 2022.
25. Y. Rong, K. V. Mishra, and D. W. Bliss, “Multiple moving targets heartbeat estimation and recovery using multi-frequency radars,” in 2021 IEEE Radar Conference (RadarConf21). IEEE, 2021, pp. 1-5.
26. K. Han and S. Hong, “Cough detection using millimeter-wave FMCW radar,” in ICASSP 2023-2023 IEEE International Conference on Acoustics, Speech and Signal Processing (ICASSP). IEEE, 2023, pp. 1-5.
27. X. Li, X. Wang, Q. Yang, and S. Fu, “Signal processing for TDM MIMO FMCW millimeter-wave radar sensors,” IEEE Access, vol. 9, pp. 167959-167971, 2021.
28. A. Jalil, H. Yousaf, and M. I. Baig, “Analysis of CFAR techniques,” in 2016 13th International Bhurban Conference on Applied Sciences and Technology (IBCAST). IEEE, 2016, pp. 654-659.
29. J. Xiong, H. Hong, L. Xiao, E. Wang, and X. Zhu, “Vital signs detection with difference beamforming and orthogonal projection filter based on SIMO-FMCW radar,” IEEE Transactions on Microwave Theory and Techniques, vol. 71, no. 1, pp. 83-92, 2022.
30. Y. Wang, Y. Shui, X. Yang, Z. Li, and W. Wang, “Multi-target vital signs detection using frequency-modulated continuous wave radar,” EURASIP Journal on Advances in Signal Processing, vol. 2021, pp. 1-19, 2021.
31. F. Wang, X. Zeng, C. Wu, B. Wang, and K. R. Liu, “Driver vital signs monitoring using millimeter wave radio,” IEEE Internet of Things Journal, vol. 9, no. 13, pp. 11283-11298, 2021.

Acknowledgement of the above references herein is not to be inferred as meaning that these are in any way relevant to the patentability of the presently disclosed subject matter.

### BACKGROUND

In the last decade, the rise in chronic health conditions alongside the increase in the elderly population, has resulted in a growing need for health care approaches that emphasize long-term monitoring in addition to urgent intervention. Monitoring of human vital signs, however, entails numerous difficulties. First, current monitoring devices are typically in physical contact with the measured body, therefore may lead to irritation or general discomfort for the patient and can be easily detached. Second, usually monitoring devices are connected to patients by medical staff, whether in clinics or hospitals, by a time-consuming interaction that increases the risk of infections and disease transmission, especially during times of pandemics, such as COVID-19. In addition, the manner of connection greatly affects the results, thus it requires considerable skill and experience. Beyond that, many medical teams suffer from high workloads which ultimately lead to an increase in mortality, infections and duration of hospitalization, e.g., in intensive care units [1]-[2].

Remote sensing technology such as radar systems can be ideal in these situations since they do not require users to wear, carry, or interact with any additional electronic device [3]. In recent years, several works addressed this issue, attempting to remotely monitor human vital signs such as respiration rate (RR) and heart rate (HR), using radars [4]-[16]. Initially, the family of continuous wave (CW) radars was proposed as simple and reliable devices for remote measurement of cardiac-related chest movements and respiratory activity [4]-[5], having the advantages of low transmission power and high sensitivity. However, they do not provide patient distance information from the radar, nor can they separate returns from different objects. To overcome this limitation, many works have turned to using frequency modulated continuous wave (FMCW) radars [6]-[16].

### GENERAL DESCRIPTION

The present disclosure provides a novel technique for non-contact monitoring of vital signs of multiple subjects (stationary or moving subjects) using mm-wave electromagnetic radiation, in particular FMCW radar.

FMCW technology allows spatial separation and potentially monitoring of several people simultaneously, which can reduce both loads and financial costs. However, accurate simultaneous extraction of multiple-people's cardiopulmonary activity using FMCW radars, is still a challenge in terms of performance, and currently lacks adequate mathematical modelling, leading to sub-optimal solutions.

The conventional algorithmic framework for non-contact vital signs monitoring (NCVSM) of multiple people using FMCW radars, is a recurring process in which an estimate of human vital signs is evaluated and recorded at each fixed time interval.

In each iteration, a processing matrix is compiled from the raw data obtained by both the In-phase (I) and Quadrature (Q) channels of the radar, through which the samples attributed to human cardiopulmonary activity are located and extracted. Given estimated human thoracic vibrations, various techniques can be used to monitor each human's desired RR and HR.

Traditionally, at each monitoring repetition, both the I and Q channels are used to assemble a convenient complex matrix for further processing [6]-[16]. The main drawbacks of using both the I and Q channels are the lack of perfect orthogonality, and a difference in gain levels in each channel, namely the I/Q imbalance limitation [17], which may corrupt the desired information to be extracted. This imbalance can be compensated for by methods such as the Gram-Schmidt orthogonalization procedure (GSOP), but there is no guarantee for optimal corrections in realistic noisy environments.

Once the complex map is assembled, a human localization procedure is performed based on a transformed version of the map. Mercuri et al. [9] performed manual localization based on the intensities of the map, knowing in advance the number of people to be monitored, and their true distance from the radar. Alizadeh et al. selected the range bin with the maximal average power. In real-world scenarios, information about the number of people is not typically available, thus relying solely on spectral magnitudes, and can produce erroneous decisions due to strong signal reflections obtained from static objects in the radar's field of view (FOV).

Several works tried to circumvent the effect of clutter on human localization for NCVSM. Adib et al. [8] subtracted consecutive time measurements to eliminate reflections off static objects. In [10], the amplitude of a padded fast Fourier transform (FFT) collected over one frame period was compared to the standard deviation based std estimate, in a room containing furniture. Antolinos et al. performed a clipping procedure on a zero-padded FFT map to isolate the target from interfering objects. However, these methods lack adequate theoretical explanations and may be sensitive to vibrating clutter, such as fans.

Once the human-related vectors have been correctly located, the thoracic vibration pattern of each individual is extracted. The most commonly used methods to estimate the considered vital signs from the extracted pattern are based on the discrete Fourier transform (DFT) spectrum, utilizing the property that, in resting state, the frequency bands of heartbeat and respiration do not overlap [9]-[12]. Despite all the well-known benefits of DFT-spectrum analysis, it presents limitations for the considered problem in terms of both resolution and signal representation which ultimately impairs estimation performance.

The technique of the present disclosure provides a novel vital signs monitoring system using radar radiation, which utilizes a novel approach for localization of subjects in a region of interest (radially and azimuthally with respect to the radar) in a clutter-rich environment and/or monitoring the vital signs of the localized subjects.

The technique of the present disclosure can be implemented by a radar system of any configuration, utilizing any suitable antenna setup, including one or more transmitters and one or more receivers.

In some specific but not limiting examples, the radar system employs only a single channel in the front end of the radar, and can implement a single-input-single-output (SISO) configuration, or a single-input-multiple-output (SIMO) configuration. In addition to performance amelioration, by doing so, processing times can be reduced and the need to deal with issues related to combining the two channels is circumvented.

In other embodiments, the present disclosure provides for optimizing localization of subjects as well as vital signs monitoring by using multiple-input-multiple-output (MIMO) configuration of the radar system, which may offer higher signal-to-noise ratios (SNR) and improved spatial resolution. The latter is achieved by employing orthogonal signals from multiple transmit antennas, using techniques such as time-division multiplexing (TDM) [27]. In a uniform linear array (ULA), by transmitting and receiving independent signals over a common signal path, this approach creates a larger virtual antenna array, whose effective size equals the product of the number of transmit and receive antennas, ultimately enhancing the radar's angular resolution.

Based on the developed approach, the technique of the present disclosure provides a complete methodology for human localization and accurate monitoring of their vital signs by leveraging prior knowledge of the FMCW signal structure, for the given problem.

Thus, according to one broad aspect of the of the present disclosure, it provides a monitoring system for use in monitoring vital signs of one or more subjects, the monitoring system comprising a control system configured for signal communication with a frequency modulated continuous wave (FMCW) radar to process measured data which is received from a single-channel front end of each of at least one receiver of said FMCW radar and which is in the form of data matrix indicative of consecutive beat signals, and provide output data indicative of vital signs of the subjects in a region of interest (ROI), said control system comprising a data processing utility comprising:
a localization module configured and operable to process the measured data indicative of said data matrix and provide support recovery data indicative of the received signals originated at localized one or more subjects; and
a vital signs monitoring module configured and operable to analyze the support recovery data and monitor vital signs of said localized one or more subjects.

In some embodiments, the localization module is configured and operable to utilize prior knowledge of typical subject's pulse and breathing frequencies to filter said measured data and extract a subject's data matrix relating to signals received by each of said at least one receiver of the radar from the subjects in a region of interest, and apply a joint-sparse recovery processing to data indicative of said subject's data matrix utilizing sparsity in the received signals, thereby providing said support recovery data indicative of the received signals originated at localized one or more subjects.

The extraction of the subject's data matrix typically includes determining Doppler information in the received signals returned from the subjects in the region of interest, said Doppler information being indicative of radial distance of each of said one or more subjects from the radar.

In some embodiments, the vital signs monitoring module may be configured and operable to apply to said support recovery data a frequency search for the vital signs based on cardiopulmonary activities. In some other embodiments, the vital signs monitoring module is configured and operable to apply a dictionary-based search for the vital signs over predetermined dictionary corresponding to frequency grids of the cardiopulmonary activities.

As noted above, the FMCW radar system may comprise a single channel and configured to localize multiple subjects at different distances from the FMCW radar; or may be configured to localize multiple subjects at different angular positions (azimuth angles) with respect to the radar.

The vital signs being monitored may for example include respiration rate (RR) and heartbeat rate (HR).

In some embodiments, the localization module is configured and operable to carry out the following:
pre-processing the measured data acquired during acquisition time interval Tᵢₙₜ and comprising the data matrix G×L of G chirps received from the region of interest in each of L acquisition frames, L defining a slow-time dimension of the data matrix G×L, said preprocessing comprising averaging values of G chirps, to thereby obtain a corresponding N×L data matrix Y in which N defines a fast-time dimension of the matrix Y;
processing the data matrix Y and detecting the one or more subjects in the region of interest and estimating spatial locations of said one or more subjects.

For example, the localization module is configured and operable to process the data matrix Y by carrying out the following:
utilizing said prior knowledge about the typical subject's pulse and breathing frequencies and performing spectral filtering of the data matrix Y along the slow-time dimension L of the data matrix Y, to thereby obtain the subject's data matrix \tilde{Y};
applying the joint-sparse recovery processing to the subject's data matrix \tilde{Y}, and obtaining a matrix \tilde{X} comprising complex amplitudes of the beat signals;
analyzing the matrix \tilde{X}, and determining the support recovery data S comprising a set of row coordinates m of matrix \tilde{X} associated with the one or more subjects in the region of interest; and
utilizing the matrix \tilde{X} and the support recovery data S and determining at least one of radial distance and azimuth angle with respect to the FMCW radar for each of M subjects, where m=1, . . . , M.

In some embodiments, the monitoring system comprises the frequency modulated continuous wave (FMCW) radar comprising at least one transmitter, each being configured to transmit series of millimeter wave signals to the region of interest in a clutter-rich environment, and one or more receivers associated with each of said at least one transmitter, each receiver being configured and operable to receive G chirps per acquisition frame returned from said region of interest within a field of view of the receiver, wherein the transceiver is operable to utilize, for each receiver, the single-channel front end thereof, and generate the measured data in the form of the data matrix indicative of the consecutive beat signals.

In some embodiments, said one or more subjects are moving subjects. In this case, said localization module may be configured and operable to carry out the following:
pre-processing the measured data, acquired during acquisition time interval Tᵢₙₜ, by dividing said acquisition time interval Tᵢₙₜ into L time windows, wherein each time window l, l=1, . . . , L, has constant velocity of subjects' movement and comprises G frames, thereby obtaining said data matrix having a size of N×G for each chirp received from the region of interest in each of said G frames, G defining a slow-time dimension of the data matrix N×G, and N defining a fast-time dimension of the data matrix, to thereby obtain a corresponding N×G data matrix Y₁, l=1, . . . , L;
processing the data matrix Y₁ and detecting the one or more moving subjects in the region of interest and estimating spatial locations of said one or more moving subjects.

For example, the localization module is configured and operable to process the data matrix Y₁ for all of said time windows l, l=1, . . . , L, by carrying out the following:
utilizing prior knowledge about typical subject's pulse and breathing frequencies and performing spectral filtering of the data matrix Y₁ along the slow-time dimension G of the data matrix Y₁, to thereby obtain a subject's data matrix \tilde{Y}ₗ;
applying joint-sparse recovery processing to the subject's data matrix \tilde{Y}ₗ, and obtaining a matrix \tilde{X}ₗ comprising complex amplitudes of the consecutive beat signals;
analyzing the matrix \tilde{X}ₗ, and determining the support recovery data S[l] comprising a set of row coordinates u of the 90matrix \tilde{X}ₗ associated with U subjects in the region of interest, where u=1, . . . , U; and
utilizing the matrix \tilde{X}ₗ and the support recovery data S[l] and determining at least one of radial distance and azimuth angle with respect to the FMCW radar for each of the U subjects.

In some embodiments, the FMCW radar is of a single-input-multiple-output (SIMO) or multiple-input-multiple-output (MIMO) configuration implementing a time-division multiplexing (TDM), thereby allowing implementation of a uniform linear array (ULA) setup. In these embodiments, said localization module may be configured and operable to carry out the following:

receive as input: Tᵢₙₜ, T_{loc}, T_{win}, {y[n, k, l]}, γ, L_{f}, Iₘₐₓ, B^{(R)}, B^{(H)} wherein Tᵢₙₜ is a predefined acquisition time interval of vital signs monitoring, T_{loc} is a duration of localization corresponding to an acquisition time of first L frames within said predefined acquisition time interval Tᵢₙₜ with a frame rate fₛ, T_{win} is a duration of a preceding time window corresponding to acquisition of L frames within the predefined acquisition time interval Tᵢₙₜ, {y[n, k, l]} represents measured data for n=1, . . . , N fast-time samples, k=1, . . . K receivers, and/=1, . . . , L slow-time frames, γ is a regularization parameter, L_{f} is a Lipschitz constant, Iₘₐₓ is a maximal number of iterations, B^{(R)} and B^{(H)} denote, respectively, frequency bands of respiration and heartbeat at rest;
at first T_{loc} perform the following:
   assemble matrices A and B and arrange said measured data {y[n, k, l]} to construct a 3D cube {Yₗ}=ₗ₌₁^{L=T}^{_{loc}}^{f}^{ₛ} to satisfy a model Yₗ=AXₗB+Wₗ, l=1, . . . , L, where A∈ [symbol] ^{N×M} is a known range-related Vandermonde matrix, M being a number of general radial distances, B∈ [symbol] ^{P×K} is a known angle-related matrix, Xₗ∈ [symbol] ^{M×P} is an unknown matrix of complex amplitudes where Xₗ(m, p)≙\tilde{x}_{m,p}[l], P being a number of general azimuth angles, and Wₗ∈ [symbol] ^{N×K} is a noise matrix;
   filter said 3D cube {Yₗ}=ₗ₌₁^{L=T}^{_{loc}}^{f}^{ₛ} utilizing said frequency bands of respiration and heartbeat at rest B^{(R)} and B^{(H)};
   utilizing Radar Localization of hUmans via Joint Sparse Recovery (RaLU-JSR) method to recover {Xₗ}ₗ₌₁^{L} and the support S, defined as a the set of 2D {m, p} indices whose cardinality corresponds to a certain number Z of individuals and whose indices point to range-angle locations {d^{(z)}, θ^{(z)}}_{z=1}^{Z} of respective individuals.

The vital signs monitoring module may be configured and operable to carry out the following for each predefined acquisition time interval Tᵢₙₜ after the preceding time window T_{win}: utilize the support S to evaluate the complex amplitudes {\hat{x}_{S(z)}[l]}_{z=1}^{\hat{Z}} corresponding to vital signs of each z'th subject, and the scaled approximations of the thoracic vibrations {\hat{v}_{z}}_{z=1}^{\hat{Z}} for each z'th detected subject; and estimate the vital pairs {f_{H}^{(z)}, f_{R}^{(z)}}_{z=1}^{Z} representing, respectively, the heart and respiration rates of each z'th subject at each time interval Tᵢₙₜ, given the thoracic vibrations {\hat{v}_{z}}_{z=1}^{\hat{Z}}, and the frequency bands of respiration and heartbeat at rest B^{(R)} and B^{(H)} using an extended VSDR (E-VSDR) method.

For example, said vital signs monitoring module is configured and operable to perform the following:
receive as input said scaled approximations of thoracic vibrations of Z detected subjects, {\hat{v}_{z}}_{z=1}^{\hat{Z}}, and B^{(R)}, B^{(H)};
for each z'th detected subject perform the following:
   for given frequency bands of respiration and heartbeat at rest B^{(R)} and B^{(H)}, express each extracted vibration \hat{v}_{z} as a linear combination of respiration and heartbeat dictionaries, D^{(R)} and D^{(H)}, respectively, each extracted vibration \hat{v}_{z} describing a frequency pattern of each z'th subject vibration;
   define the respiration support of the z'th subject, [symbol] _{R}^{(z)}, as;

$${𝒮}_{R}^{( z )} = {arg max}_{q = 1 , … , {Q}_{R}} \{ \vert {D}^{( R ) T} {\hat{v}}_{z} \vert \} ,$$

and define it as the respiration rate (RR) frequency estimate, \hat{f}_{R}^{(z)} by selecting the q'th frequency within a frequency subset defined by D^{(R)};
subtract the influence of respiration by defining a residual vector, \hat{v}′_{z}, as: \hat{v}′_{z}=\hat{v}_{z}− [symbol] , where d [symbol] _{R}^{(z)}∈ [symbol] ^{L} is the atom of D^{(R)} corresponding to [symbol] _{R}^{(z)} and [symbol] is the estimated amplitude over [symbol] _{R}^{(z)};
mitigate the impact of interfering respiratory harmonics on heartbeat rate (HR) by estimating the respective dictionaries D^{(R′,z)} and D^{(H′,z)}, being subsets of respectively, D^{(R)} and D^{(H)}, including interfering respiration harmonics and non-interfered heart frequencies, and subtracting their contributions to define the residual vector, \hat{v}″_{z}, including the heartbeat vibration as \hat{v}″_{z}=\hat{v}′_{z}−D^{(R′,z)} â_{z}^{(R′)}, where â_{z}^{(R′)} is the respective amplitude of D^{(R′,z)} describing each extracted vibration \hat{v}_{z};
estimate the heartbeat frequency of the z'th detected subject, \hat{f}_{H}^{(z)} defining it as the heartbeat support of the z'th subject defined as:

$${𝒮}_{H}^{( z )} = {arg max}_{q = 1 , … , {Q}_{{H}^{′} , z}} \{ \vert {D}^{( {H}^{′} , z ) T} {\hat{v}}_{z}^{″} \vert \} .$$

For example, said vital signs monitoring module is configured and operable to perform signal refinement procedure comprising the following:
for a monitoring time t, satisfying t>T_{ref}, where T_{ref} is a predetermined duration of monitoring, replace all vital estimates {\hat{f}_{H}^{(z)}, \hat{f}_{R}^{(z)}}_{z=1}^{Z} with the median value derived from the measured data collected up to the monitoring time t;
for each Tᵢₙₜ following T_{ref}:
   replace the vital estimates {\hat{f}_{H}^{(z)}, \hat{f}_{R}^{(z)}}_{z=1}^{Z} with the average of the estimations acquired in the last T_{avg}^{(H)} and T_{avg}^{(R)} seconds, respectively;
   replace the fixed bands of respiration and heartbeat, B^{(R)} and B^{(H)} respectively, with adaptive bands, B_{adp}^{(R)} and B_{adp}^{(H)} centered around the vital estimates {\hat{f}_{H}^{(z)}, \hat{f}_{R}^{(z)}}_{z=1}^{Z} with small frequency margins, the adaptive bands defined in [bpm] as: B_{adp}^{(R)}(\hat{f}_{R}^{(z)}≙[\hat{f}_{R}^{(z)}−ε_{R}\hat{f}_{R}^{(z)}+ε_{R}] and B_{adp}^{(H)}(\hat{f}_{H}^{(z)}≙[\hat{f}_{H}^{(z)}−ε_{H}\hat{f}_{H}^{(z)}+ε_{H}], respectively, where ε_{R} and ε_{H} are predefined scalars which determine the margins of the corresponding bands.

According to another broad aspect of the invention, it provides a monitoring system for non-contact vital signs monitoring of one or more subjects comprising:
a frequency modulated continuous wave (FMCW) radar comprising at least one transmitter, each being configured to transmit series of millimeter wave signals to a region of interest in a clutter-rich environment, and one or more receivers associated with each of said at least one transmitter, each receiver being configured and operable to receive signals returned from said region of interest within a field of view of the receiver, wherein the transceiver is operable to utilize, for each receiver, a single-channel front end thereof, and generate measured data in the form of data matrix indicative of consecutive beat signals; and
a control system configured and operable to receive and process said measured data and provide output data indicative of vital signs of the subjects in the region of interest, wherein the control system comprising a data processing utility comprising: a localization module configured and operable to process input data indicative of said data matrix and determine recovery data indicative of the received signals originated at localized one or more subjects; and a vital signs monitoring module configured and operable to monitor vital signs of said localized one or more subjects;
the system being characterized by at least one of the following:
the localization module is configured and operable to utilize prior knowledge of typical subject's pulse and breathing frequencies to filter said input data and extract a subject's data matrix relating to the received signals returned from the subjects in the region of interest, and applying a joint-sparse recovery processing to said subject's data matrix utilizing sparsity in the returned signals, thereby providing said support recovery data indicative of the received signals originated at the localized one or more subjects; and
the vital signs monitoring module is configured and operable to analyze the support recovery data by carrying out one of the following: applying to said support recovery data a frequency search for the vital signs based on cardiopulmonary activities; and applying a dictionary-based search for the vital signs over predetermined dictionary corresponding to frequency grids of the cardiopulmonary activities.

Thus, the present disclosure, in some of its aspects, provides the localization technique which utilizes a frequency-based understanding of human cardiopulmonary activity, as well as the sparse nature of the data via a joint sparse recovery (JSR) mechanism [18]. This approach allows for computationally efficient extraction of the relevant Doppler samples throughout the complete monitoring process.

Then, by conducting for example the appropriate frequency search, the inventors exhibit high-resolution NCVSM of multiple people, given their extracted thoracic vibrations. The frequency search may utilize a Vital Signs based Dictionary Recovery (VSDR) approach developed by the inventors.

The performance of the proposed methodology is verified through simulations that incorporate synthetic signals based on the developed model with in vivo data of 30 monitored individuals from reference [19]. The present disclosure demonstrates both precise human localization in a multiple object scenario and superior accuracy results for RR and HR monitoring, when compared to state-of-the-art techniques using several statistical metrics.

Throughout the disclosure, the following notation is used: Scalars are denoted by lowercase letters (a), vectors—by boldface lowercase letters (a), sets are given by calligraphic font(S), and matrices are denoted by boldface capital letters (A). The (i,j)'th element of a matrix A is written as A(i, j), and aₗ is the l'th column of A. The notations (⋅)^{T}, and (⋅)^{H} indicate the transpose and Hermitian operations, respectively.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting examples only, with reference to the accompanying drawings, in which:

FIG. 1A shows schematically, by way of a block diagram, an exemplary monitoring system of non-contact vital signs monitoring (NCVSM) of multiple people in a cluttered environment according to the present disclosure;

FIG. 1B more specifically exemplifies, by way of a block diagram, functional parts of the monitoring system of the present disclosure;

FIG. 2 exemplifies a flow diagram of the method of the present disclosure using FMCW radar in a SISO (Single Input Single Output) configuration to localize multiple subjects with respect to the radar;

FIG. 3 shows a flow diagram of the method of the present disclosure using FMCW radar in a SIMO (Single Input Multiple Output) configuration to localize multiple subjects at different azimuthal angles with respect to the radar;

FIGS. 4A and 4B schematically exemplify the main components of FMCW radar and the examined multiple objects scenario, respectively;

FIG. 4C exemplifies the transmitted and received chirp sequences at each frame, wherein: the brackets illustrate the case of transmitting G consecutive chirps per frame, instead of a single one; the top graph shows frequency sawtooth waveform; the middle graph shows analog beat signal after mixing; and the bottom graph shows discrete beat signal after ADC sampling;

FIG. 5 exemplifies, by way of a block diagram, the NCVSM system for monitoring multiple individuals via the use of FMCW radar, wherein: right side of the figure, blocks 1.a to 1.e, describes the method of the present disclosure yielding accurate estimates of respiration rate (RR) and heart rate (HR) by exploiting the sparse composition of the input data in accordance with human cardiopulmonary properties and using only a single channel; left side of the figure, blocks 2.a to 2.g, describes the conventional framework which lacks adequate theoretical explanations and presents difficulties in dealing with noisy, cluttered environments;

FIGS. 6A to 6B show, respectively, real impedance and ECG data [19], wherein FIG. 6A shows impedance [Ω] used as a reference for comparing the RR estimates and to simulate human thoracic vibration; and FIG. 6B shows ECG [mV] used as a reference for comparing the HR estimates;

FIGS. 7A to 7B exemplify multiple people localization by the setup of Table 1, for SNR=0 [dB], wherein: FIG. 7A shows the Range vs. Slow-Time map via the magnitudes of \hat{X}_{M}, the row-wise intensities corresponding to the DC component and the reflections of Table I's objects; and FIG. 7B compares several known localization techniques with the technique of the present disclosure;

FIGS. 8A to 8E exemplify the performance of NCVSM system based on subject GDN0004's data for SNR=1 [dB], compared to the references, using VSDR technique of the present disclosure and other state-of-the-art techniques, wherein: FIG. 8A shows the extracted thoracic vibration v₂ of human #2 from Table 1; FIG. 8B shows results of FFT-based peak selection with zero-padding; FIG. 8C shows results of FFT-based peak selection without zero-padding; FIG. 8D shows results by the method detailed in reference [8] called here Phase-Reg; and FIG. 8E shows results by the VSDR (Vital Signs based Dictionary Recovery) method of the present disclosure;

FIGS. 9A to 9H illustrate comparison between HR and RR monitoring performance using the techniques of FIGS. 8B to 8E as a function of SNR (SNR∈[−2,2]) according to several statistical metrics, wherein: FIG. 9A shows HR Success Rate; FIG. 9B shows HR PCC (Pearson Correlation Coefficient); FIG. 9C shows HR MAE (Mean-Absolute Error); FIG. 9D shows HR RMSE (Root-Mean-Square Error); FIG. 9E shows RR Success Rate; FIG. 9F shows RR PCC; FIG. 9G shows RR MAE; and FIG. 9H shows RR RMSE;

FIG. 10 exemplifies the process of multiple people localization by the setup of Table 5, for SNR=0 [dB] using the JSR (Joint Sparse Recovery) technique of the present disclosure compared to known-in-the-art approaches: intensity-based Angle-FFT method [7] and MUSIC-DOA approach [22-23];

FIGS. 11A to 11O exemplify the performance of NCVSM system compared to the references (ground truth) by VSDR and the other examined techniques, for SNR=0 [dB]. Data are shown for humans listed in Table 5, human #1 (FIGS. 11A to 11E), human #2 (FIGS. 11F to 11J), and human #3 (FIGS. 11K to 11O) respectively; wherein

FIGS. 11A, 11F, and 11K show the respective vectors v of thoracic vibration patterns of each detected human; for each detected human, respectively: FIGS. 11B, 11G, and 11L show estimated vital signs obtained by the VSDR approach; FIGS. 11C, 11H, and 11M show estimated vital signs obtained by the FFT-based peak selection with zero-padding (ZP) approach; FIGS. 11D, 11I, and 11N show estimated vital signs obtained by the FFT-based peak selection without ZP approach; and FIGS. 11E, 11J, and 11O show estimated vital signs obtained by Phase-Reg approach detailed in [8], based on the phase of an FMCW signal;

FIGS. 12A to 12H illustrate comparison between HR and RR monitoring performance using the techniques of FIGS. 11A to 11O as a function of SNR (SNR∈[−2,2]) according to several statistical metrics, wherein: FIG. 12A shows HR Success Rate; FIG. 12B shows HR PCC (Pearson Correlation Coefficient); FIG. 12C shows HR MAE (Mean-Absolute Error); FIG. 12D shows HR RMSE (Root-Mean-Square Error); FIG. 12E shows RR Success Rate; FIG. 12F shows RR PCC; FIG. 12G shows RR MAE and

FIG. 12H shows RR RMSE;

FIG. 13 is a photo of an experimental setup, where two vibrating plates, imitating two humans, are placed at radial distance 0.95 [m] from the radar and at azimuth angles +−13 [deg], each plate vibrating according to a different real human breathing pattern;

FIGS. 14A to 14F exemplify the process of multiple people localization by the setup of FIG. 13 using the JSR (Joint Sparse Recovery) technique of the present disclosure compared to known state of-the-art approaches: MUSIC-DOA approach [22-23] and intensity-based Angle-FFT method [7], wherein FIGS. 14A, 14C, 14E show the localization maps (Range vs. Angle) with JSR, MUSIC-DOA and Angle-FFT methods, respectively; and FIGS. 14B, 14D, 14F show the respective localization curves (Magnitude at localized “person” vs. Azimuthal angle) obtained from the maps of FIGS. 14A, 14C, 14E;

FIGS. 15A to 15N exemplify the performance of NCVSM system compared to the references (ground truth) by VSDR and the other examined techniques wherein data are shown for “humans” as per the experimental setup of FIG. 13, for human #1 (FIGS. 15A to 15G) and human #2 (FIGS. 15H to 15N), wherein FIGS. 15A and 15H show the respective vectors v₁ and v₂ of thoracic vibration patterns of each detected human; and wherein for each detected human, respectively: FIGS. 15B and 15I show estimated vital signs obtained by the VSDR approach; FIGS. 15C and 15J show estimated vital signs obtained by Phase-Reg approach detailed in [8], based on the phase of an FMCW signal; FIGS. 15D and 15K show estimated vital signs obtained by the FFT-based peak selection without ZP approach; and FIGS. 15E and 15L show estimated vital signs by the FFT-based peak selection with zero-padding (ZP) approach;

FIGS. 15F, 15G, 15M and 15N illustrate comparison between HR and RR accuracy using the techniques of FIGS. 15B to 15E and FIGS. 15I to 15L according to several statistical metrics, wherein FIGS. 15F and 15M show HR MAE (Mean-Absolute Error) and RMSE (Root-Mean-Square Error), and FIGS. 15G and 15N show RR MAE (Mean-Absolute Error) and RMSE (Root-Mean-Square Error);

FIG. 16 shows FMCW frame sequences, each window contains G frames, the duration of a chirp, frame, and window is T_{c}, T_{g} and Tₛ, respectively; each chirp is sampled to a length N discrete signal with sampling interval T_{f};

FIGS. 17A and 17B show a simulation scenario wherein FIG. 17A shows a cluster composed of 20 scatterers used for simulating echoes from a chest; and FIG. 17B shows the range-FFT map of two moving persons;

FIGS. 18A and 18B show localized bins by selecting peaks from range-FFT map (FIG. 18A) and using joint sparsity recovery (Eq. (62)) (FIG. 18B);

FIG. 19 shows estimated distance and velocity from phases based on range-FFT and joint sparsity, wherein the first and second row show estimated distance and velocity of subject 1, and the third and fourth row show estimated distance and velocity of subject 2;

FIG. 20 shows heartbeat signal extraction of subject 1 and subject 2 with different methods;

FIG. 21 is a schematic illustration of the main components of a MIMO ULA FMCW radar and a multi-object scenario, with objects located at varying angles and radial distances from the radar;

FIG. 22 is an illustration of the outcome of a time division multiplexing (TDM) technique, which creates a virtual 1×8 SIMO ULA given a physical 2×4 MIMO ULA;

FIGS. 23A and 23B show by way of a block diagram the proposed algorithm for robust multi-person localization and vital signs monitoring using SIMO or MIMO ULA FMCW Radar;

FIG. 24 is an illustration of the spectral components of respiration and heartbeat frequency bands B^{(R)} and B^{(H)}, respectively, specifically it shows possible overlap between heartbeat components and high-order respiratory harmonics;

FIG. 25 shows an example of an impedance signal from that was used to generate realistic mechanical displacements for the vibration unit corresponding to changes in human thoracic volume;

FIG. 26A is a block diagram of the developed phantom of the present disclosure showing the key components of a single vibration unit; and FIG. 26B shows an overview of the validation process, comprising a radar, up to three vibration units, and a GUI for control;

FIG. 27 shows experimental setups in a cluttered demonstration room, wherein (c1) single-person phantom trials, (c2) Single-person human trials, (c3) multi-person phantom trials, and (c4) multi-person human trials;

FIGS. 28A to 28F show localization maps of multi-person phantom trial of class c3, trial #2; The maps were produced by Angle-FFT (FIG. 28A), MUSIC (FIG. 28B), SOD-MUSIC (FIG. 28C), LCMV (FIG. 28D), cal-CIR (FIG. 28E) and the proposed RaLU-JSR (Algorithm 1) (FIG. 28F); The X and 0 signs denote the estimated and true locations of humans, respectively;

FIGS. 29A to 29H, 30A to 30H, and 31A to 31H, show NCVSM of 3 subjects, respectively, for multi-person phantom trial-class c3, trial #2; Extracted thoracic vibrations v1-v3 are shown for some Tᵢₙₜ, PhaseReg (FIGS. 29B, 30B, and 31B), FFT (FIGS. 29C, 30C, and 31C), OrthProj (FIGS. 29D, 30D, and 31D), the refined versions PhaseReg+ (FIGS. 29E, 30E, and 31E), FFT+ (FIGS. 29F, 30F, and 31F), and OrthProj+ (FIGS. 29G, 30G, and 31G), and the proposed E-VSDR estimates (FIGS. 29H, 30H, and 31H);

FIGS. 32A to 32D show NCVSM performance plots for multi-person phantom trials c3, wherein FIGS. 32A and 32B show the average empirical CDFs for HR

(FIG. 32A) and RR (FIG. 32B) estimations; and FIGS. 32C and 32D show RMSE scores: subject-wise with class average and median, for both HR (FIG. 32C) and RR (FIG. 32D) estimations;

FIGS. 33A to 33F show localization maps of multi-person phantom trial of class c4, trial #3; The maps were produced by Angle-FFT (FIG. 33A), MUSIC (FIG. 33B), SOD-MUSIC (FIG. 33C), LCMV (FIG. 33D), cal-CIR (FIG. 33E) and the proposed RaLU-JSR (Algorithm 1) (FIG. 33F); The X and 0 signs denote the estimated and true locations of humans, respectively;

FIGS. 34A to 34H, 35A to 35H, and 36A to 36H, show NCVSM of 3 subjects, respectively, for multi-person phantom trial-class c4, trial #3; Extracted thoracic vibrations v1-v3 are shown for some Tᵢₙₜ, PhaseReg (FIGS. 34B, 35B, and 36B), FFT (FIGS. 34C, 35C, and 36C), OrthProj (FIGS. 34D, 35D, and 36D), the refined versions PhaseReg+ (FIGS. 34E, 35E, and 36E), FFT+ (FIGS. 34F, 35F, and 36F), and OrthProj+ (FIGS. 34G, 35G, and 36G), and the proposed E-VSDR estimates (FIGS. 34H, 35H, and 36H);

FIGS. 37A to 37D show NCVSM performance plots for multi-person phantom trials c4, wherein FIGS. 37A and 37B show the average empirical CDFs for HR

(FIG. 37A) and RR (FIG. 37B) estimations; and FIGS. 37C and 37D show RMSE scores: subject-wise with class average and median, for both HR (FIG. 37C) and RR (FIG. 37D) estimations;

FIGS. 38A to 38F show localization maps of single-person phantom trial of class c1, trial #2; The maps were produced by Angle-FFT (FIG. 38A), MUSIC (FIG. 38B), SOD-MUSIC (FIG. 38C), LCMV (FIG. 38D), cal-CIR (FIG. 38E) and the proposed RaLU-JSR (Algorithm 1) (FIG. 38F); The X and 0 signs denote the estimated and true locations of humans, respectively;

FIGS. 39A to 39H show NCVSM for a single-person phantom trial—class c1, trial #2; Extracted thoracic vibration \hat{v}₁ is shown for some Tᵢₙₜ, PhaseReg (FIG. 39B), FFT (FIG. 39C), OrthProj (FIG. 39D), the refined versions PhaseReg+ (FIG. 39E), FFT+ (FIG. 39F), and OrthProj+ (FIG. 39G), and the proposed E-VSDR estimates (FIG. 39H);

FIGS. 40A to 40D show NCVSM performance plots for a single person phantom trials c3, wherein FIGS. 40A and 40B show the average empirical CDFs for HR

(FIG. 40A) and RR (FIG. 40B) estimations; and FIGS. 40C and 40D show RMSE scores: subject-wise with class average and median, for both HR (FIG. 40C) and RR (FIG. 40D) estimations;

FIGS. 41A to 41F show localization maps of single-person phantom trial of class c1, trial #7; The maps were produced by Angle-FFT (FIG. 41A), MUSIC (FIG. 41B), SOD-MUSIC (FIG. 41C), LCMV (FIG. 41D), cal-CIR (FIG. 41E) and the proposed RaLU-JSR (Algorithm 1) (FIG. 41F); The X and 0 signs denote the estimated and true locations of humans, respectively;

FIGS. 42A to 42H show NCVSM for a single-person phantom trial-class c1, trial #7; Extracted thoracic vibration \hat{v}₁ is shown for some Tᵢₙₜ, PhaseReg (FIG. 42B), FFT (FIG. 42C), OrthProj (FIG. 42D), the refined versions PhaseReg+ (FIG. 42E), FFT+ (FIG. 42F), and OrthProj+ (FIG. 42G), and the proposed E-VSDR estimates (FIG. 42H); and

FIGS. 43A to 43D show NCVSM performance plots for a single person phantom trials c2, wherein FIGS. 43A and 43B show the average empirical CDFs for HR

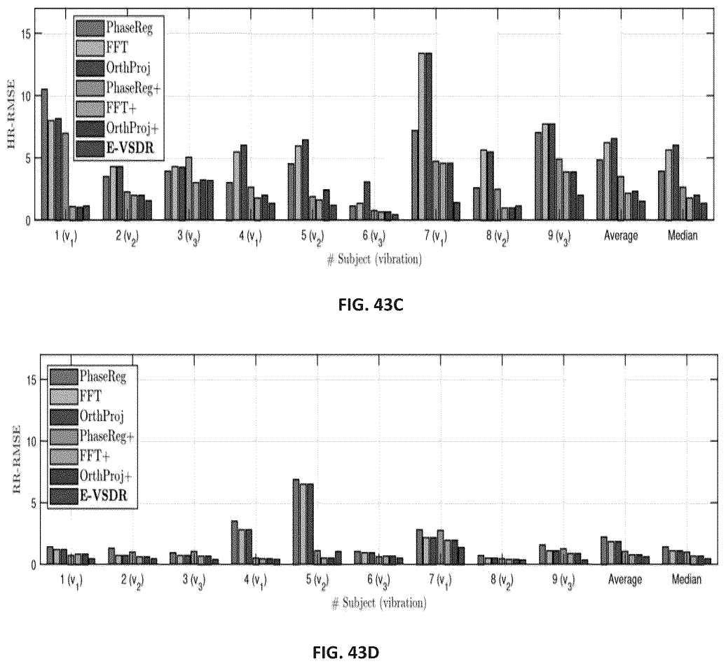

(FIG. 43A) and RR (FIG. 43B) estimations; and FIGS. 43C and 43D show RMSE scores: subject-wise with class average and median, for both HR (FIG. 43C) and RR (FIG. 43D) estimations.

### DETAILED DESCRIPTION OF EMBODIMENTS

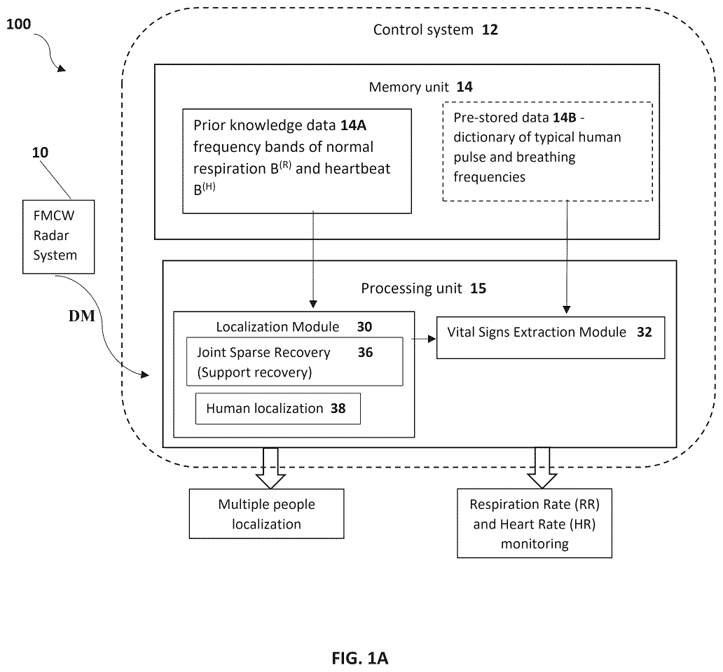

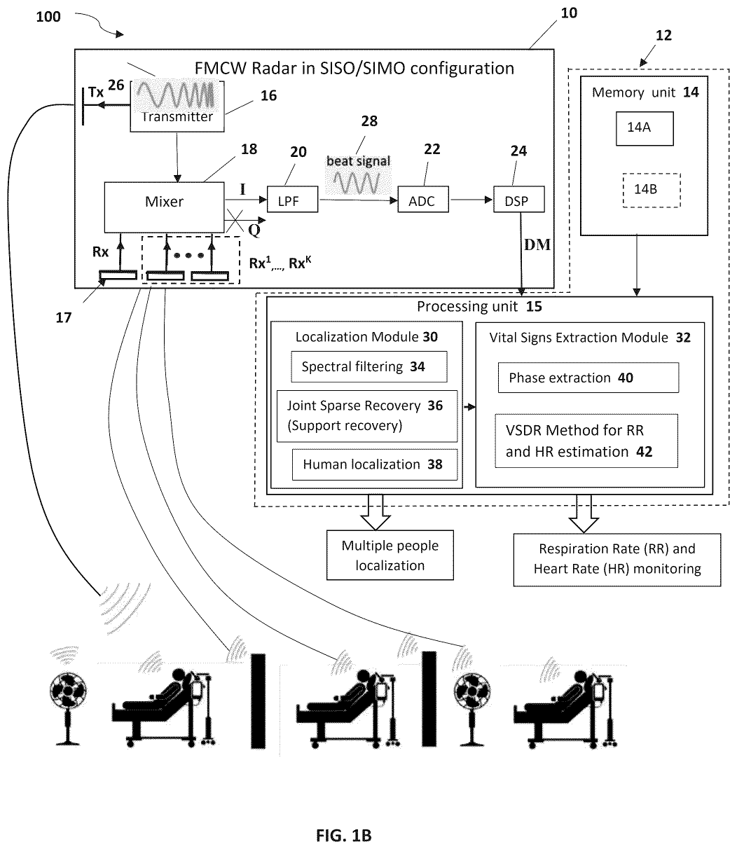

Reference is made to FIG. 1A showing schematically, by way of a block diagram, the main functional components of a non-contact vital signs monitoring (NCVSM) system 100 configured and operable according to the technique of the present disclosure. The monitoring system 100 includes a frequency modulated continuous wave (FMCW) radar system 10, and a control system 12.

As will be described more specifically further below with reference to FIG. 1B, the FMCW radar 10 includes at least one signal transmitter producing a transmitting signal in which the frequency varies linearly with time for irradiating one or more subjects (individuals) being monitored in a region of interest, and one or multiple signal receivers receiving signal responses from the region of interest. The radar 10 performs some signal processing to generate output data in the form of acquired data matrix DM. This data is received and processed by the control system 12.

The control system 12 is typically a computerized system which is in signal/data communication (via wires or wireless communication of any known suitable type) with the radar system 10. The control system includes inter alia data input and output utilities (not shown), memory 14, and a processing utility 15.

The processor utility 15 is configured and operable according to the present disclosure to receive and process the acquired data matrix DM and determine various subject's parameters and conditions, in particular respiration rate (RR) and heart rate (HR) of each individual being monitored. The processor unit 15, configured according to the technique of the present disclosure, includes a localization module 30 and a non-contact vital signs monitoring/extraction module 32, and provides simultaneous vital signs monitoring of several subjects at different radial distances from the radar system 10 and/or at the same distance but at different angular positions (azimuthal angles), while in a realistic environment containing clutter and noise.

More specifically, the localization module 30 is configured and operable to localize each subject in the region of interest, using a joint-sparse recovery (JSR) mechanism/utility 36 and a subject's (e.g., human) localization mechanism 38. As shown in the figure, and will be described more specifically further below, the subject's localization mechanism 38 utilizes prior knowledge data 14A of typical human pulse and breathing frequencies (i.e., frequency bands of normal respiration B^{(R)} and heartbeat B^{(H)}) which data is stored in the memory 14. Here, B^{(R)}, B^{(H)}∈[0 fₛ/2) are the typical pulse and breathing frequency ranges, aiding in the separation of humans from static or vibrating clutter, such as fans (fₛ being slow-time frame rate, as described below).

The non-contact vital signs monitoring module 32 is configured and operable to monitor vital signs of each subject utilizing predetermined data, stored in the memory 14. To this end, in some examples, dictionary 14B of typical human pulse and breathing frequencies can be used (also pre-stored in the memory), as will also be described more specifically further below.

Reference is made to FIG. 1B, illustrating more specifically an exemplary NCVSM system 100 according to the technique of the present disclosure. To facilitate understanding, the same reference numbers are used to identify the components which are identical (functionally identical) in all the examples described herein.

The NCVSM system 100 exemplified in FIG. 1B is configured generally similar to the system of FIG. 1A, namely includes an FMCW radar system 10 and a control system 12. In this specific not-limiting example, the FMCW radar 10 includes a single signal transmitter 16 producing a transmitting signal Tx in which the frequency is varied linearly with time. For example, the frequency of the transmitted signal may increase at a constant linear ramp rate from 77 GHz to 81 GHz in a period of about 50 microseconds. This transmit signal Tx is referred to as a ramp signal or a chirp signal 26.

In a typical operation scenario, the signal 26 transmitted by the FMCW transmitter 16 inside a cluttered environment of the region of interest may be scattered (and/or reflected) by one or more obstacles (e.g., humans, walls, vibrating fans, etc.). The total response signal (scattered/reflected signal) Rx is received by one or more receive units 17 (multiple such units being shown in the figure) in the FMCW radar system 10.

A baseband signal is obtained from a mixer 18 which mixes the transmitted signal 26 with the received scattered signal Rx to create what is termed an intermediate frequency (IF) signal or a beat signal 28. The beat signal 28 is used to determine the acquired data matrix DM to be processed by the control system 12. In general, the frequency of the beat signal 28 is proportional to the range (distance) of the obstacle(s).

More specifically, the beat signal 28 is a signal formed by a conditioning circuit, which includes an anti-alias (low pass) filter 20 and an amplifier (not shown), and is then sampled by an analog to digital converter (ADC) 22 and processed by a digital signal processor (DSP) 24 to assemble the acquired data matrix DM for further processing by the control system 12 (its processing unit 15).

Traditionally, FMCW radar receivers use both an in-phase (I) and a quadrature-phase (Q) channels in their receivers. Using both channels allow forming I and Q components of the received signals to generate a beat signal which includes both phase and amplitude data without a loss of information. However, I/Q imbalances are known to occur due to mismatches between the parallel sections (or channels) of the receiver chain providing the I and the Q signal paths. The lack of perfect orthogonality and difference in gain levels in each channel may corrupt the desired information to be extracted.

Therefore, the technique of the present disclosure utilizes only a single channel of the receiver, e.g., the In-phase (I) beat signal 28 thus reducing processing times and avoiding the complications related to combining the two channels.

The processing unit 12 configured according to the technique of the present disclosure includes the localization module 30 and vital signs extraction module 32 that provide simultaneous vital signs monitoring of several people (generally subjects) located either at different radial distances from the radar or at the same distance but at different azimuthal angles, in a realistic environment containing clutter and noise.

The localization utility/module 12 includes a joint-sparse recovery (JSR) mechanism 36 (support recovery), and a subject's localization mechanism 38, and preferably also includes a spectral filtering module 34. The latter is configured to use prior knowledge of typical subject (human) pulse and breathing frequencies 14A to perform spectral filtering of the input data matrix DM in the slow-axis, defined by the time period T_{c} of a single chirp 26 and representing the temporal variation of the human thorax between successive chirps, as will be described in more detail below.

Since body surface displacement due to vital signs is of small amplitudes
(<10 mm) and low frequency (<4 Hz), it is assumed that there would be no appreciable change in phase of human thorax vibration during the chirp time (defining the fast-time axis of the matrix DM, along the row coordinates) and measuring phase changes induced between successive chirps (slow-time axis, along the column coordinates of DM) would be sufficient. Also, this step uses the prior knowledge of typical human pulse and breathing frequencies to aid in the separation of humans from static or vibrating clutter, such as fans.

The Joint Sparse Recovery (JSR) mechanism 36 has been developed by the inventors to recover the slow-time varying complex amplitudes of human thoracic vibrations from the full data set obtained from the radar system by utilizing the sparse properties of the input signal, within the frequency limits that characterize normal respiration and heartbeat. The technique of the present disclosure provides a model which assumes that the humans being monitored are stationary except for minor movements caused by breathing or speaking. The latter induces a row-wise sparsity in the data matrix DM indicative of the received signals from the region of interest, and allows using constant row coordinates (called “support” in the following) of the data matrix associated with humans in the radar's field of view. The technique of support recovery will be detailed further below.

The human localization mechanism 38 performs calculation of an efficient recovery of only the human-related Doppler samples from the data matrix DM (using the constant row-coordinates or “support” obtained by JSR 36) to be used throughout the remainder of the monitoring process. This processing step provides more efficient data handling compared to known-in-the-art techniques which perform the localization based on the entire data provided by the radar system, containing the full map of frequencies corresponding to object distances (fast-time along the row-indexes) and thoracic vibrations (slow-time along the column indexes).

According to the present disclosure, the efficiency is achieved thanks to the recognition by the inventors of the row-wise sparsity of the data matrix, i.e., the property of the model satisfying the condition K<<M, where K is the number of objects in the radar's field of view and M is the maximal number of resolvable distances by the radar defined by the chirp's total bandwidth.

The Vital Signs Extraction Module 32 is configured to continuously evaluate the vital signs, e.g., the respiratory rate (RR) and the heartbeat rate (HR). Since the human Doppler information is modulated in the phase of a complex exponential wave, once the human-related Doppler samples are extracted from the data matrix DM, a phase extraction module 40 is used configured and operable to estimate the phases of the corresponding human by known in the art methods.

To this end, Vital Signs Extraction Module 32 may utilize vital signs-based dictionary recovery (VSDR) mechanism 42 to effectively utilize pre-stored dictionary 14B for the vital signs over high-resolution frequency grids corresponding to resting cardiopulmonary activity, or other frequency estimation mechanisms, for example those including MUSIC and annihilating filter.

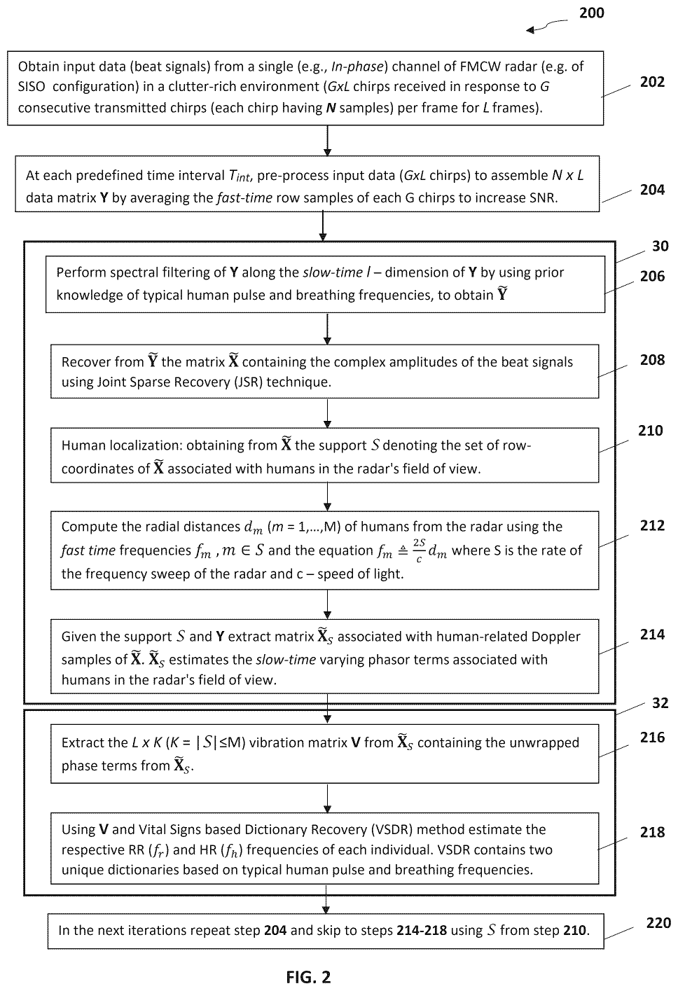

Reference is made to FIG. 2 showing a flow diagram 200 of an exemplary method of the present disclosure where the FMCW radar (10 in FIGS. 1A and 1B) includes a single transmitter and a single receiver, and is configured and operable to implement a SISO configuration. It is noted that this method allows for only a single object detection at any radial distance from the radar.

The method starts with obtaining input data (beat signals) from a single (e.g., In-phase) channel of the SISO FMCW radar in a clutter-rich environment. This input data (measured data) is indicative of G×L chirps, received by the radar's receiver in response to G consecutive transmitted chirps (each chirp having N samples) per frame for L frames to obtain (step 202). At each predefined time interval, Tᵢₙₜ, the input data is pre-processed to assemble N×L data matrix Y by averaging the fast-time row samples of each of G chirps to increase SNR (step 204).

The next steps, 206 to 214, of the signal processing are directed at detecting the humans in the clutter-rich environment and estimating their spatial location. In step 206, spectral filtering of data matrix Y is performed along the slow-time l-dimension of data matrix Y, by using prior knowledge of typical human pulse and breathing frequencies, to thereby obtain a matrix \tilde{Y}. In step 208, matrix \tilde{X} containing the complex amplitudes of the beat signals is recovered from the matrix \tilde{Y} using the Joint Sparse Recovery (JSR) technique of the present disclosure which will be described in detail below. Human localization is performed in step 210 by obtaining from the matrix \tilde{X}, the support S denoting the set of row-coordinates of matrix \tilde{X} associated with humans in the radar's field of view. Radial distances dₘ (m 1, . . . , M) of humans from the radar are determined in step 212 using the fast time frequencies fₘ, m∈S and the equation:

$${f}_{m} {=}^{Δ} \frac{2 S}{c} {d}_{m}$$

where S (not to be confused with the support S) is the rate of the frequency sweep of the radar and c is the speed of light.

Given the support S and matrix Y, a matrix \tilde{X}_{S} is extracted (step 214) being associated with human-related Doppler samples of matrix \tilde{X}. The matrix \tilde{X}_{S} estimates the slow-time varying phasor terms associated with humans in the radar's field of view.

The last steps 216 and 218 of the signal processing are directed at determining/monitoring the vital signs (here, RR and HR) of the previously located humans. In step 216, the L×K (K=|S|≤M) vibration matrix V is extracted from the matrix \tilde{X}_{S} containing the unwrapped phase terms. In the last step 218, the novel Vital Signs based Dictionary Recovery (VSDR) technique and the vibration matrix V are used to estimate the respective vital signs, RR (fᵣ) and HR (fₕ) frequencies, of each individual, as will be described in detail further below.

VSDR contains two unique dictionaries based on typical human pulse and breathing frequencies. In the next iterations of the non-contact monitoring procedure (step 220), step 204 of input data pre-processing is repeated and one can skip to steps 214 to 218 using the support S from step 210. Since in the present disclosure stationary subjects are monitored, thus the coordinates of the support S are fixed throughout the monitoring procedure.

Reference is made to FIG. 3 showing a flow diagram 300 of an exemplary method of the present disclosure where the FMCW radar (10 in FIGS. 1A and 1B) includes a single transmitter and multiple receivers, and is configured and operable to implement a SIMO configuration. This method allows, in addition to estimating the radial distance of the subjects from the radar, to estimate their azimuthal angle as well, while monitoring their vital signs.

The method starts (step 302) with obtaining input data (beat signals) from a single (e.g., In-phase) channel of SIMO FMCW radar in a clutter-rich environment using K>1 receivers by sending L signal frames in total (with one chirp per frame). At each predefined time interval, Tᵢₙₜ, the input data is pre-processed to assemble KM×L data matrix Y with an increased SNR (step 204). This can be based on averaging of G consecutive chirps for each frame, as described below. Steps 306 to 310 are similar to the respective steps 206 to 210 of the above-described method 200, and in step 312 the localization of subjects involves determination of the radial distance and the azimuth angle of each monitored individual with respect to the radar. Steps 314 to 320 are similar to the respective steps 214 to 220 of the above-described method 200.

In the following, the standard 2-D FMCW model for single human RR and HR monitoring at a given time window is presented, based on previous works. Then, existing processing methods that employ this model are reviewed. Then, the technique of the present disclosure including an extended representation for multiple people and clutter and the proposed NCVSM method is described in detail.

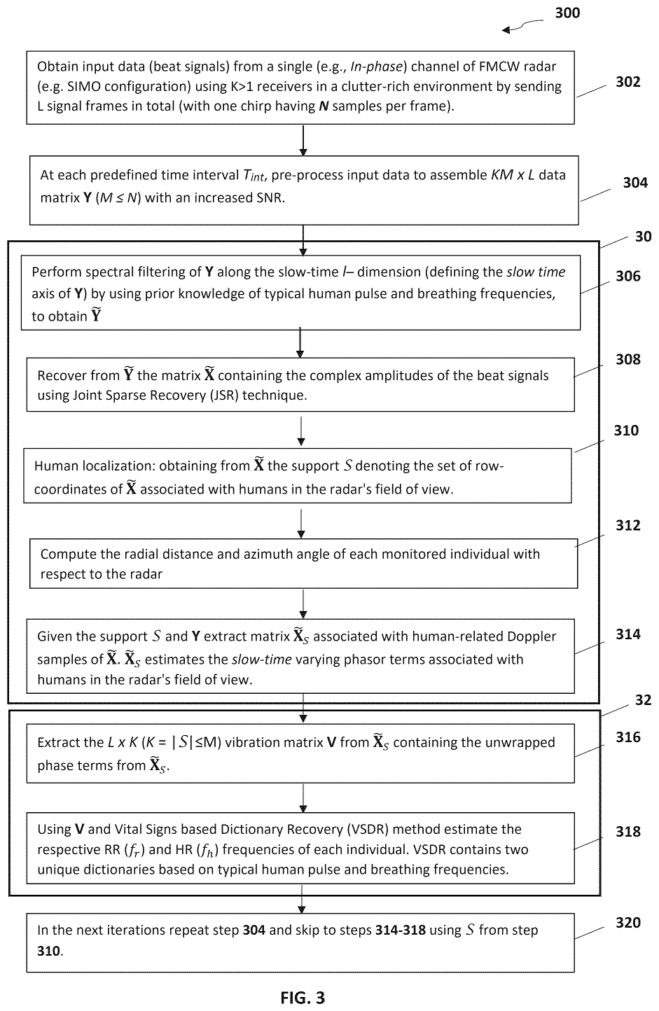

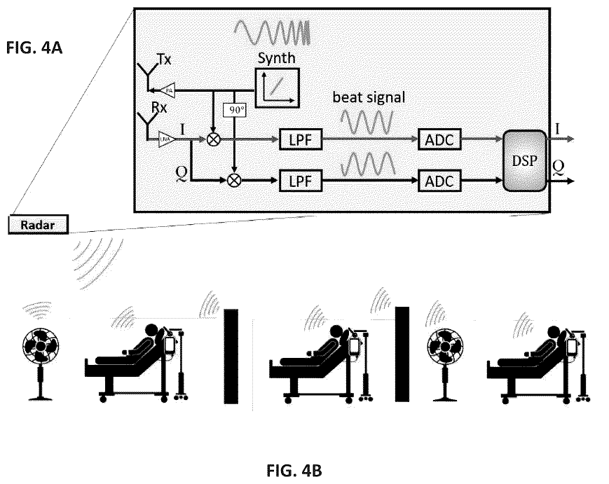

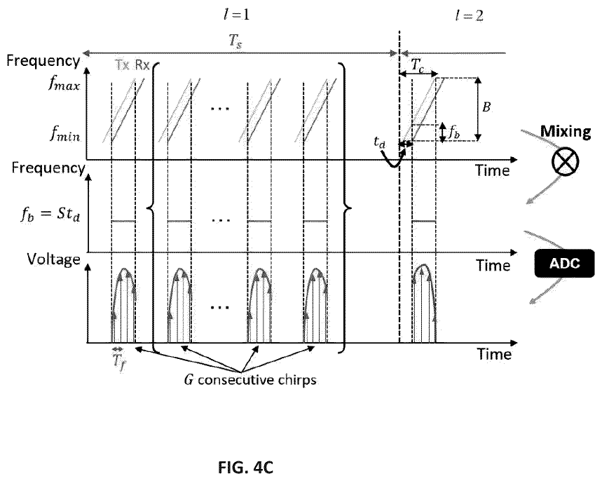

Reference is made to FIGS. 4A and 4B showing a typical FMCW radar transmitting a series of signals at a given time frame, called chirps, whose instantaneous frequency increases linearly over time, forming a saw-tooth waveform. The reflected echo signals split between the I/Q channels through which they are mixed with versions of the transmitted one followed by a lowpass-filter (LPF) to obtain analog base-band signals, known as the intermediate-frequency (IF) signals [13], [20], which are also called beat signals [11], [15], as will be used in this disclosure. The beat signals in each frame are sequentially sampled by the ADC, resulting in discrete base-band signals corresponding to each channel, which are sent from the radar to a local computer for further processing.

Considering a static human target located at distance d₀ (e.g., do meters) from the radar, with the antenna facing its thorax, the phenomena of respiration and heartbeat produce small time-varying changes in its relative position (with respect to the radar), so that its actual (effective) radial distance from the radar is:

$$d ( t ) = {d}_{0} + v ( t ) , \qquad ( 1 )$$

where v(t), being v(t)<<d₀, denotes a human thoracic vibration due to its cardiopulmonary activity. After hitting a human thorax, a transmitted chirp is reflected back to the radar's receiver and the received signal appears as an attenuated and shifted version of the transmitted chirp.

FIG. 4C exemplifies the transmitted and received chirp sequences at each frame. Here: a part of the figure defined by brackets illustrates the case of transmitting G consecutive chirps per frame, instead of a single one; the top graph shows frequency sawtooth waveform; the middle graph shows analog beat signal after mixing; and the bottom graph shows discrete beat signal after ADC sampling. As illustrated in FIG. 4C, the shifting is expressed by the round-trip delay between the radar and the human object, which is approximated by equation (1) to t_{d}≈2d₀/c with c denoting the speed of light.

Relying on derivations from references [11]-[13] for the FMCW signal model, the continuous beat signal of the In-phase channel for a single chirp at a given frame, can be described as

$${s}_{b} ( t ) {=}^{Δ} {x}_{b} cos ( 2 π {f}_{b} t + {ψ}_{b} ( t ) ) , t ∈ [ \begin{matrix}{t}_{d} & {T}_{c}\end{matrix} ] , \qquad ( 2 )$$

with x_{b}, f_{b} and ψ_{b}(t) respectively denoting the amplitude, frequency and phase terms of the beat signal, due to the mixing process between the received and transmitted signals in the overlapping time interval [t_{d} T_{c}], where T_{c} is the duration of a single chirp, as illustrated in FIG. 4C.

The constant beat frequency is defined as

$${f}_{b} {=}^{Δ} {St}_{d} = \frac{2 S}{c} {d}_{0} , \qquad ( 3 )$$

where S≙B/T_{c} corresponds to the rate of the frequency sweep with B being the chirp's total bandwidth. The time-varying beat phase is:

$${ψ}_{b} ( t ) {=}^{Δ} \frac{4 π}{{λ}_{max}} ( {d}_{0} + v ( t ) ) , t ∈ [ \begin{matrix}{t}_{d} & {T}_{c}\end{matrix} ] , \qquad ( 4 )$$

where λₘₐₓ denotes the maximal wavelength of the chirp.

In practice, thoracic displacement is approximately constant with respect to a chirp's duration [11]. Hence, in order to extract the temporal variation of a human thorax, consecutive chirps are to be transmitted at intervals of Tₛ>>T_{c} seconds, denoted as the slow-time sampling interval of v(t).

Based on Eq. (4) and the above argument, a discrete phase signal over L given frames can be defined as:

$${ψ}_{b} [ l ] {=}^{Δ} \frac{4 π}{{λ}_{max}} ( {d}_{0} + v [ l ] ) , l = 1 , … , L , \qquad ( 5 )$$

where v[l]≙v(lTₛ).

Then, the continuous beat signal at each frame l, denoted by

$${\tilde{s}}_{b} ( t , {lT}_{s} ) = {x}_{b} cos ( 2 π {f}_{b} t + {ψ}_{b} [ l ] ) ,$$

is sampled by the ADC component at the sampling interval T_{f}, considered as the fast-time sampling period of \tilde{s}_{b}(t, lTₛ) (FIG. 4C, bottom). This yields the following 2-D discrete beat signal of the In-phase channel:

$${y}_{I} [ n , l ] {=}^{Δ} {\tilde{s}}_{b} ( {nT}_{f} , {lT}_{s} ) = {x}_{b} cos ( 2 π {f}_{b} {nT}_{f} + {ψ}_{b} [ l ] ) , \qquad ( 6 )$$

where n=1, . . . , N and l=1, . . . , L.

The discrete beat signal obtained by the parallel Quadrature channel, which is a 90° shifted version of Eq. (6), is used to compose the following complex exponential term:

$$y [ n , l ] {=}^{Δ} {\tilde{x}}_{b} exp ( j ( 2 π {f}_{b} {nT}_{f} + {ψ}_{b} [ l ] ) ) , \{ \begin{matrix}n = 1 , … , N \\ l = 1 , … , L\end{matrix} . \qquad ( 7 )$$

The samples in Eq. (7) form a 2-D measurement matrix Y∈ [symbol] ^{N×L} where Y(n, l)=y[n, l]. The samples along the row dimension are referred to as the fast-time samples and are related to the distance of the human object d₀ to the radar for the l'th frame via Eq. (3). The samples along the column dimension of Eq. (7) are referred to as the slow-time samples and are associated with the human thoracic vibration function v[l], which is reflected in varying phase values Eq. (5) between successive frames.

Thus, by estimating f_{b} and {ψ_{b}[l]}ₗ₌₁^{L}, it is possible to respectively evaluate do and {v[l]}ₗ₌₁^{L} by relations Eq. (3) and Eq. (5), from which various methods can be used to extract the corresponding RR and HR at the given time window.

FIG. 5 exemplifies the NCVSM system operation for monitoring multiple individuals. In the figure: the right side of the figure, i.e., blocks 1.a to 1.e, describes the method of yielding accurate estimates of respiration rate (RR) and heart rate (HR) by exploiting the sparse composition of the input data in accordance with human cardiopulmonary properties and using only a single channel; and left side of the figure, blocks 2.a to 2.g, describes the conventional framework which lacks adequate theoretical explanations and presents difficulties in dealing with noisy, cluttered environments. With the help of the left diagram of FIG. 5, the existing processing techniques for NCVSM based on the standard FMCW model presented in Eq. (7) are reviewed.

First, the raw signals received from the I and Q channels are pre-processed to construct the matrix Y (FIG. 5, block 2.a). Next, to locate the humans in the radar's FOV and their Doppler information, the Range vs. Slow-Time map [11] is determined/computed by performing a row-wise fast-time FFT over the complex representation of Y, and converting the fast-time axis to a distance-based one, using Eq. (3) (FIG. 5, block 2.b).

Two widely used spectrum-based methods for identifying human-related range bins from the given map (FIG. 5, block 2.c) are the manual localization [9] and the Maximum Average Power methods. The motivation for exploiting the map's magnitudes stems from Eq. (3) and Eq. (7), but since the model in Eq. (7) does not address the presence of clutters, it can lead to a selection of high-intensity disturbances instead of proper localization of the people to be monitored. Sacco et al. [10] used the std estimate to distinguish between human and static objects. However, since std does not exploit characteristics of human thoracic motion, a general search for vibrations may result in an incorrect selection of other vibrating objects, such as fans.

Since the human Doppler information is modulated in the phase of a complex exponential wave Eq. (5), Eq. (7), once the correct vectors are located, a phase extraction process is performed to estimate {ψ_{b}[l]}ₗ₌₁^{L} of the corresponding human (FIG. 5, block 2.e). Traditionally, phase extraction is carried out by the arctangent demodulation algorithm, followed by an unwrapping procedure. It should be noted that to compensate for the bounds restriction of the arctangent function, the known techniques of references [12] and [13] used the extended differentiate and cross-multiply (DACM) algorithm which is based on the derivative of the arctangent function. However, it is highly sensitive to noise and computationally heavy. To improve the extraction quality due to the non-linearity of the operators, the techniques of references [10] and [11] performed correction algorithms for DC component offset, prior to this step (FIG. 5, block 2.d). Though, as will be described below, this operation is not required with proper processing.

Given the extracted phase terms, where each contains an estimate of human thoracic vibration {v[l]}ₗ₌₁^{L} via Eq. (5), frequency estimation techniques are used to find the desired rates of respiration and heartbeat. The most common method is to apply the FFT algorithm to the given samples, relying on the fact that, at rest, the frequency bands of heartbeat and respiration are usually distinct [8]-[12]. Particularly, the known techniques of references [8], [11] and [12] used band-pass filters to separate the domains and enhance the relatively weak heartbeat signal.

Aiming to improve frequency resolution and to avoid spectral leakage, the technique of reference [15] performed zero-padding prior to the slow-time FFT. Although extremely fine FFT resolution can be obtained this way, two coarsely separated frequencies cannot be resolved. In contrast, Adib et al. [8] performed linear regression on the phase of a filtered complex time-domain signal, to obtain more precise measurements. The latter improves the accuracy of the estimates, however, the maximal peak is limited by the underlying frequency grid of the DFT. Finally, the technique of reference [16] used the MUSIC algorithm, to estimate the vital frequencies. However, this approach is sensitive to a low signal-to-noise-ratio (SNR) and suffers from high computational complexity since it requires explicit eigenvalue decomposition of an autocorrelation matrix, followed by a linear search over a large space [21].

In the technique of the present disclosure, simultaneous vital signs monitoring is performed for several people located at different radial distances from the radar, in a realistic environment that contains clutter and noise. To this end, the inventors developed an extension to the model in Eq. (7) for FMCW radar signals addressing NCVSM of multiple people, using only a single channel and a SISO configuration.

In the following, the details of the sparsity-based methodology of the present disclosure using this model are described.

Observing the model in Eq. (7), one sees that a monitored individual is characterized by frequency and phase terms of a complex wave, that respectively correspond to the individual's distance from the radar Eq. (3) and his/her thoracic motion pattern Eq. (5), for L given frames. In the case of multiple individuals and clutter, each object in the radar's FOV, whether vibrating or stationary, can be modelled based on Eq. (7) using an appropriate beat frequency and phase. Consequently, the measured FMCW signal in this case includes a combination of several signal reflections. To this end, the inventors extend the signal model in Eq. (7) to

$$y [ n , l ] = {\sum }_{m = 1}^{M} {x}_{m} exp ( j ( 2 π {f}_{m} {nT}_{f} + {ψ}_{m} [ l ] ) ) + w [ n , l ] , \qquad ( 8 )$$

where n=1, . . . , N, l=1, . . . , L, and {w[n, l]} is a 2-D sequence of zero mean white complex Gaussian noise with some variance σ². Therefore, the received 2-D beat signal y[n, l] is composed of M≤N components (where M is the number of objects) where the triplets {xₘ, fₘ, ψₘ[l]} denote the amplitude, frequency and phase of each m'th complex wave (belonging to the m'th object).

The frequency fₘ of each object is proportional to its radial distance from the radar, dₘ, by

$${f}_{m} {=}^{Δ} \frac{2 S}{c} {d}_{m} , m = 1 , … , M . \qquad ( 9 )$$

It is noted that each frequency is distinct, which complies with the SISO limitation that allows for only a single object detection at any radial distance from the radar. The slow-time varying phase ψₘ[l] of each component is given by

$${ψ}_{m} [ l ] {=}^{△} Δ \frac{4 π}{{λ}_{max}} ( {d}_{m} + {v}_{m} [ l ] ) , l = 1 , … , L , \qquad ( 10 )$$

where the vibration function vₘ[l] is generally modeled for both human and clutter objects by

$${v}_{m} [ l ] {=}^{△} {\sum }_{q = 1}^{Q} {a}_{m , q} cos ( 2 π {g}_{m , q} {lT}_{s} ) , l = 1 , … , L . \qquad ( 11 )$$

The pairs {a_{m,q}, g_{m,q}}_{q=1}^{Q} are the corresponding amplitudes and frequencies, with the latter being limited by the slow-time frame rate fₛ≙1/Tₛ according to

$${\{ {g}_{m , q} \}}_{q = 1}^{Q} ∈ [ \begin{matrix}0 & {f}_{s} / 2\end{matrix} ) , for m = 1 , … , M .$$

It is known to use a sum of 2 sines corresponding to the rates of respiration and heartbeat, at a given time window [9], [16].

Here, the vibration model in Eq. (11) is based on generic {a_{m,q}}_{q=1}^{Q} and {g_{m,q}}_{q=1}^{Q} sets, that allow for adequate representation of both static and vibrating objects, such as fans.

Particularly, for the case of K individuals located in the radar's FOV, let us assume that the frequency set {g_{m,q}}_{q=1}^{Q} includes their HR and RR, which are denoted by fₕ^{(k)} and fᵣ^{(k)}, respectively, for k=1, . . . , K. As shown further below, the objects can be distinguished by utilizing sparse properties of the input signal, within the frequency limits that characterize normal respiration and heartbeat.

Let us define the slow-time varying complex amplitude \tilde{x}m[l], for m=1, . . . , M, as

$${\tilde{x}}_{m} [ l ] {=}^{△} {x}_{m} exp ( j {ψ}_{m} [ l ] ) , l = 1 , … , L . \qquad ( 12 )$$

Then, extended signal model of Eq. (8) can be represented as

$$y [ n , l ] = {\sum }_{m = 1}^{M} {\tilde{x}}_{m} [ l ] exp ( j 2 π {f}_{m} {nT}_{f} ) + w [ n , l ] . \qquad ( 13 )$$

For each l=1, . . . , L, the inventors assemble the fast-time samples of y[n, l] into a vector, resulting in

$${y}_{l} = A {\tilde{x}}_{l} + {w}_{l} , l = 1 , … , L , \qquad ( 14 )$$

where yₗ≙[y[1, l], . . . , y[N, l]]^{T}∈ [symbol] ^{N}, A∈ [symbol] ^{N×M} is a Vandermonde matrix whose entries are given by A(n, m)≙exp(j2πfₘnTₛ), \tilde{x}ₗ≙[\tilde{x}₁[l], . . . , \tilde{x}_{M}[l]]^{T}∈ [symbol] ^{M}, and wₗ≙[w[1, l], . . . , w[N, l]]^{T}∈ [symbol] ^{N} is the noise vector.

In order to perform continuous NCVSM, the FMCW radar operates and generates data frames throughout the entire monitoring duration. To this end, at each predefined time interval Tᵢₙₜ, the sequence {yₗ}ᵢ₌₁^{L} Eq. (14) is formed by collecting the last L frames up to that point in time. The number, L, of frames to be processed is determined by a predefined time window T_{win} according to L=T_{win}fₛ, where the units of T_{win} and fₛ are [s] and [1/s], respectively. For convenience, the inventors reformulate the observations in Eq. (14) for each Tᵢₙₜ, using the following matrix form:

$$Y = A \tilde{X} + W , \qquad ( 15 )$$

where Y≙[y₁, . . . , y_{L}]∈ [symbol] ^{N×L} is the given measurement matrix, \tilde{X}≙[\tilde{x}ₗ, . . . , \tilde{x}_{L}]∈ [symbol] ^{M×L}, and W≙[w₁, . . . , w_{L}]∈ [symbol] ^{N×L} is the noise matrix. The above data model is assumed to have the following properties:
A-1: The sequence {wₗ}ₗ₌₁^{L} which forms the noise matrix W can be viewed as L independent and identically distributed realizations of a zero mean complex Gaussian noise vector with covariance matrix σ²I_{N}, where I_{N} denotes a size-N identity matrix; and
A-2: Only K<<M individuals are being monitored, where each is stationary except for minor movements caused by breathing or speaking, where the latter induces a row-wise sparsity in \tilde{X}, meaning that the vectors {\tilde{x}ₗ}ₗ₌₁^{L} share a joint support.

Based on the model in Eq. (15), the first goal is to recover the row-coordinates of \tilde{X} associated with humans in the radar's FOV, denoted by the support [symbol] , by which it is possible to estimate the human locations and filter the relevant Doppler information. The second goal, using the recovered [symbol] , is to continuously evaluate the RR and HR of each detected individual throughout the complete monitoring duration. Mathematically, every Tᵢₙₜ seconds the inventors seek to estimate the corresponding fᵣ^{(k)} and fₕ^{(k)}, for k=1, . . . , K.

Turning back to FIG. 5, with the aid of diagram on the right side of FIG. 5 (blocks 1.a to 1.e), describing the technique of the present disclosure, below each stage of the proposed sparsity-based approach is detailed, based on the model developed above.

Referring to the first stage of pre-processing shown in FIG. 5, block 1.a, at the start of each monitoring iteration determined by Tᵢₙₜ, a preliminary processing is performed to assemble Y according to Eq. (15) with an increased SNR.

Based on the assumption A-1 that each element of W is derived from a Gaussian distribution with variance σ², the slowness of thoracic motion relative to a single chirp [11] can be utilized to reduce the variance of each element, by averaging several data observations at each frame. To accomplish this, a transmission scheme is defined in which G>1 consecutive chirps are transmitted in each frame instead of a single one, with G being limited by the frame duration Tₛ, as illustrated in FIG. 4C.

For every Tᵢₙₜ, this process generates G beat signal duplicates per frame that differ only in the noise impact, i.e., arranging the input data similarly to Eq. (15) results in

$${Y}_{pre} = [ {\{ {y}_{1 , 9} \}}_{g = 1}^{G} , … , {\{ {y}_{L , g} \}}_{g = 1}^{G} ] ∈ {ℂ}^{N \times GL} .$$

By averaging the fast-time row samples every G columns, the inventors get Y=[y̅₁, . . . , y̅_{L}]∈ [symbol] ^{N×L}, where

$${\overline{y}}_{l} {=}^{Δ} \frac{1}{G} {\sum }_{g = 1}^{G} {y}_{l , g} ,$$

l=1, . . . , L. This procedure yields the model in Eq. (15) while reducing the noise variance of each data element by a factor of G, versus the use of a single chirp per frame.

Referring to FIG. 5, block 1.b where in the technique of the present disclosure the support of \tilde{X}, denoted [symbol] , is estimated, which allows to detect the radial distance of each human from the radar by Eq. (9), and to efficiently extract the corresponding Doppler samples for the remainder of the monitoring process, as detailed in the next step described below. Additionally, the inventors show that this localization can be performed only once in the entire monitoring period.

To this end, first, the fast-time frequencies {fₘ}ₘ₌₁^{M} (Eq. (9)) are assumed to lie on the Nyquist grid, i.e.,

$${f}_{m} = \frac{{f}_{ADC}}{N} {i}_{m} , {i}_{m} = 0 , … , M - 1 , \qquad ( 16 )$$

where f_{ADC}≙1/T_{f} is determined by the ADC component. It should be noted that using Eq. (9) and Eq. (16), the maximal detectable distance is

$${d}_{max} = \frac{{cf}_{ADC}}{2 SN} ( M - 1 ) .$$

Interestingly, since N is the number of fast-time samples, generally, M=N, although as detailed in further below, by selecting M=N/2 the model in Eq. (15) may be employed even using data from only a single channel, without jeopardizing estimation performance.

Second, the inventors denote by B^{(R)} and B^{(H)} the frequency bands of normal respiration and heartbeat, respectively, where B^{(R)}, B^{(H)}∈

$$[ \begin{matrix}0 & \frac{{f}_{s}}{2}\end{matrix} ) .$$

This prior knowledge of human-typical pulse and breathing frequencies aids in the separation of humans from static or vibrating clutter, such as fans. Hence, based on the slow-time frequency modulation structure of the vibration signal vₘ[l] Eq. 10, Eq. 11 in Eq. (8), spectral filtering of Y is performed in the slow-time axis, according to

$$\overline{Y} = \frac{1}{L} {( {F}_{L}^{H} ( Π ⊙ {F}_{L} {Y}^{T} ) )}^{T} , \qquad ( 17 )$$

where F_{L} is a full L-size DFT matrix, Π denotes an ideal window corresponding to the vital frequencies in B^{(H)}∪B^{(R)}, and ⊙ denotes the element-wise product.

Based on the assumption A-2 that X is a row-sparse matrix, inspired by [18], the inventors now recover it from Y̅ using a JSR technique formulated by the following optimization problem

$${min}_{\tilde{X} ∈ {ℂ}^{M \times L}} {‖ \overline{Y} - A \tilde{X} ‖}_{F}^{2} + λ {‖ \tilde{X} ‖}_{2 , 1} . \qquad ( 18 )$$

Here, to promote the row sparsity of \tilde{X}, the inventors use the regularization parameter λ≥0 and the mixed l_{2,1} norm defined by ∥X∥_{2,1}≙Σᵢ∥xⁱ∥₂, with xⁱ denoting the i'th row of a matrix X. The inventors solve Eq. (18) and find the support [symbol] using the fast iterative soft-thresholding algorithm (FISTA). The recovered support S is first used to calculate the distances of the monitored people from the radar through Eq. (9), that is, {dₘ}, for m∈ [symbol] . Then, since the technique deals with monitoring stationary subjects, the coordinates of support [symbol] are fixed throughout the monitoring, implying that one can recover [symbol] only once, and use it for all subsequent iterations. It should be noted that there are many other methods that solve JSR problems and that the above method is an exemplary one.

Referring to FIG. 5, block 1.c., the support [symbol] evaluated in the previous step allows to efficiently recover only the human-related Doppler samples of \tilde{X} given Y, throughout the remainder of the monitoring process.

Using support [symbol] and assumption A-2, the model in Eq. (15) can be written as

$$Y = {A}_{S} {\tilde{X}}_{S} + W , \qquad ( 19 )$$

with A [symbol] ∈ [symbol] ^{N×K} and \tilde{X} [symbol] ∈ [symbol] ^{K×L} respectively being the atoms of A and the rows of \tilde{X} corresponding to support [symbol] , where K=| [symbol] |. By knowing the support for each Tᵢₙₜ, the inventors can directly estimate [symbol] from Y, using the solution of the following Least-Squares (LS) problem

$${min}_{{\tilde{X}}_{𝒮} ∈ {ℂ}^{K \times L}} {‖ Y - {A}_{𝒮} {\tilde{X}}_{𝒮} ‖}_{F}^{2} \qquad ( 20 )$$

given by

$${\hat{X}}_{𝒮} = {( {A}_{𝒮}^{H} {A}_{𝒮} )}^{- 1} {A}_{𝒮}^{H} Y \qquad ( 21 )$$

Based on the Vandermonde structure of A defined above (Eq. (14)) and the condition of M that K<<M≤N, it can be considered that [symbol] As is invertible. Explicitly, [symbol] = [symbol] and [symbol] =NI_{K}, meaning that [symbol] in Eq. (21) can be represented as

$${\hat{X}}_{𝒮} = \frac{1}{N} {F}_{𝒮} Y , \qquad ( 22 )$$

where F [symbol] denotes a partial DFT matrix corresponding to the fast-time frequencies {fₘ} (Eq. (16)), for m∈ [symbol] .

It should be noted that by assuming that M=N/2, the frequencies {fₘ}ₘ₌₁^{M} (Eq. (16)) correspond to the positive tones of the sinusoidal combination Σₘ₌₁^{M}xₘ cos(2πfₘnT_{f}+ψₘ[l]), which would have been obtained in Eq. (8) when using the In-Phase channel solely. Hence, for M=N/2, the estimator in Eq. (22) when using both the I and Q channels is equivalent to that obtained from the use of only a single channel (I or Q), up to a constant factor. To avoid hardware overload and potential issues of using both channels, the inventors assume here that M=N/2 and use only the In-Phase channel.

As opposed to existing methods that compute the entire Range vs. Slow-Time map (FIG. 5, block 2.b), which is equivalent to replacing [symbol] in Eq. (22) with F_{M} that corresponds to all M=N/2 frequencies in Eq. (16), at each iteration the estimator used in the technique of the present disclosure recovers only the relevant DFT samples, which is considerably more efficient since | [symbol] |=K<<M.

Finally, since there are no humans within dₘ=0 of the radar, the estimator in Eq. (22) is always filtering the DC component, corresponding to m=1 in Eq. (16). As a result, a mechanism for DC offset correction is not included in the technique of the present disclosure, unlike other techniques (FIG. 5, block 2.d).

Using relation Eq. (12) and the definition of \tilde{X} above (Eq. (15)), it follows that

$${\tilde{X}}_{𝒮} ( m , l ) = {x}_{m} exp ( j {ψ}_{m} [ l ] ) , m ∈ 𝒮 . \qquad ( 23 )$$

That is, [symbol] Eq. (22) estimates the slow-time varying phasor terms associated with humans in the radar's FOV. In order to estimate the appropriate vital signs (e.g., RR and HR from the thoracic vibrations of each individual, i.e., {fᵣ^{(k)}, fₕ^{(k)}}ₖ₌₁^{K} from {vₘ[l]}ₗ₌₁^{L}, m∈ [symbol] , an approximation of the phase terms {ψₘ[l]}ₗ₌₁^{L}, m∈ [symbol] (Eq. (10)) is first extracted from [symbol] (FIG. 5, block 1.d). Hence, the following element-wise angle extraction operation on [symbol] is performed, which yields the following L×K vibration matrix

$$V ( l , k ) \hat{=} unwrap {( ∠ ( {\hat{X}}_{S} ( 𝒮 \{ k \} , l ) ) )}^{T} , \{ \begin{matrix}k = 1 , … , K \\ l = 1 , … , L\end{matrix} \qquad ( 24 )$$

where unwrap (⋅) denotes the unwrapping procedure based on reference [11], used since the unambiguous phase range is limited by (−ππ]. The angle extraction operator ∠(⋅) is based on the four-quadrant arctangent function applied with Matlab's ‘a tan 2.m” function.

Referring to FIG. 5, block 1.e, the VSDR method is selected for estimating RR and HR out of many frequency estimation methods. In the final stage of each iteration, both the RR and HR of each individual, {fᵣ^{(k)}, fₕ^{(k)}}ₖ₌₁^{K} are estimated given the vibration matrix V, and recorded at the corresponding time instant defined by Tᵢₙₜ.

As described below, the inventors develop a procedure for selecting high-resolution estimates of vital signs out of two unique dictionaries based on human-typical pulse and breathing frequencies.

First, using Eq. (10) and Eq. (11), the matrix V in Eq. (24) can be viewed as a chain of vectors corresponding to the detected humans' thoracic vibration signals, i.e.,

$$V = [ {v}_{1} , … , {v}_{K} ] , \qquad ( 25 )$$

$$with$$

$${v}_{k} = {Da}_{k} + {n}_{k} , k = 1 , … , K , \qquad ( 26 )$$

where each vector aₖ∈ [symbol] ^{Q} includes Q amplitudes {ã_{q}^{(k)}}_{q=1}^{Q} and D∈ [symbol] ^{L×Q} is a cosine-based dictionary matrix with entries D(l, q)≙cos(2π\tilde{g}_{q}lTₛ) where the frequencies {\tilde{g}_{q}}_{q=1}^{Q}∈[0 fₛ/2). Finally, {nₖ}ₖ₌₁^{Q} denotes a sequence of length-L independent and identically distributed noise vectors as a result of the non-linear operations in Eq. (24).

Ideally, to achieve optimal frequency resolution, one uses T_{win}=60 seconds which corresponds to the number of heartbeats or breath cycles per minute definition, i.e., bpm. However, this comes at the expense of reduced temporal localization. Therefore, to allow for smaller time windows but with increased resolution the inventors uniformly divide the segment [0 fₛ/2) according to a resolution of 1 bpm, i.e., the frequencies {\tilde{g}_{q}}_{q=1}^{Q} satisfy the condition:

$${\tilde{g}}_{q} = {h}_{q} \frac{{f}_{s}}{Q} , {h}_{q} = 0 , … , \frac{Q}{2} - 1 , Q = 60 {f}_{s} . \qquad ( 27 )$$

The inventors assume that for every T_{win} the two most dominant frequencies of each thoracic vibration vₖ, are the rates of heartbeat and respiration. However, the amplitude of the heart signal is much smaller than that of respiration, so in order to facilitate its detection, the inventors utilize the phenomenon that at rest, the frequency bands are usually separated from each other. It should be noted that unlike teachings of references [8], [11] and [12], the inventors do not perform a signal separation procedure prior to this stage (FIG. 5, block 2.f). Here, the inventors exploit this band-separation property to effectively focus the frequency search on limited dictionaries corresponding to each band.

Explicitly, the inventors define the vital signs based dictionaries D^{(R)}∈ [symbol] ^{L×Q}^{_{R}} and D^{(H)}∈ [symbol] ^{L×Q}^{_{H}} as follows

$${D}^{( R )} ( l , q ) \hat{=} cos ( 2 π {\tilde{g}}_{q}^{( R )} {lT}_{s} ) and \qquad ( 28 )$$

$${D}^{( H )} ( l , q ) \hat{=} cos ( 2 π {\tilde{g}}_{q}^{( H )} {lT}_{s} ) ,$$

where both frequencies {\tilde{g}_{q}^{(R)}} and {\tilde{g}_{q}^{(H)}} constitute a subset of Eq. (27), satisfying {\tilde{g}_{q}^{(R)}}∈B^{(R)} and {\tilde{g}_{q}^{(H)}}∈B^{(H)}.

Using the notation from Eq. (28) and the assumption that only the vital frequency bands B^{(R)} and B^{(H)} contribute to the thoracic vibration of each vₖ, it is possible to represent the model in Eq. (26) as

$${v}_{k} = {D}^{( R )} {a}_{k}^{( R )} + {D}^{( H )} {a}_{k}^{( H )} + {n}_{k} , k = 1 , … , K , \qquad ( 29 )$$

where aₖ^{(R)}∈ [symbol] ^{Q}^{_{R}} and aₖ^{(H)}∈ [symbol] ^{Q}^{_{H}} are both amplitude vectors with only a single non-zero element whose coordinate indicates the corresponding rate (RR/HR), of the k-th human. To recover aₖ^{(R)} and aₖ^{(H)} from each vₖ in Eq. (29), the inventors apply the following estimators:

$${\hat{a}}_{k}^{( R )} = {D}^{( R ) T} {v}_{k} and {\hat{a}}_{k}^{( H )} = {D}^{( H ) T} {v}_{k} , \qquad ( 30 )$$

∀ₖ=1, . . . , K. The maximum's coordinate of âₖ^{(R)} and âₖ^{(H)} points to the RR estimation \hat{f}ᵣ^{(k)} and the HR estimation \hat{f}ₕ^{(k)}, from {\tilde{g}_{q}^{(R)}} and {\tilde{g}_{q}^{(H)}}, respectively. To enhance estimation stability, the inventors replace the computed \hat{f}ᵣ^{(k)} and \hat{f}ₕ^{(k)} with the average of the estimates from the last 3 and 1.5 seconds, respectively. This vital signs-based dictionary recovery is referred to as the VSDR method.

Data processing (Process 1) presented below summarizes the example of the technique of the present disclosure with Lₗᵢₚ and Iₘₐₓ being respectively the Lipschitz constant and the maximal number of iterations in the FISTA algorithm, and the set of all measurement matrices Eq. (15) being processed during the monitoring is denoted by {Y}.

In the following, the performance of the proposed exemplary method is evaluated and compared to existing techniques, using a simulation that combines the measurement model in Eq. (15) with real Electrocardiography (ECG) impedance data of 30 participants from reference [19].

It should be noted that Process 1 is divided such that during the first monitoring iteration, a localization procedure is performed, after which the vital signs of the detected people are estimated throughout the rest of the monitoring period. As a result, two simulation studies are presented here. The first simulation investigates the multiple-people localization part in a clutter-rich environment, while the second examines NCVSM given human thoracic vibrations from the previous study.

Process 1 - Sparsity Based NCVSM of Multiple People:

```
Input: Tᵢₙₜ, {Y}, A, λ ≥ 0, Lₗᵢₚ, Iₘₐₓ, D^{(R)}, D^{(H)}.
At each Tᵢₙₜ , perform the following:
  First iteration:
  1: Pre-process Y (as described above - block 1.a in Fig. 5)
  2: Filter spatial interference (spectral filtering by Eq. (17)) to obtain Y̅
  3: Perform JSR by Eq. (18) using FISTA and save the obtained
  support [symbol]
  4: Compute the distances {dₘ}, m ∈ [symbol]   using Eq. (9)
  5: Recover \hat{X} [symbol]   for obtained [symbol]   and Y by Eq. (22)
  6: Extract V from \hat{X} [symbol]   according to Eq. (24)
  7: Estimate {âₖ^{(R)}, âₖ^{(H)}}ₖ₌₁^{K} for extracted V by VSDR of Eq. (30)
Output: \hat{f}ᵣ^{(k)} and \hat{f}ₕ^{(k)}, ∀k = 1, ... , K
In all other iterations:
  1: Pre-process Y and skip to steps 5-7 above using support [symbol]
Output: \hat{f}ᵣ^{(k)} and \hat{f}ₕ^{(k)}, ∀k = 1, ... , K
```

To this end, the inventors used relations Eqs. (8)-(11) that form the model in Eq. (15), to compose seven different objects in the radar's FOV (of which K=3 humans), each characterized by a corresponding value of xₘ being the amplitude of the m'th complex wave belonging to the m'th object) (see Eq. (8)), radial distance dₘ of the m'th individual to the radar (see Eq. (9)) and the respective vibration function {vₘ[l]}ₗ₌₁^{L} (Eq. (11)), as detailed in Table 1 below.

A schematic illustration of the experiment can be seen in the above-described

### FIG. 4B.

To create a realistic environment, as shown in Table 1, the inventors adjusted each xₘ so that static objects have the strongest reflections, as compared to fans, and finally humans, all of which fade as a function of the radial distance dₘ. Furthermore, to examine the impact of environmental noise, the inventors used an SNR term that controls the variance of {w[n, l]} Eq. (8) via SNR≙1/σ². As to the monitoring, the inventors determined that the humans would be monitored simultaneously for 10 minutes, with RR and HR estimates computed during every time interval Tᵢₙₜ=0.05 [s], using In-phase channel data collected from the last time window T_{win}=30 [s], starting at T_{win}.

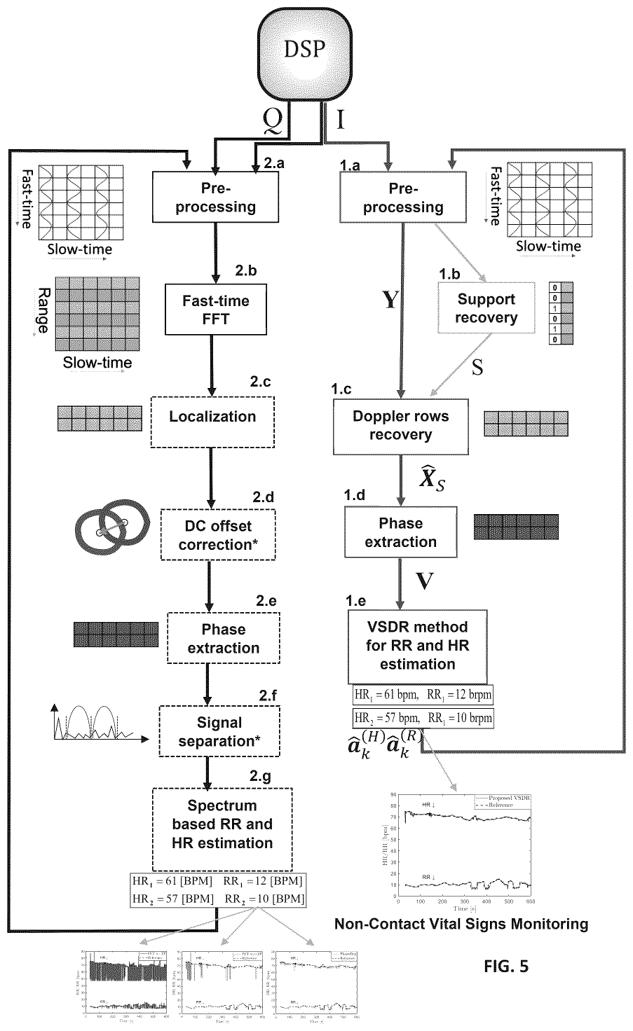

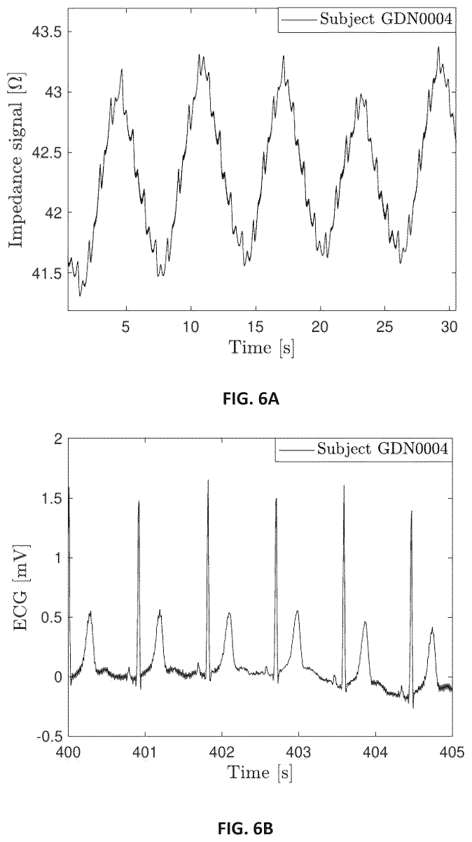

Reference is made to FIGS. 6A to 6B which show, respectively, real impedance and ECG data from ref. [19], wherein FIG. 6A shows impedance [Ω] used as a reference for comparing the RR estimates and to simulate human thoracic vibration; and FIG. 6B shows ECG [mV] used as a reference for comparing the HR estimates.

To reliably simulate prolonged human breathing, the inventors used data from the resting scenario of reference [19], in which the participants were lying on a table wired to several monitoring devices and were told to breath calm and avoid large movements for at least 10 minutes. In the analysis, the inventors selected and rescaled the 100 [Hz] impedance signal “tfm_z0”, which provides insight into the impedance change of the thorax, to simulate human thoracic vibrations over 10 minutes of monitoring, as shown in FIG. 6A and implemented in Table 1 below. A variety of cardiac and respiratory parameters can be extracted from the impedance signal, including RR and HR, thus the raw signal serves as a reference for comparing the RR estimation results. As to the HR reference, the inventors used the gold-standard 2000 [Hz] ECG signal “tfm_ecg1” (FIG. 6B), and down-sampled it to 100 [Hz] to correspond to Tₛ=10 [ms]. The main FMCW radar parameters for assembling the model in Eq. (15) are based on Texas Instruments IWR1642 76 to 81 [GHz] mmWave sensor and summarized in Table 2.

This study examined the localization of several people in a clutter-rich environment given the pre-processed Y of the first iteration, as a preliminary step to monitor their vital signs.

TABLE 1

| Object type | xₘ | dₘ | {vₘ[l]ₗ₌₁^{L} |
|---|---|---|---|
| Vibrating fan #1 | 0.7 | 1.5 | 0.1 cos(2π40lTₛ), l = 1, . . . , L |
| Human #1 | 0.5 | 2 | Impedance data from [19] |
| Static clutter #1 | 1 | 2.3 | 0, ∀l = 1, . . . , L |
| Human #2 | 0.45 | 2.6 | Impedance data from [19] |
| Static clutter #2 | 0.9 | 2.9 | 0, ∀l = 1, . . . , L |
| Vibrating fan #2 | 0.6 | 3.1 | 0.1 cos(2π40lTₛ), l = 1, . . . , L |
| Human #3 | 0.4 | 3.5 | Impedance data from [19] |

TABLE 2

| Parameter | Symbol | Value |
|---|---|---|
| Maximal chirp wavelength | λₘₐₓ | 3.9[mm] |
| Chirp duration | T_{c} | 57[μs] |
| ADC sampling rate | ƒ_{ADC} | 4[MHz] |
| Rate of frequency sweep | S | 70[MHz/μs] |
| Frame duration | Tₛ | 10[ms] |
| # fast-time samples | N | 200 |
| # of chirps per frame | G | 150 |

The parameters of the proposed JSR localization technique were set as follows. The vital slow-time frequencies of Π in Eq. (17) were drawn from the Nyquist grid of length L determined by fₛ. The parameters for solving Eq. (18) using FISTA were set to λ=30, Lₗᵢₚ=4.5e6 (Lipschitz constant) and Iₘₐₓ=1000 (the maximal number of iterations in the FISTA algorithm). Finally, the thoracic vibrations selected here for humans in Table 1, were based on resting impedance data of subjects 1-3 from reference [19].

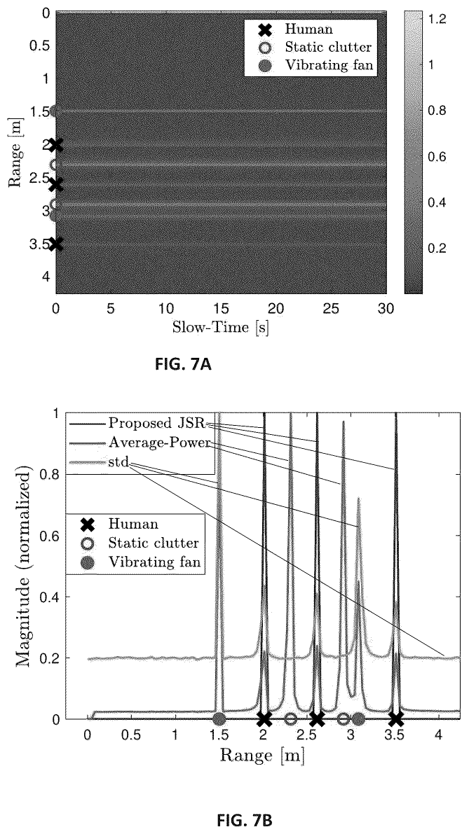

Reference is made to FIGS. 7A to 7B which exemplify multiple people localization by the setup of Table 1, for SNR=0 [dB]. Here, FIG. 7A shows the Range vs. Slow-Time map via the magnitudes of \hat{X}_{M}. The row-wise intensities correspond to the DC component and the reflections of objects of Table 1. FIG. 7B compares several known localization techniques with the technique of the present disclosure.

By replacing [symbol] in Eq. (22) with F_{M}, M=N/2, the estimator in Eq. (22) evaluates the complete Range vs. Slow-Time map by

$${\hat{X}}_{M} = \frac{1}{N}$$

F_{M}Y and adjusting the fast-time axis by Eq. (9). FIG. 7A depicts the Range vs. Slow-Time map via the magnitudes of \hat{X}_{M}, for SNR=0 [dB]. The visible row-wise intensities correspond to the DC component as well as the reflections from objects listed in Table 1. It should be noted that the technique of the present disclosure, contrary to the standard practice of using this map for localizing the humans (FIG. 5: block 2.c, references [9]-[11]), has no such need/requirement.

One can observe in FIG. 7B that the proposed JSR method indicates the correct locations of the humans compared to the intensity-based Maximum Average Power method [11] and the std approach [10]. It should be noted that the former technique (Maximum Average Power method) inadvertently selects the strong reflections obtained from static clutters, whereas the latter (std approach) seeks for highly oscillating objects and thus incorrectly selects the vibrating fans over humans. In contrast to the compared localization techniques, the technique of the present disclosure utilizes both the characteristics of human-typical vital frequencies and the prior knowledge of the sparse structure of matrix \tilde{X}.

Given successful human localization from the above-described simulations, the inventors examined the performance of VSDR (Eq. (30)) for NCVSM and compared it to other state-of-the-art techniques using data of 30 individuals from the resting scenario in reference [19].

To ensure a fair comparison of various techniques, the inventors applied the first iteration of Process 1 described above assuming that all considered humans were successfully identified. Then, the inventors only compared the last step (7) of said process, in which the human vital signs are estimated given the extracted matrix V. To facilitate the analysis, the thoracic vibration v_{z} (Eq. (25)) of human #2 from Table 1 is studied here, using impedance data from all 30 participants in reference [19]. Similar findings are obtained for the remaining vibrations. In addition, the frequency bands of respiration and heartbeat were set for all methods to B^{(R)}=[0.1 0.4][Hz] and B^{(H)}=[0.78 1.67] [Hz], respectively, corresponding to a normal resting state. It should be noted that all the procedures and parameter settings that preceded this step are the same among all methods.

The proposed VSDR of the technique of the present disclosure was first compared to the method detailed in reference [8] for estimating RR and HR given the phase of an FMCW signal, called here Phase-Reg. Moreover, VSDR was compared to an FFT-based peak selection in each frequency band, with zero-padding (FFT w/ZP as per reference [15]) and without (FFT w/o ZP as per references [8]-[12]). The padding of FFT w/ZP was set to fit a 60-second time window corresponding to frequency resolution of 1 [bpm]. All methods were implemented using MATLAB. As to the reference data, the inventors found that it is common to estimate the RR and HR of the reference signals via the DFT spectrum [8]-[11]. Assuming that both the ECG and impedance references are noise-free, they were padded similarly to FFT w/ZP, for optimal results.

To evaluate the monitoring accuracy, the inventors used several statistical metrics, based on the RR and HR estimates of the compared methods with respect to those of the references. The metrics used are the following: (i) SuccessRate, defined here as the percentage of time in which the estimate was different from the reference output by less than 2 [bpm]; (ii) Pearson Correlation Coefficient (PCC), showing results in the range [0 1]; (iii) Mean-Absolute Error (MAE); and (iv) Root-Mean-Square Error (RMSE).

The Success-Rate can predict the percentage of time an estimation error greater than 2 [bpm] is expected, the PCC measures the linear correlation between the two data sets, and while in the MAE metric, each error contributes in proportion to its absolute value, the RMSE is a well-known metric that emphasizes the occurrence of coarse errors.

The inventors investigate various SNR cases, each of which involves monitoring data of 30 individuals from reference [19]. The performance score produced for each metric, given SNR, is taken as the median across all 30 participants. It is noted that 10 minutes of monitoring starting at T_{win}=30 [s], with output obtained at each Tᵢₙₜ=0.05 [s] brings to 11400 estimates for comparison to the references, for each participant.

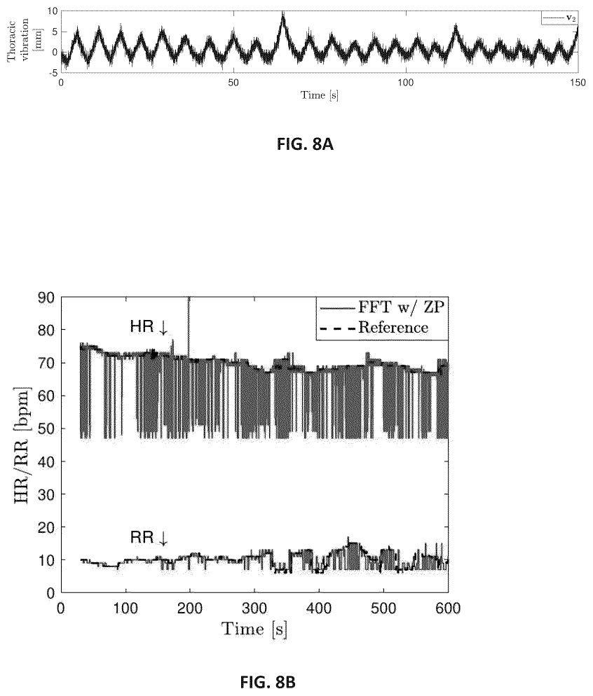

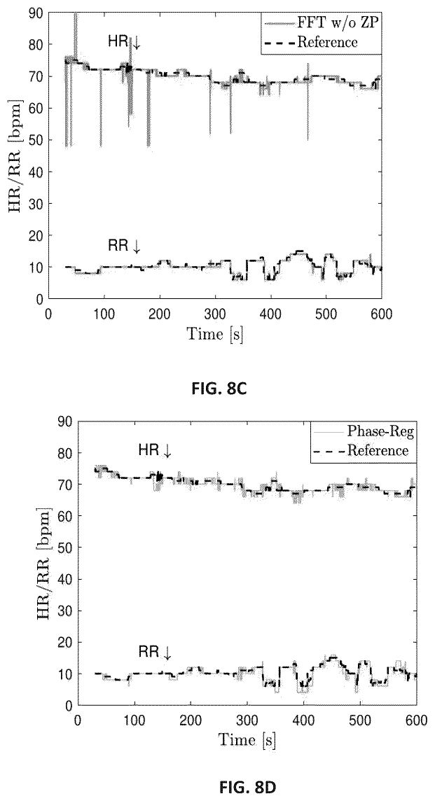

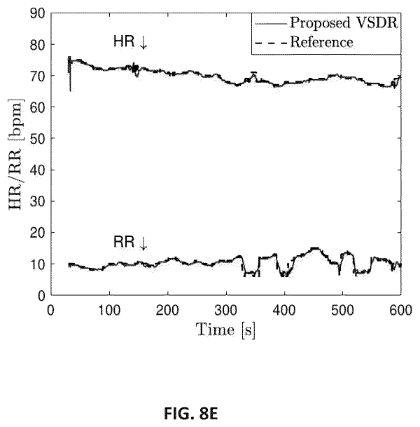

Reference is made to FIGS. 8A to 8E which exemplify the performance of NCVSM based on data of subject GDN0004 from reference [19] for SNR=1 [dB], compared to the references, using VSDR of the present disclosure and other state-of-the-art techniques. FIG. 8A shows the extracted thoracic vibration v₂ of human #2 from

Table 1; FIG. 8B shows results of FFT-based peak selection with zero-padding; FIG. 8C shows results of FFT-based peak selection without zero-padding; FIG. 8D shows results by the method detailed in reference [8] called here Phase-Reg; and FIG. 8E shows results by the VSDR (Vital Signs based Dictionary Recovery) method of the present disclosure.

FIG. 8A depicts the extracted thoracic vibration pattern v_{z} corresponding to subject GDN0004 for the case of SNR=1 [dB]. FIGS. 8B to 8E show NCVSM compared to the references, by VSDR and the other examined techniques, given the extracted vibration v₂ at each Tᵢₙₜ. It can be seen how both the HR and RR estimates by the proposed VSDR technique show great resemblance to those of the reference, compared to the other techniques in which the noisy setup impairs the evaluations.

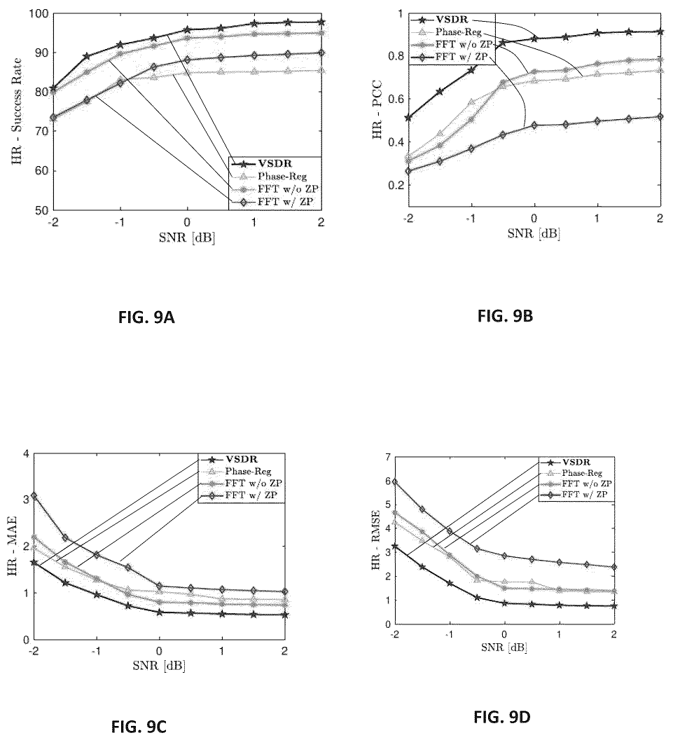

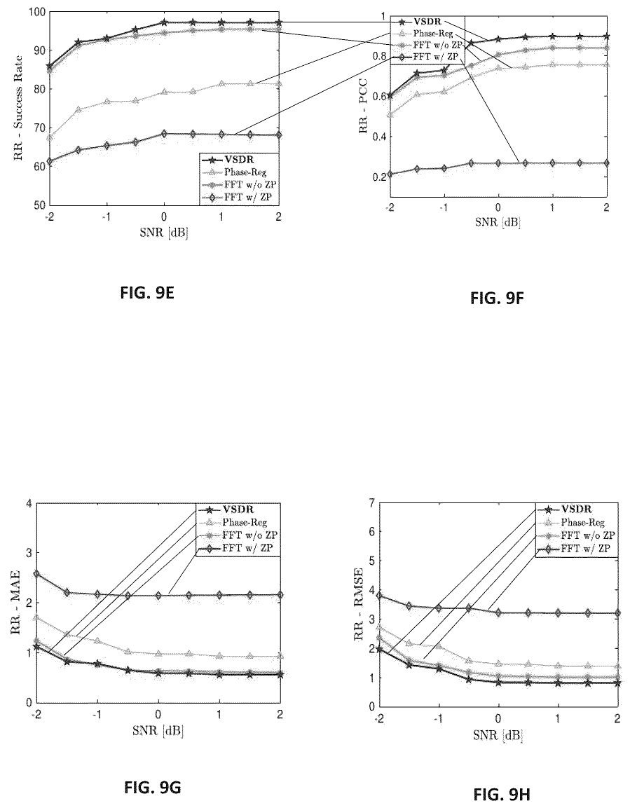

FIGS. 9A to 9H show the Success-Rate, PCC, MAE and RMSE for both HR and RR estimation by all examined methods, as a function of the SNR. One sees that VSDR outperforms the other compared methods in all 4 metrics, for every SNR value. Also, note the considerable difference in performance in the more challenging task of HR monitoring due to the weak heartbeat signature, in favor of the proposed approach. As for the RR monitoring case, the small difference between VSDR and the popular FFT w/o ZP can be explained by the relative dominance of the respiratory signal that facilitates its detection, bringing to fine results in both techniques. Detailed median accuracy scores for the noisy case of SNR=0 [dB] can be found in Tables 3 and 4 below.

The performance advantage is a consequence of the property that, unlike all other compared methods for NCVSM given a thoracic vibration, the proposed VSDR employs a frequency search over high resolution grids corresponding to human-typical cardiopulmonary frequencies via a dictionary-based approach (Eq. (30)).

TABLE 3

| | Success- | | | |
|---|---|---|---|---|
| Method | Rate [%] | PCC | MAE | RMSE |
| FFT w/ ZP | 88.06 | 0.47 | 1.14 | 2.84 |
| FFT w/o ZP | 93.56 | 0.72 | 0.79 | 1.48 |
| Phase-Reg | 84.76 | 0.68 | 1.02 | 1.76 |
| Proposed VSDR | 95.68 | 0.87 | 0.57 | 0.85 |

TABLE 4

| | Success- | | | |
|---|---|---|---|---|
| Method | Rate [%] | PCC | MAE | RMSE |
| FFT w/ ZP | 68.37 | 0.26 | 2.13 | 3.20 |
| FFT w/o ZP | 94.32 | 0.80 | 0.63 | 1.03 |
| Phase-Reg | 79.05 | 0.73 | 0.97 | 1.45 |
| Proposed VSDR | 97.04 | 0.88 | 0.58 | 0.81 |

In another aspect of the present disclosure, the inventors developed an extended mathematical signal model for the problem of NCVSM of multiple people in a cluttered scenario, using SIMO FMCW radar (as opposed to SISO described in detail above). Based on the sparse representation of people via this model, the inventors propose a human spatial localization using a joint-sparse recovery (JSR) mechanism [18]. This approach estimates both radial distance and azimuth angle and allows for computationally efficient extraction of Doppler information throughout the complete monitoring process. Then, high-resolution NCVSM of multiple people using the Vital Signs based Dictionary Recovery (VSDR) technique described above is demonstrated. The performance of the proposed methodology is verified through simulations that incorporate the developed model with in vivo data of 3 monitored individuals from reference [19]. The results demonstrate accurate human localization in a multiple-object scenario as well as precise NCVSM of multiple people, when compared to state-of-the-art techniques based on
[8-12,15], using several statistical metrics.

A typical linear FMCW radar transmits a saw-tooth waveform at each given time frame, called chirp, whose frequency linearly increases over time. The reflected echo signals are mixed with versions of the transmitted ones to obtain analog base-band signals, called beat signals [11,15]. For each frame, the beat signals are sequentially sampled by the ADC, resulting in discrete signals of length N.

The inventors consider a SIMO Uniform Linear Array (ULA) with a single transmitter and K receivers spaced by rₖ≙(k−1)λ/2, k=1, . . . , K where λ denotes the chirp's maximal wavelength. To that end, the inventors extend the single-input-single-output (SISO) signal model described above for L given frames of N beat samples using K>1 receivers (instead of K=1 for the SISO signal model), to the following 3-D discrete beat signal based on P×M objects in the radar's FOV (P being the number of azimuth angles and M being the number of radial distances):

$$y [ n , k , l ] = {\sum }_{p = 1}^{P} {\sum }_{m = 1}^{M} {x}_{m , p} {e}^{j ( 2 π {f}_{m} {nT}_{f} + {ψ}_{m , p} [ l ] + {ϕ}_{p} [ k ] )} + w [ n , k , l ] \qquad ( 31 )$$

where n=1, . . . , N, k=1, . . . , K, l=1, . . . , L and {w[n, k, l]} is a 3-D sequence of zero mean independent and identically distributed complex Gaussian noise with variance σ². Here, T_{f} denotes the ADC sampling interval and x_{m,p} denotes the received amplitude of the {m, p}'th object based on its radar cross section (RCS).

Each frequency fₘ is distinct and proportional to a different radial distance from the radar dₘ:

$${f}_{m} \hat{=} \frac{2 S}{c} {d}_{m} , m = 1 , … , M \leq N \qquad ( 32 )$$

where c denotes the speed of light and S≙B/T_{c} corresponds to the rate of the frequency sweep of each chirp, with B and T_{c} respectively being the chirp's total bandwidth and duration. The slow-time varying phase ψ_{m,p}[l] of each component is given by

$${ψ}_{m , p} [ l ] \hat{=} \frac{4 π}{λ} ( {d}_{m} + {v}_{m , p} [ l ] ) , l = 1 , … , L \qquad ( 33 )$$

where the function v_{m,p}[l] refers to small vibrations caused by human thoracic displacements or other vibrating objects, such as fans, and is therefore generally modeled as follows:

$${v}_{m , p} [ l ] \hat{=} {\sum }_{q = 1}^{Q} {a}_{m , p , q} cos ( 2 π {g}_{m , p , q} {lT}_{s} ) , l = 1 , … , L , \qquad ( 34 )$$

with the pairs {a_{m,p,q}, g_{m,p,q}}_{q=1}^{Q} denoting the corresponding amplitudes and frequencies and Tₛ≙1/fₛ is the frame duration, also known as the slow-time sampling interval. The generic vibration model in Eq. (34) allows for adequate representation of both static and vibrating objects, using appropriate values for {a_{m,p,q}}_{q=1}^{Q} and {g_{m,p,q}}_{q=1}^{Q}. Particularly, for the case of Z people located in the radar's FOV, it is assumed that the frequency set {g_{m,p,q}}_{q=1}^{Q} includes their HR and RR, denoted by fₕ^{(z)} and fᵣ^{(z)}, respectively, for z=1, . . . , Z. Finally, in this model the inventors consider a total of P possible azimuth angles {θₚ}ₚ₌₁^{P}, reflected in the following phase shifts:

$${ϕ}_{p} [ k ] = \frac{2 π}{λ} {r}_{k} sin {θ}_{p} , k = 1 , … , K \leq P . \qquad ( 35 )$$

The slow-time varying complex amplitude is defined as:

$${\tilde{x}}_{m , p} [ l ] {=}^{△} {x}_{m , p} {e}^{j {ψ}_{m , p} [ l ]} , l = 1 , … , L . \qquad ( 36 )$$

For each frame l the samples of y[n, k, l] (1) can be assembled into the matrix [symbol] ₁∈ [symbol] ^{N×K}, which satisfies:

$${𝕐}_{l} = A {𝕏}_{l}^{T} {B}^{T} + {𝕎}_{l} , l = 1 , … , L \qquad ( 37 )$$

where A∈ [symbol] ^{N×M}, M≤N, [symbol] ₗ∈ [symbol] ^{P×M}, B∈ [symbol] ^{K×P}, K≤P and [symbol] ₗ∈ [symbol] ^{N×K}, whose entries are respectively given by:
Ā(n, m)≙e^{j2πf}^{ₘ}^{nT}^{_{f}}, [symbol] ₗ(p, m)≙\tilde{x}_{m,p}[l] of Eq. (6), B(k, p)≙e^{jϕ}^{ₚ}^{[k]} and
[symbol] ₗ(n, k)≙w[n, k, l].

One can verify that for K=1, the model in Eq. (37) coincides with the SISO model described above, Eq. (14).

To facilitate the analysis, in the following the model in Eq. (37) is converted to a single matrix representation for L given frames. To that end, it is first assumed that the fast-time frequencies {fₘ}ₘ₌₁^{M} (Eq. (32)) lie on the Nyquist grid, i.e.,

$${f}_{m} = \frac{{f}_{ADC}}{N} {i}_{m} , {i}_{m} = 0 , … , M - 1 , \qquad ( 38 )$$

where f_{ADC}≙1/T_{f} is determined by the ADC component. Then, by the structure of A in Eq. (37) given Eq. (38), it is possible to construct

$${\overline{𝕐}}_{l} = B {𝕏}_{l} + {\overline{𝕎}}_{l} , l = 1 , … , L$$

where [symbol] ₗ≙1/N(A^{H} [symbol] ₗ)^{T}∈ [symbol] ^{K×M} and

$${\overline{𝕎}}_{l} {=}^{△} \frac{1}{N} {( {A}^{H} {W}_{l} )}^{T} ∈ {ℂ}^{K \times M} .$$

Here, the fact that A^{H}A=NI_{M}, where I_{M} denotes a M-size identity matrix is used, since by Eq. (38),

$$A ( n , m ) = {e}^{j 2 π \frac{im}{N} n} .$$

It is noted that since [symbol] ₗ includes independent and identically distributed Gaussian random variables, the statistical properties of the model in Eq. (37) are preserved. Next, all M columns of [symbol] ₗ are concatenated, to obtain a vectorized representation ŷₗ∈ [symbol] ^{KM×1}

$${\hat{y}}_{l} = C {\tilde{x}}_{l} + {\hat{w}}_{l} , l = 1 , … , L , \qquad ( 39 )$$

where C∈ [symbol] ^{KM×PM} denotes a block matrix containing M blocks of B on its main diagonal, ŵₗ∈ [symbol] ^{KM×1} is the transformed noise vector and based on Eq. (36) and the entries of \tilde{x}ₗ∈ [symbol] ^{PM×1} are given by

$${\tilde{x}}_{l} ( {i}_{m , p} ) = {x}_{{i}_{m , p}} {e}^{j {ψ}_{{i}_{m , p}} [ l ]} , l = 1 , … , L , \qquad ( 40 )$$

with the row index i_{m,p}=(m−1)P+p.

In order to perform continuous NCVSM, the radar is to operate and generate data frames throughout the entire monitoring duration. To this end, at each predefined time interval Tᵢₙₜ, the sequence {ŷₗ}ₗ₌₁^{L} (Eq. (39)) is formed by collecting the last L frames up to that time. The number of frames to be processed, L, is determined by a predefined time window T_{win} according to L=T_{win} fₛ, where the units of T_{win} and fₛ are [s] and [1/s], respectively. Finally, the observations in Eq. (39) are reformulated for L given frames, as follows

$$Y = C \tilde{X} + W , \qquad ( 41 )$$

where Y≙[ŷ₁, . . . , ŷ_{L}]∈ [symbol] ^{KM×L}, \tilde{X}≙[\tilde{x}₁, . . . , \tilde{x}_{L}]∈ [symbol] ^{PM×L} and W≙[w₁, . . . , W_{L}]∈ [symbol] ^{KM×L} is the noise matrix. It is assumed here that only Z<<PM stationary humans are being monitored. Using the above settings, this assumption induces a row-wise sparsity in \tilde{X}, meaning that the vectors {\tilde{x}ₗ}ₗ₌₁^{L} share a joint support.

Based on the model in Eq. (41), the first goal is to recover the row-coordinates of \tilde{X} associated with humans in the radar's FOV, denoted by the support [symbol] , by which the spatial location of each human can be estimated. The second goal, using the recovered [symbol] , is to continuously evaluate the RR and HR of each detected human throughout the complete monitoring duration. Mathematically, every Tᵢₙₜ the inventors seek to estimate the corresponding {fᵣ^{(z)}, fₕ^{(z)}}_{z=1}^{Z} see Eq. (34).

As described above (refer to FIG. 3) the sparsity-based solution according to method of the present disclosure is divided into two main stages after a preliminary processing. In the first stage (step 310), the support [symbol] is recovered, by which the spatial location of each human is estimated (step 312). In the second stage, the simultaneous NCVSM of the detected humans using [symbol] and the described above VSDR method is used, for the discussed scenario (steps 316-318). These stages will be described in detail in the following.

In the preliminary pre-processing (step 304) at each Tᵢₙₜ, the matrix Y of Eq. (41) is assembled from the given radar data samples in Eq. (31) by following relations of Eqs. (37)-(41). This requires first to set the dictionary matrices A and B of Eq. (37). For the matrix A, the frequencies {fₘ}ₘ₌₁^{M} satisfy Eq. (38). As for B, the phase shift ϕₚ[k] (Eq. (35)) is set according to the ULA defined above by {rₖ}ₖ₌₁^{K} and angle grid covering FOV of 180 degrees

$${θ}_{p} = - 90 + {i}_{p} {Δ}_{θ} , {i}_{p} = 0 , … , P - 1 , P = \frac{180}{{Δ}_{θ}} , \qquad ( 42 )$$

where Δ_{θ} denotes the spacing of the angle grid. Thus, by Eqs. (35) and (42), B(k, p)=e^{jπ(k-1)sin(-90+i}^{ₚ}^{Δ}^{_{θ}}⁾.

In the next stage, the multiple people localization is performed. In the first monitoring iteration, the inventors use the assembled matrix Y (41) to estimate the spatial locations of all stationary humans by recovering \tilde{X} and its support [symbol] . This allows to efficiently extract the relevant Doppler samples, from which each individual's vital signs are monitored, for the remainder of the monitoring process.

To aid in the separation of humans from static or vibrating clutter, before recovering \tilde{X}, spectral filtering of Y is performed in the slow-time axis based on prior knowledge of human-typical pulse and breathing rates. To that end, the frequency bands of normal respiration and heartbeat are denoted by B^{(R)} and B^{(H)}, respectively. The filtered signal is then given by

$$\overline{Y} = \frac{1}{L} {( {F}_{L}^{H} ( Π ⊙ {F}_{L} {Y}^{T} ) )}^{T} , \qquad ( 43 )$$

where F_{L} is a full L-size DFT matrix, Π denotes an ideal window corresponding to the vital frequencies in B^{(H)}∪B^{(R)} and ⊙ denotes the element-wise product.

Since it is assumed that \tilde{X} is a row-sparse matrix, this matrix is recovered from Y̅ using C and a JSR technique [18], formulated by the following optimization problem:

$${min}_{\hat{X} ∈ {ℂ}^{PM \times L}} {‖ \overline{Y} - C \tilde{X} ‖}_{F}^{2} + γ {‖ \tilde{X} ‖}_{2 , 1} \qquad ( 44 )$$

Here, to promote the row sparsity of \tilde{X}, inspired by reference [18], the inventors use the regularization parameter γ>0 and the mixed l_{2,1} norm defined by ∥X∥_{2,1}≙Σᵢ∥xⁱ∥₂, with xⁱ denoting the i'th row of a matrix X. Similarly to [18], Eq. (44) is solved using the fast iterative soft-thresholding algorithm (FISTA).

Now, the support [symbol] can be obtained by selecting the most dominant rows of the recovered \tilde{X} according to the average power [11] of each row. It should be noted that according to Eq. (40) and since \tilde{X}≙[\tilde{x}₁, . . . , \tilde{x}_{L}], each row index of \tilde{X} satisfies i_{m,p}=(m−1)P+p. Hence, by using the modulo operation on the indexes i_{m,p}∈ [symbol] , the inventors estimate both the distance dₘ and angle θₚ of each monitored individual with respect to the radar, through Eq. (32) and the grids in Eqs. (38) and (42). It should be noted that since the humans are stationary, the coordinates of support S are fixed throughout the monitoring, implying that it is required to recover [symbol] only once, and the same [symbol] (i.e., the same row-index set) can be used for all subsequent iterations.

Since \tilde{X} is a row-sparse matrix based on support [symbol] , the model in (41) can be written as

$$Y = {C}_{s} {\tilde{X}}_{s} + W \qquad ( 45 )$$

with [symbol] ∈ [symbol] ^{KM×Z} and [symbol] ∈ [symbol] ^{Z×L} respectively being the atoms of C and the rows of \tilde{X} corresponding to [symbol] , where Z=| [symbol] |. By holding the support, for each Tᵢₙₜ the inventors can directly estimate \tilde{X} [symbol] from Y, using the Least-Squares (LS) solution given by

$${\overline{x}}_{s} = {( {C}_{s}^{H} {C}_{S} )}^{- 1} {C}_{s}^{H} Y \qquad ( 46 )$$

considering that Z<<KM.

Using Eq. (40) and the definition of \tilde{X} below (Eq. (41)), it can be shown that

$${\tilde{X}}_{S} ( {i}_{m , p} , l ) = {x}_{{i}_{m , p}} {e}^{j {ψ}_{{i}_{m , p}} [ l ]} , {i}_{m , p} ∈ S .$$

That is [symbol] estimates the slow-time varying phasor terms associated with humans in the radar's FOV that contain their vital information in the phase terms ψᵢ_{_{m,p}}[l], i_{m,p}∈ [symbol] (Eq. (33)). Hence, similarly to the procedure described above, the inventors use the four-quadrant arctangent function on each element of [symbol] followed by unwrapping based on technique of reference [11], to yield the vibration matrix V∈ [symbol] ^{L×Z}. The matrix V can be viewed as a chain of vectors corresponding to the thoracic vibration pattern of each detected human, i.e., V=[v₁, . . . , v_{Z}] where each v_{z}∈ [symbol] ^{L} contains a scaled approximation of the samples {vᵢ_{_{m,p}}[l]}ₗ₌₁^{L} (Eq. (34)) for i_{m,p}∈ [symbol] .

In the final stage of each iteration, both the RR and HR of each individual, {fᵣ^{(z)}, fₕ^{(z)}}_{z=1}^{Z}, are estimated simultaneously given V, B^{(R)} and B^{(H)}, and recorded for continuous NCVSM. Here, the inventors use the VSDR method described in detail above, which exploits appropriate dictionaries to search for the desired rates over high-resolution grids corresponding to human cardiopulmonary activity.

In the following, the performance of the proposed approach is evaluated and compared to existing techniques, through a simulation based on the model in Eq. (31) that involves real Electrocardiography (ECG) impedance data from [19], which is divided into two parts. The first investigates spatial localization of multiple people in a cluttered environment and the second examines NCVSM given the extracted thoracic vibration of each detected human, versus SNR.

To this end, the inventors generated 5 different objects (of which Z=3 humans) in the radar's FOV using the proposed data model in Eq. (31). Each object is characterized by a different set of x_{m,p} (1), dₘ (2), θp (5) and {v_{m,p}[l]}ₗ₌₁^{L} (Eq. (34)), as detailed in Table 5.

It should be noted that at a distance of dₘ=0.98 [m] from the radar there are 2 humans and a vibrating fan (with a_{fan}=10⁻² and f_{fan}=20 [Hz]), whose positions differs only in their azimuth angle. To relate to real thoracic vibrations, the inventors used the 10-minute long 100 [Hz] impedance cardiography signals of subjects 4-6 from [19]'s resting scenario (in which participants were told to breath calmly and avoid large movements), for Table 5's vibrations {v_{m,p}[l]}ᵢ₌₁^{L}, with proper adjustments.

Since a variety of respiratory parameters can be extracted from the impedance signal, including RR, the raw signal serves as a reference for comparing the RR estimates. As to the HR reference, the inventors used the gold-standard 2000 [Hz] lead-2 ECG signal from [19], and down-sampled it to 100 [Hz] to correspond to Tₛ=10 [ms]. The rest of the parameters for assembling the model in Eq. (31) were set as follows: λ=3.9 [mm], T_{c}=57 [μs], f_{ADC}=4 [MHz], S=70 [MHz/μs], M=N/2=100 and K=4. Furthermore, to examine the impact of environmental noise, the inventors used an SNR term that controls the variance of {w[n, k, l]} in Eq. (31) via SNR≙1/σ². Finally, the frequency bands of respiration and heartbeat were set to B^{(R)}=[0.1 0.4][Hz] and B^{(H)}=[0.78 1.67][Hz], respectively, corresponding to a normal resting state.

Using all the above specifications, the inventors simulated a localization and 10-minute NCVSM of 3 people simultaneously, with RR and HR estimates computed every Tᵢₙₜ=0.05 [s], using L data frames from the last T_{win}=30 [s], starting at T_{win}.

TABLE 5

| Object type | x_{m,p} | dₘ[m] | θₚ[°] | {v_{m,p}[l]ₗ₌₁^{L} |
|---|---|---|---|---|
| Static clutter | 1 | 0.5 | 0 | 0 |
| Human #1 | 0.3 | 0.98 | −30 | Based on [19] |
| Vibrating fan | 0.3 | 0.98 | 0 | α_{fan} cos(2πf_{fan} lTₛ) |
| Human #2 | 0.3 | 0.98 | +30 | Based on [19] |
| Human #3 | 0.2 | 1.5 | +15 | Based on [19] |

In the following, the inventors compared the proposed JSR localization method to methods based on the Angle-FFT [7] and MUSIC-DOA [22,23] approaches, for localizing the considered humans.

The parameters of the proposed JSR were set as follows. The frequencies of Π in Eq. (43) were drawn from the length-L Nyquist grid determined by fₛ, the parameters for solving Eq. (44) using FISTA] were set to γ=5, the Lipschitz constant to 4.5e6 and 1000 iterations were considered. Finally, the angle grid spacing was set to Δ_{θ}=1. For a fair comparison to the other techniques, all are based on the same angular grid.

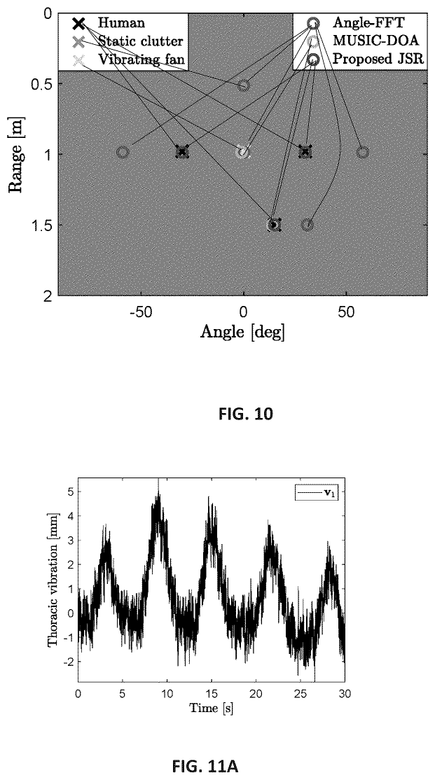

FIG. 10 shows the spatial estimates by the examined techniques for SNR=0 [dB]. One can observe that only the proposed JSR method indicates the correct locations (both distance and angle) of the humans compared to the intensity-based Angle-FFT method [7] and the MUSIC-DOA approach [22, 23]. Note that the Angle-FFT method suffers from gross errors especially for dₘ=0.98 [m] where there are multiple equidistant objects, since the theoretical angular resolution is ≈30° for K=4 receivers. The MUSIC-DOA approach seeks for highly oscillating objects and thus for dₘ=0.98 it incorrectly selects the vibrating fan over the humans. In contrast to the compared localization techniques, the proposed JSR exploits both characteristics of human-typical vital frequencies and prior assumption on the sparse structure of \tilde{X}.

Given a successful human localization from the previous study, the inventors examined the performance of VSDR for NCVSM, and compared it to other state-of-the-art techniques based on [8-12, 15].

To compare fairly (regardless of localization performance), the inventors examined only the last step of the algorithm of the present disclosure, which estimates human vital signs given the extracted matrix V. In addition, all methods used the same frequency bands B^{(R)} and B^{(H)}, and the same stability enhancement procedure by replacing the RR and HR estimates with the average of the last 0.7 seconds' estimates.

The VSDR approach was first compared to the method detailed in [8] for estimating RR and HR given the phase of an FMCW signal, called here Phase-Reg. Moreover, VSDR was compared to an FFT-based peak selection in each frequency band, with zero-padding (FFT w/ZP) [15] and without (FFT w/o ZP) [8-12]. The padding of FFT w/ZP was set to fit a 60-second time window corresponding to frequency resolution of 1 [bpm]. The RR and HR of the reference data was estimated via the DFT spectrum, similarly to [8-11], with padding to fit a 60-second time window for increased resolution, presuming they are noise-free.

The same performance metrics were used here, as already described in relation to the single-receiver method. Since NCVSM of Z=3 people are investigated for different SNR cases, the performance score produced for each metric and SNR is regarded as the average across the participants' scores. It is noted that the simulation settings bring to 11400 non-contact HR/RR estimates for each participant to compare to the contact HR/RR reference estimates.

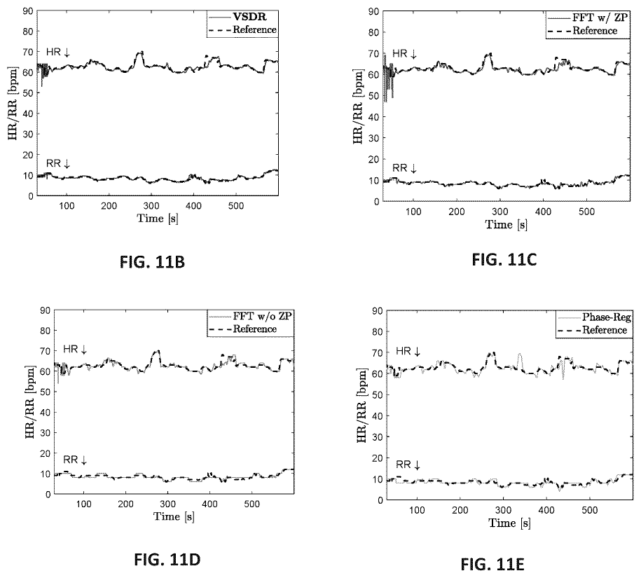

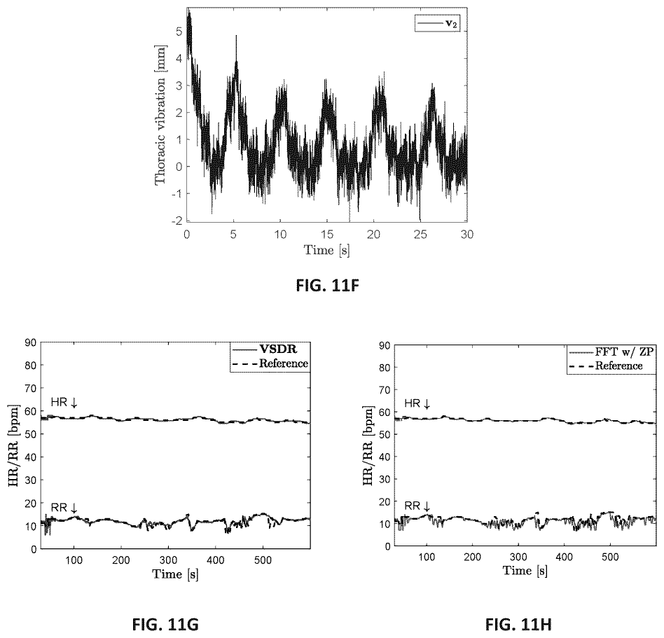

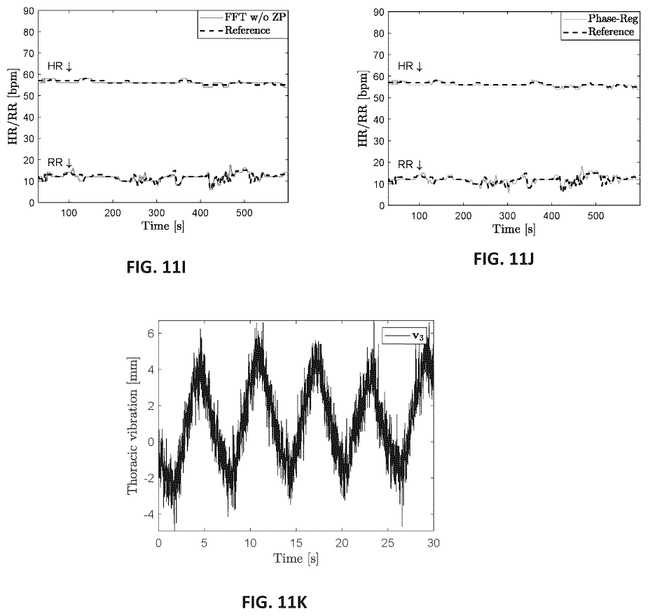

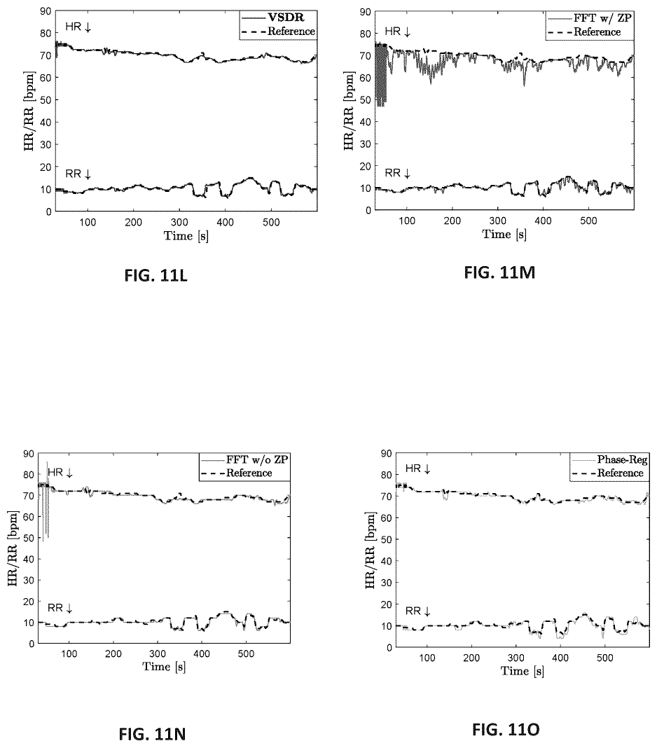

FIGS. 11A to 11O exemplify the performance of NCVSM system compared to the references (ground truth) by VSDR and the other examined techniques, for SNR=0 [dB]. Data are shown for humans listed in Table 5, human #1 (FIGS. 11A to 11E), human #2 (FIGS. 11F to 11J), and human #3 (FIGS. 11K to 11O) respectively; wherein

FIGS. 11A, 11F, and 11K show the respective vectors v of thoracic vibration patterns of each detected human; for each detected human, respectively: FIGS. 11B, 11G, and 11L show estimated vital signs obtained by the VSDR approach; FIGS. 11C, 11H, and 11M show estimated vital signs obtained by the FFT-based peak selection with zero-padding (ZP) approach; FIGS. 11D, 11I, and 11N show estimated vital signs obtained by the FFT-based peak selection without ZP approach; and FIGS. 11E, 11J, and 11O show estimated vital signs obtained by Phase-Reg approach detailed in [8], based on the phase of an FMCW signal;

It can be seen how both the HR and RR estimated by the VSDR approach show great resemblance to those of the reference, compared to the others in which the noisy setup impairs their assessments.

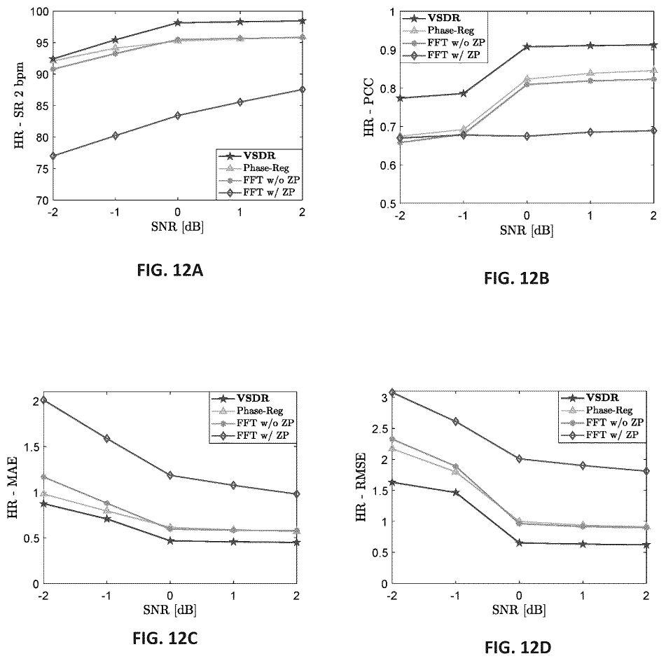

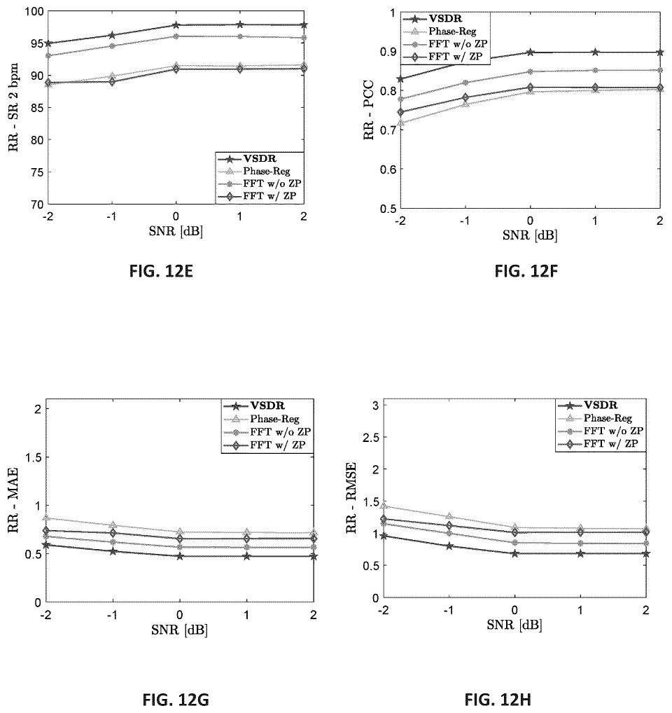

FIGS. 12A to 12H illustrate comparison between HR and RR monitoring performance using the techniques of FIGS. 11A to 11O as a function of
SNR (SNR∈[−2,2]) according to several statistical metrics, wherein FIG. 12A shows HR Success Rate; FIG. 12B shows HR PCC (Pearson Correlation Coefficient); FIG. 12C shows HR MAE (Mean-Absolute Error); FIG. 12D shows HR RMSE (Root-Mean-Square Error); FIG. 12E shows RR Success Rate; FIG. 12F shows RR PCC; FIG. 12G shows
RR MAE and FIG. 12H shows RR RMSE;

This comparison demonstrates that VSDR outperforms the other compared methods in all 4 metrics, for every SNR value.

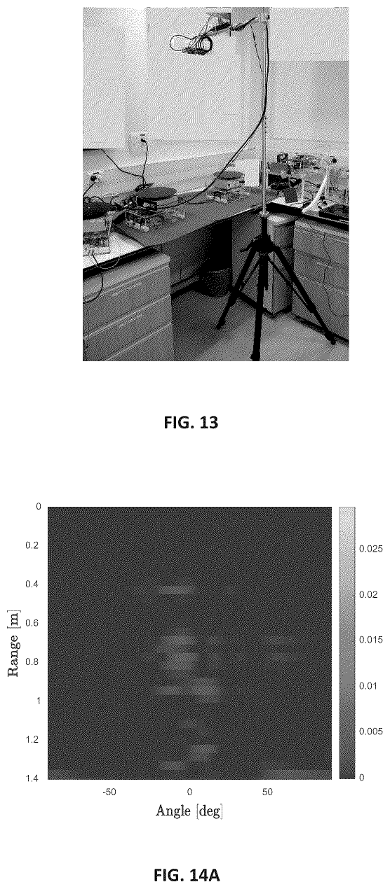

The inventors further used an experimental setup simulating a multi-person localization and NCVSM. This is illustrated in FIG. 13: two vibrating plates, imitating two humans, were placed at radial distance 0.95 [m] from the radar and at azimuth angles +−13 [deg], each plate vibrating according to different real human breathing pattern.

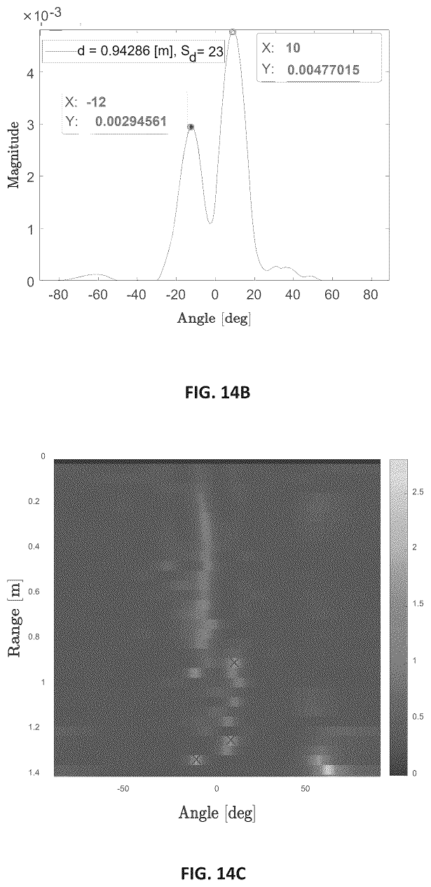

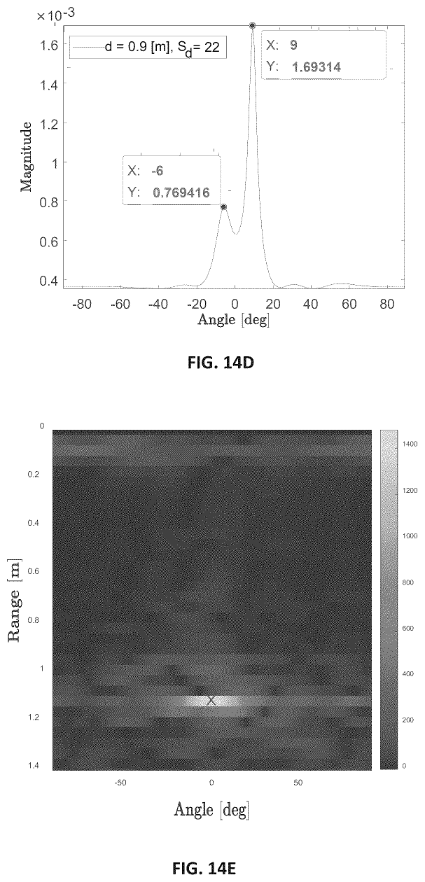

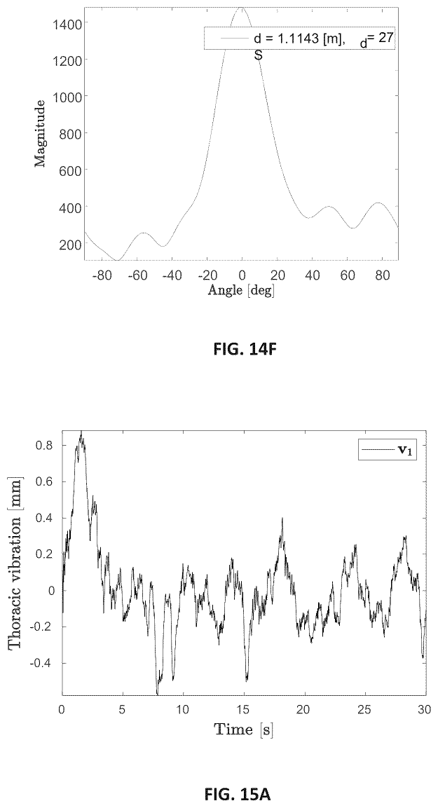

FIGS. 14A to 14F show the results of multiple people localization utilizing the setup of FIG. 13 using the JSR (Joint Sparse Recovery) technique of the present disclosure compared to known-in-the-art approaches: MUSIC-DOA approach [22-23] and intensity-based Angle-FFT method [7], wherein FIGS. 14A, 14C, 14E show the localization maps (Range vs. Angle) with JSR, MUSIC-DOA and Angle-FFT methods, respectively; and FIGS. 14B, 14D, 14F show the respective localization curves (Magnitude at localized “person” vs. Azimuthal angle) obtained from the maps of FIGS. 14A, 14C, 14E. The results clearly show that the JSR (Joint Sparse Recovery) technique of the present disclosure successfully localizes the radial distances and the azimuthal angles of both humans, whereas the other known-in-the-art approaches either obtain wrong localization coordinates or altogether fail to localize the two humans.

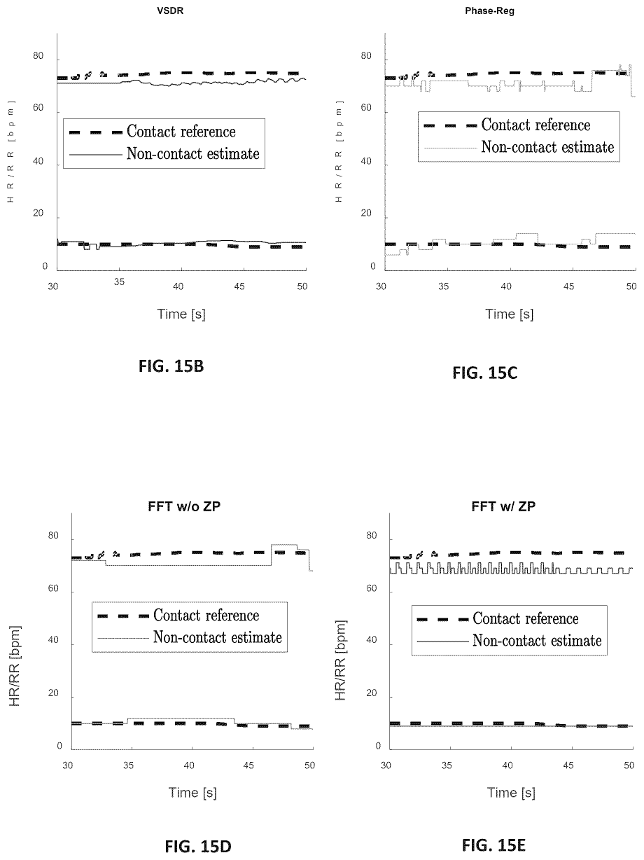

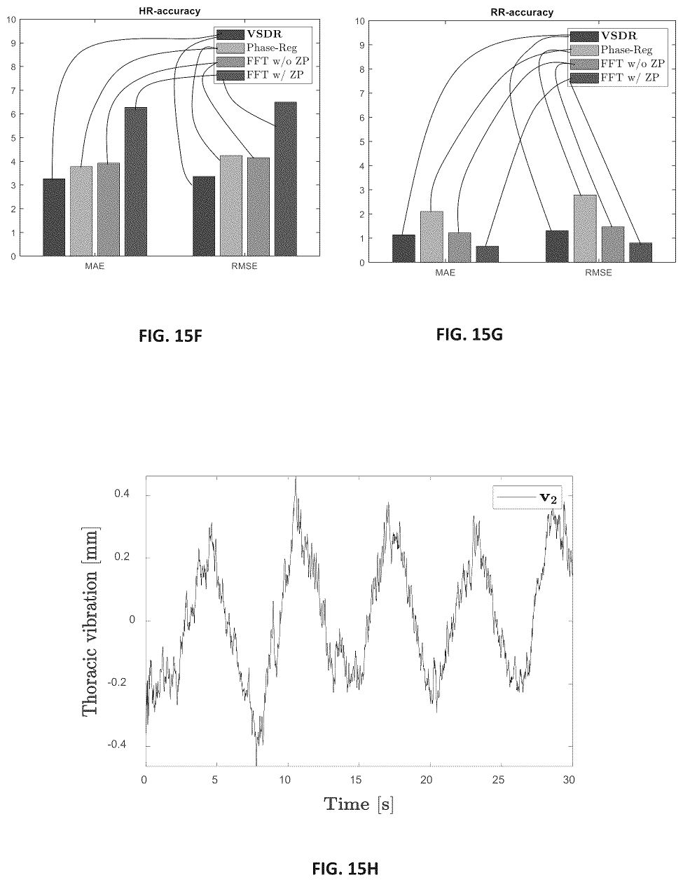

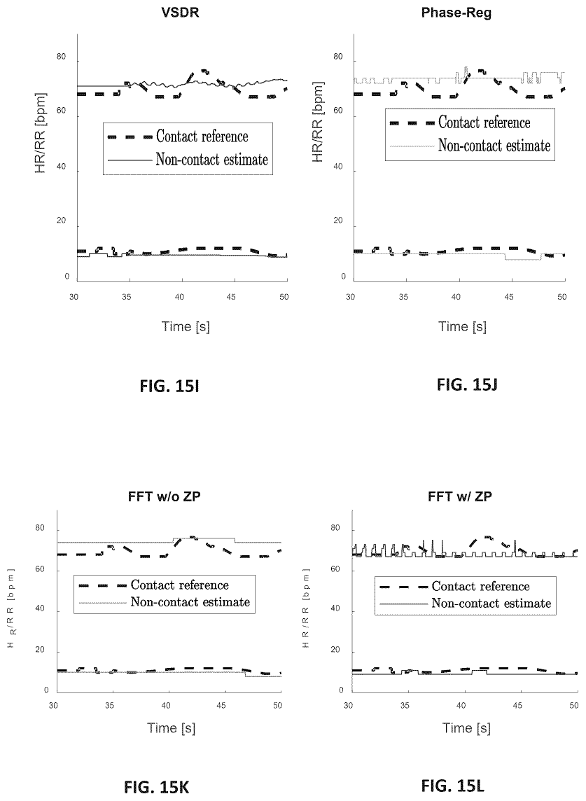

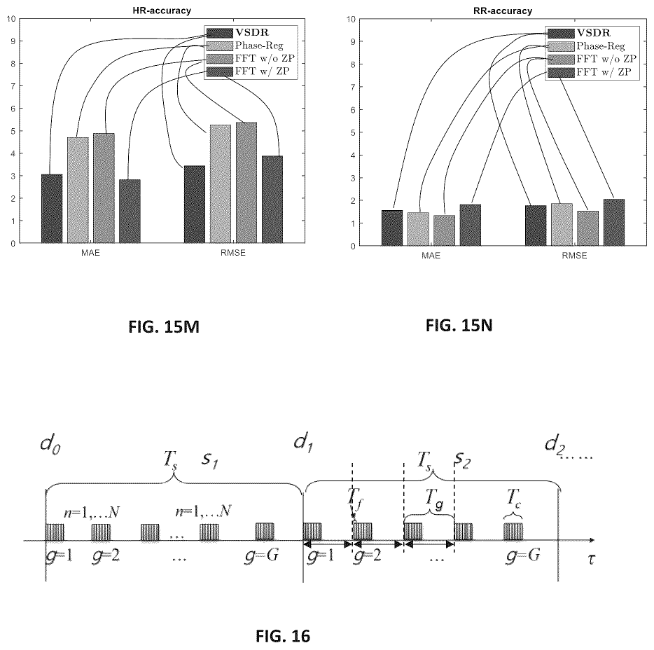

FIGS. 15A to 15N exemplify the performance of NCVSM system compared to the references (ground truth) by VSDR and the other examined techniques, Data are shown for humans shown in the experimental setup of FIG. 13. The results for human #1 and human #2 are shown in FIGS. 15A to 15G and FIGS. 15H to 15N, respectively. FIGS. 15A and 15H, show the respective vectors v₁ and v₂ of thoracic vibration patterns of each detected human. For each detected human, respectively: FIGS. 15B and 15I, show estimated vital signs obtained by the VSDR approach; FIGS. 15C and 15J show estimated vital signs obtained by Phase-Reg approach detailed in [8], based on the phase of an FMCW signal; FIGS. 15D and 15K show estimated vital signs obtained by the FFT-based peak selection without ZP approach; and FIGS. 15E and 15L show estimated vital signs by the FFT-based peak selection with zero-padding (ZP) approach. FIGS. 15F, 15G, 15M and 15N illustrate comparison between HR and RR accuracy using the techniques of FIGS. 15B to 15E and FIGS. 15I to 15L according to several statistical metrics, wherein FIGS. 15F and 15M show HR MAE (Mean-Absolute Error) and RMSE (Root-Mean-Square Error), and FIGS. 15G and 15N show RR MAE (Mean-Absolute Error) and RMSE (Root-Mean-Square Error).

In this disclosure, a novel extended FMCW signal models for NCVSM of multiple people was described, allowing to interpret a realistic noisy environment containing multiple objects. Based on this model, the inventors utilized a JSR approach that accurately localizes multiple people in a clutter-rich scenario, based on the sparse composition of the input data, where the known localization methods perform poorly. The inventors also developed the VSDR method, which performs accurate NCVSM given human thoracic vibrations, by leveraging human-typical cardiopulmonary characteristics using a dictionary-based approach. The robustness of proposed VSDR is reflected in superior performance results using data from 30 monitored individuals, outperforming state-of-the-art alternative techniques using multiple statistical criteria.

The above-described examples of the technique of the present disclosure relate to the use of SISO FMCW radar system for vital signs monitoring of static subjects. In the following, a model for NCVSM of multiple moving people based on SISO FMCW radar is described. The inventors present a sparsity-based approach using this model that can accurately localize targets during moving. The heartbeat signals are extracted from the mixed phase by solving a signal decomposition problem. This method is validated through synthetic data of two moving people yielding improved performance when compared to other existing techniques.

Let us consider the FMCW radar successively transmitting a saw-tooth chirp at each time frame to M stationary targets in the radar's field of view (FOV). According to the model described above with respect to static subjects/humans (refer to Eq. (1) above), the distance between the radar and a target m, given m=1, . . . , M, is defined as a combination of an initial distance dₘ⁰ and chest vibration distance dₘ^{v}[l′] modeled as a sum of cosines, i.e.,

$${d}_{m} [ {l}^{′} ] {=}^{△} {d}_{m}^{0} + {d}_{m}^{v} [ {l}^{′} ] , \qquad ( 47 )$$

with dᵥ^{m}[l′]≙Σ_{q=1}^{Q} a_{m,q} cos(2πf_{m,q}^{v}l′T_{g}), where l′=1, . . . , L′ is the frame index, T_{g} is the frame duration, also known as slow time index, with L′ being the total number of frames. a_{m,q} and f_{m,q}^{v} denote the amplitudes and frequency of vibrations, including those related to vital signs. Suppose each chirp is sampled to a length N discrete signal with sampling interval T_{f}, the FMCW 2D beat signal for M static targets is represented by

$$y [ n , {l}^{′} ] {=}^{△} {\sum }_{m = 1}^{\overline{P}} {x}_{m} exp ( j ( 2 π {f}_{m} n {T}_{f} + {ψ}_{m} [ {l}^{′} ] ) ) + w [ n , {l}^{′} ] \qquad ( 48 )$$

with n=1, . . . , N, l′=1, . . . , L′, where c is speed of light, S is the chirp frequency sweeping rate, λₘₐₓ is the maximal wavelength of the chirp, xₘ is the amplitude of the target m, and w is the noise. {fₘ}ₘ₌₁^{M} denotes all possible frequencies related to the target range distances

$${\{ {d}_{m}^{0} \}}_{m = 1}^{M} . {ψ}_{m} [ {l}^{′} ] {=}^{△} \frac{4 π}{{λ}_{max}} {d}_{m} [ {l}^{′} ] = \frac{4 π}{{λ}_{max}} ( {d}_{m}^{0} + {d}_{m}^{v} [ {l}^{′} ] )$$

is the slow time phase that is only changed by small vibrations dₘ^{v}[l′].

In the following, the inventors consider monitoring targets with large movements. First a new time scale, window, is defined for the model. Given a monitoring segment of frames, they are divided into L windows, each containing G frames, as in FIG. 16. The duration of a window is Tₛ, with Tₛ≙GT_{g}. The index of windows and frames in a window are denoted by l and g, respectively, with l=1, . . . , L and g=1, . . . , G. Consequently, the global index of frames, l′, can be computed by l′=(l−1)G+g. The inventors adopt a window rate at least several times higher than the maximal Nyquist frequency of heartbeat and motion such that the velocity of both can be considered constant inside each window. This division enables the inventors to flexibly describe movement distance with velocity and leverage joint sparsity within a window for accurate localization, as demonstrated below. In the following, the 2-D index, [l, g], is used to replace the [l′].

It is noted that here G is used to denote the number of frames within a window, whereas in the description above, related to static subjects, G was used to denote the number of chirps per frame.

For moving targets, the time-varying distance sampled at the g'th frame of window I between the radar and the m-th target can be represented as a combination of initial distance dₘ⁰, movement distance dₘ^{w}, and vibration distance dₘ^{v}:

$${d}_{m} [ l , g ] {=}^{△} {d}_{m}^{0} + {d}_{m}^{w} [ l , g ] + {d}_{m}^{v} [ l , g ] , \qquad ( 49 )$$

with l=1, . . . , L and g=1, . . . , L. Next, the velocity is used to describe different parts of the distance.

To mimic the slow moving pattern of humans w.r.t windows, the moving velocity of each human is modeled as a piecewise constant function of l. Within L windows, there are P<L segments of different velocities, whose duration time can be different. The velocity of target m during the p-th segment [l_{m,p-1}, l_{m,p}) is denoted as z_{m,p}, with p=1, . . . , P, l_{m,p-1} and l_{m,p} being the starting and ending index of the segment. Defining an indicator function 1_{[l}_{_{m,p−1}}_{,l}_{_{m,p}}₎[l], such that 1_{[l}_{_{m,p−1}}_{,l}_{_{m,p}}₎[l] is equal to 1 if l∈[l_{m,p-1}, l_{m,p}) and 0 otherwise, the moving velocity sₘ^{w}[l] can be represented as

$${s}_{m}^{w} [ l ] {=}^{△} {\sum }_{p = 1}^{P} {z}_{m , p} {1}_{[ {l}_{m , p - 1} , {l}_{m , p} )} [ l ] , l = 1 , … , L \qquad ( 50 )$$

Referring to FIG. 16, dₘ[l, g] can be described as a cumulative summation of segment distance computed from movement velocity, i.e., dₘ^{w}[l, g]≙Σᵢ₌₁^{l-1} sₘ^{w}[i]Tₛ+sₘ^{w}[l]gT_{g}, with l=1, . . . , L, g=1, . . . , G. Similarly, dₘ^{v}[l, g] can also be computed from the discrete vibration velocity, sₘ^{v}[l], l=1, . . . , L, by dₘ^{v}[l, g]=Σᵢ₌₁^{l-1} sₘ^{v}[i]Tₛ+sₘ^{v}[l]gT_{g}, with l=1, . . . , L, g=1, . . . , G, and

$${s}_{m}^{v} [ l ] {=}^{△} {\sum }_{q = I}^{Q} {a}_{m , q}^{′} sin ( j 2 π {f}_{m , q}^{v} l {T}_{s} ) \qquad ( 51 )$$

where a′_{m,q} denotes the amplitudes of the q-component of velocity of target m.
Define total velocity as

$${s}_{m} [ l ] {=}^{△} {s}_{m}^{w} [ l ] + {s}_{m}^{v} [ l ] , \qquad ( 52 )$$

with l=1, . . . , L. Using the expression of dₘ^{w}[l] and dₘ^{v}[l], Eq. (49) becomes

$${d}_{m} [ l , g ] = {d}_{m}^{0} + {\sum }_{i = 1}^{l - 1} {s}_{m} [ i ] {T}_{s} + {s}_{m} [ l ] g {T}_{g} = {d}_{m}^{0} + {\sum }_{i = 1}^{l - 1} ( {s}_{m}^{w} [ i ] + {s}_{m}^{v} [ i ] ) {T}_{s} + ( {s}_{m}^{w} [ l ] + {s}_{m}^{v} [ l ] ) g {T}_{g} , \qquad ( 53 )$$

with l=1, . . . , L and g=1, . . . , G.

Based on Eq. (48), the 2-D beat signal for M moving objects at frame g in window l is represented by substituting dₘ[l′] of Eq. (48) with Eq. (53), resulting in

$${y}_{l} [ n , g ] {=}^{△} {\sum }_{m = 1}^{M} {x}_{l , m} exp ( j ( 2 π {f}_{m} n {T}_{f} + {ψ}_{l , m}^{s} [ g ] ) ) + {w}_{l} [ n , g ] , \\ with \qquad ( 54 )$$

$${ψ}_{l , m}^{s} [ g ] {=}^{△} \frac{4 π}{{λ}_{max}} {d}_{m} [ l , g ] = \frac{4 π}{{λ}_{max}} ( {d}_{m}^{0} + {\sum }_{i = 1}^{l - 1} {s}_{m} [ i ] {T}_{s} + {s}_{m} [ l ] g {T}_{g} ) , \qquad ( 55 )$$

where x_{l,m} is the amplitude of target m in window l, which is zero if the target is not detected at the fₘ. The fast-time frequency fₘ can be regarded as constant in window l, since fₘ is insensitive to small displacement, considering that Tₛ is relatively short w.r.t body movement. The slow-time varying phase ψ_{l,m}^{s}[g] (Eq. (55)), in contrast, is sensitive to small movements, meaning that the distance change between frames inside a window should be considered. It is noted that when G=1 and {sₘ^{w}[i]}ᵢ₌₁^{L}=0, Eqs. (54) and (55) reduce to the model of Eq. (48) for a single l in the static case described above, based on Eq. (51).

$$Define {\tilde{x}}_{l , m}^{s} [ g ] {=}^{△} {x}_{l , m} exp ( j {ψ}_{l , m}^{s} [ g ] ) , \\ \text{Then, Eq . (54) becomes} \\ {y}_{l} [ n , g ] = {\sum }_{m = 1}^{M} {\tilde{x}}_{l , m}^{s} [ g ] exp ( j2 π {f}_{m} n {T}_{f} ) + {w}_{l} [ n , g ] \qquad ( 56 )$$

For each g=1, . . . , G, the fast time samples of yₗ[n, g] are assembled into a vector, resulting in

$${y}_{l , g} = A {\tilde{x}}_{l , g}^{s} + {w}_{l , g} , \qquad ( 57 )$$

where y_{l,g}≙[yₗ[1, g], . . . , yₗ[N, g]]^{T}∈ [symbol] ^{N}, A∈ [symbol] ^{N×M}
with A(n, m)≙exp(j2πfₘnTₛ), \tilde{x}_{l,g}^{s}≙[\tilde{x}_{l,1}^{s}[g] . . . , \tilde{x}_{l,m}^{s}[g]]^{T}∈ [symbol] ^{M},
and w_{l,g}≙[wₗ[1, g], . . . , wₗ[N, g]]^{T}∈C^{N} is the noise vector.

For all frames g=1, . . . , G in window l, Eq. (57) is reformulated in a matrix form:

$${Y}_{l} = A {X}_{l}^{s} + {W}_{l} , l = 1 , … , L \qquad ( 58 )$$

where Yₗ≙[y_{l,1}, . . . , y_{l,G}]∈ [symbol] ^{N×G} is the beat signal matrix, Xₗ^{s}≙[\tilde{x}_{l,1}^{s}, . . . , \tilde{x}_{l,G}^{s}]∈ [symbol] ^{M×G}, and Wₗ≙[w₁^{l}, . . . , w_{l,G}]∈ [symbol] ^{N×G} is a noise matrix.

There are two main differences between Eq. (58) and the static model described above. First, in the static model, the location of nonzero rows in {Xₗ^{s}}ₗ₌₁^{L} is fixed, whereas in the model described for the moving humans described here, these locations vary in different windows and are only fixed within a single window l. Second, the phase term of Xₗ^{s} in the static model does not consider body movements and thus the human distance is modeled as a vibration distance dₘ^{v}[l′] added to a constant dₘ⁰. In the present model, the distance {{dₘ[l, g]}_{g=1}^{G}}ₗ₌₁^{L} contains unknown {{dₘ^{w}[l, g]_{g=1}^{G}}ₗ₌₁^{L} and {{dₘ^{v}[l, g]}_{g=1}^{G}}ₗ₌₁^{L} described by {sₘ^{w}[l]}}ₗ₌₁^{L} and {sₘ^{v}[l]}ₗ₌₁^{L}, respectively, which requires additional efforts to separate.

It is noted that Xₗ^{s} is assumed to have the following properties:
(A-1) Only U<<M humans are being monitored, and the body movements within a window are small. This introduces row-wise sparsity in Xₗ^{s} for each window, meaning that the columns of Xₗ^{s} for each l share a joint support.
(A-2) From Eq. (51), {sₘ^{v}[l]}ₗ₌₁^{L} is band-limited, and the heartbeat band B is assumed to be known a priori. This implies that the energy of the heartbeat signal outside band B is approximately zero:

$${\sum }_{k = 0}^{\frac{L - 1}{2}} {\vert {\sum }_{l = I}^{L} {e}^{- i \frac{2 π}{L} l k} {s}_{m}^{v} [ l ] \vert}^{2} \approx 0 , \frac{k}{{T}_{s} L} ∉ B , \qquad ( 59 )$$

supposing that L is odd.
(A-3) There are no sharp breaks or jumps appear in Eq. (51) and its derivative, resulting in smooth time-varying {sₘ^{w}[l]}ₗ₌₁^{L}, i.e., Σₗ₌₁^{L-1} (sₘ^{v}[l]−sₘ^{v}[l−1])² is small.
(A-4) Only P<<L (In Eq. (50)) possibilities of velocity change, leading to a limited number of accelerations, which means that {|sₘ^{w}[l]−sₘ^{w}[l−1]{ₗ₌₁^{L} is sparse.

The goal of the inventors is to recover the velocity of U humans, {sᵤ^{w}[l], sᵤ^{v}[l]}ₗ₌₁^{L}}ᵤ₌₁^{U}, which belong to the set {sₘ^{w}[l], sₘ^{v}[l]}ₗ₌₁^{L}}ₘ₌₁^{M}, from {Yₗ}ₗ₌₁^{L}, and estimate their HR from {sᵤ^{v}[l]}ᵢ₌₁^{L}}ᵤ₌₁^{U}. The solution is divided into two steps. The first step is to recover the row coordinates of Xₗ^{s} associated with U targets in all windows and extract the corresponding phase {{Φ_{l,u}^{s}[g]}_{g=1}^{G}}ₗ₌₁^{L} for all humans. The second goal, after converting {{Φ_{l,u}^{s}[g]}_{g=1}^{G}}ₗ₌₁^{L}, u=1, . . . , U, to {{sᵤ[l]}ₗ₌₁^{L}}ᵤ₌₁^{U}, based on Eq. (55), is to decompose {{sᵤ[l]}ₗ₌₁^{L}}ᵤ₌₁^{U} into {sᵤ^{w}[l]}ₗ₌₁^{L}}ᵤ₌₁^{U} and {sᵤ^{v}[l]}ₗ₌₁^{L}}ᵤ₌₁^{U} by leveraging their properties in time and frequency domain, after which the HR is estimated from {sᵤ^{v}[l]}ₗ₌₁^{L}}ᵤ₌₁^{U} using the VSDR method described above with respect to the static model.

The following exemplifies the sparsity-based localization and phase estimation:

Since it is assumed that Xₗ^{s} is a row sparse matrix, it is recovered from Yₗ for all windows l=1, . . . , L using A and a joint sparsity recovery technique, [18], formulated by

$${min}_{{X}_{ι}^{s} ∈ {ℂ}^{M \times G}} \{ {‖ {Y}_{l} - A {X}_{l}^{s} ‖}_{F}^{2} + γ {‖ {X}_{l}^{s} ‖}_{2 , 1} \} \qquad ( 60 )$$

where ∥Xₗ^{s}∥_{2,1}≙Σᵢ∥xⁱ∥, is the mixed l_{2,1} norm with xⁱ denoting the i-th row of Xₗ^{s}, and γ is regularization parameter. The inventors construct A by setting fₘ to lie on the Nyquist grid

$${f}_{m} = \frac{{f}_{A D C}}{N} m ,$$

similar to the method described above. Eq. (60) is solved using the fast iterative soft-thresholding algorithm (FISTA).

The support in window l, denoted by [symbol] [l], is obtained by selecting the most dominant rows of the recovered Xₗ^{s} according to the average power of each row, similar to the method described above. Using the support and the sparsity assumption, the model in Eq. (58) can be written as

$${Y}_{l} = {A}_{𝒮 [ l ]} {X}_{l , 𝒮 [ l ]}^{s} + {W}_{l} \qquad ( 61 )$$

with A [symbol] _{[l]}∈ [symbol] ^{N×U} and X_{l,} [symbol] _{[l]}^{s}∈ [symbol] ^{U×G} respectively being the atoms of A and the rows of Xₗ^{s} corresponding to [symbol] [l]. By forming a least-squares problem, [symbol] can be solved for each l:

$${\hat{X}}_{l , 𝒮 [ l ]}^{s} = {( {A}_{𝒮 [ l ]}^{H} {A}_{𝒮 [ l ]} )}^{- 1} {A}_{𝒮 [ l ]}^{H} {Y}_{l} = \frac{1}{N} {A}_{𝒮 [ l ]}^{H} {Y}_{l} . \qquad ( 62 )$$

with [symbol] =NI_{U} and I_{U} denoting a U-size identity matrix. After { [symbol] }ₗ₌₁^{L} is obtained, the phase can be extracted as described above with respect to the static model and obtain the estimated phase matrix Ψ=[\hat{ψ}₁, . . . , \hat{ψ}_{U}]∈ [symbol] ^{GL×U}, where \hat{ψ}ᵤ=[{ψ_{1,u}^{s}[g]}_{g=1}^{G}, {ψ_{2,u}^{s}[g]}_{g=1}^{G}, . . . , {ψ_{L,u}^{s}[g]}_{g=1}^{G}]∈ [symbol] ^{GL} is an estimation of the time- varying phase of the u'th human.

Vital signs extraction can be implemented as follows:

Based on Eq. (55), the estimated distance of the u-th person is

$${\hat{d}}_{u} = \frac{{λ}_{max}}{4 π} {\hat{ψ}}_{u}$$

∈ [symbol] ^{GL} due to both moving and vibration. Generally, body movements (such as several centimeters) are much larger than the vibration caused by heartbeats (usually in millimeters), so the latter tends to be overwhelmed in the mixed signal. If the movement distance is known exactly, we may subtract it from the estimated distance to get chest vibrations. However, direct estimation of movement distances is challenging because it is mixed with chest vibrations. The following solution addresses this challenge by solving a signal decomposition problem.

The inventors first take the difference of \hat{d}ᵤ every G frames to estimate velocity. The vector form of Eq. (52) is written as sₘ=sₘ^{w}+sₘ^{v}∈ [symbol] ^{L}. The elements of ŝᵤ are computed by ŝᵤ[l]=(\hat{d}ᵤ[G(l+1)]−\hat{d}[Gl])/Tₛ. The goal is to retrieve ŝᵤ^{w} and ŝᵤ^{v} from ŝᵤ, where the assumptions (A-2), (A-3) and (A-4) are used.

The matrix form of Eq. (59) can be written as ∥W_{F}Ŝᵤ^{v}∥₂²≈0 where W_{F}∈ [symbol] ^{K×L} is constructed from the DFT matrix and each element is

$${W}_{F} ( k , l ) = exp ( - j 2 π \frac{k}{L} l )$$

with K<(L−1)/2 and k/(TₛL)∉B. From (A-3), the smoothness can be represented by ∥Dŝᵤ^{v}∥₂², where D∈ [symbol] ^{(L-1)×L} is the matrix form of a differential operator, i.e.,

$$D = [ \begin{matrix}- 1 & 1 & 0 & … & 0 \\ 0 & - 1 & 1 & … & 0 \\ ⋮ & ⋮ & ⋮ & ⋱ & ⋮ \\ 0 & 0 & 0 & … & - 1\end{matrix} ] \qquad ( 63 )$$

In (A-4), the sparsity can be represented as ∥Dŝᵤ^{w}∥₁. Based on the above assumptions and Eq. (52), the following constrained optimization problem is formulated:

$${min}_{{\hat{s}}_{u}^{w} {\hat{s}}_{u}^{u}} \{ {α}_{0} {‖ {W}_{F} {\hat{s}}_{u}^{v} ‖}_{2}^{2} + {α}_{1} {‖ D {\hat{s}}_{u}^{v} ‖}_{2}^{2} + {α}_{2} {‖ D {\hat{s}}_{u}^{w} ‖}_{1} \} s . t . {\hat{s}}_{u}^{w} + {\hat{s}}_{u}^{v} = {\hat{s}}_{u} , \qquad ( 64 )$$

where α_{0,1,2} are regularization coefficients. To solve Eq. (64), the augmented Lagrangian [symbol] is introduced:

$$ℒ ( {\hat{s}}_{u}^{w} , {\hat{s}}_{u}^{v} , λ ) = {α}_{0} {‖ {W}_{F} {\hat{s}}_{u}^{v} ‖}_{2}^{2} + {α}_{1} {‖ D {\hat{s}}_{u}^{v} ‖}_{2}^{2} + {α}_{2} {‖ D {\hat{s}}_{u}^{w} ‖}_{1} + {‖ {\hat{s}}_{u} - {\hat{s}}_{u}^{w} - {\hat{s}}_{u}^{v} ‖}_{2}^{2} + 〈 λ , {\hat{s}}_{u} - {\hat{s}}_{u}^{w} - {\hat{s}}_{u}^{v} 〉 , \qquad ( 65 )$$

where λ is the Lagrangian multiplier. We use alternating direction method of multipliers (ADMM) is used [21] to solve Eq. (65) iteratively. In the i-th iteration, it contains three steps:
(A) Minimization w.r.t ŝᵤ^{w}:

$${\hat{s}}_{u}^{w} ( i + 1 ) = {argmin}_{{\hat{s}}_{u}^{w}} \{ {α}_{2} {‖ D {\hat{s}}_{u}^{w} ‖}_{1} + {‖ {\hat{s}}_{u} - {\hat{s}}_{u}^{w} - {\hat{s}}_{u}^{v} ( i ) + \frac{λ ( i )}{2} ‖}_{2}^{2} \} , \qquad ( 66 )$$

(B) Minimization w.r.t Ŝᵥ^{v}:

$${\hat{s}}_{u}^{v} ( i + 1 ) = {argmin}_{{\hat{s}}_{u}^{v}} \{ {α}_{0} {‖ {W}_{F} {\hat{S}}_{u}^{v} ‖}_{2}^{2} + {α}_{1} {‖ D {\hat{s}}_{u}^{v} ‖}_{2}^{2} + {‖ {\hat{s}}_{u} - {\hat{s}}_{u}^{w} ( i ) - {\hat{s}}_{u}^{v} + \frac{λ ( i )}{2} ‖}_{2}^{2} \} \qquad ( 67 )$$

(C) Update λ:

$$λ ( i + 1 ) = λ ( i ) + β ( {\hat{s}}_{u} - {\hat{s}}_{u}^{w} ( i + 1 ) - {\hat{s}}_{u}^{v} ( i + 1 ) ) , \qquad ( 68 )$$

where (⋅) denotes the iteration step, and β is update parameter. After ŝᵤ^{v} is obtained, according to Eq. (51), HR can be estimated from ŝᵤ^{v} according to the VSDR proposed above, with regards to the static model.

In the following the method is validated with simulated examples that combine the proposed model of Eq. (58) with in-vivo impedance data from [19]. Specifically, when simulating the data, the inventors consider a realistic scenario where each chest is considered to be shaped by multiple close scatterers moving in phase but with different motion amplitudes. In addition, the scatterers' locations are corrupted with time-varying noise to mimic the body micro-movement. In the solution, it is still assumed that each person is represented by one scatterer.

The radar parameters are based on Texas Instruments IWR1642 76 to 81 [GHz] mmWave sensor, which corresponds to a center frequency of 77 [GHz], T_{c}=57 [μs] and S=70 [MHz/μs], a bandwidth of B≈4 [GHz], and f_{ADC}=4 [MHz], with selected N=200. A frame rate of 500 [Hz] is adopted, meaning T_{g}=2 [ms], and M=100. The window size is selected as G=50, leading to a window period Tₛ=100 [ms].

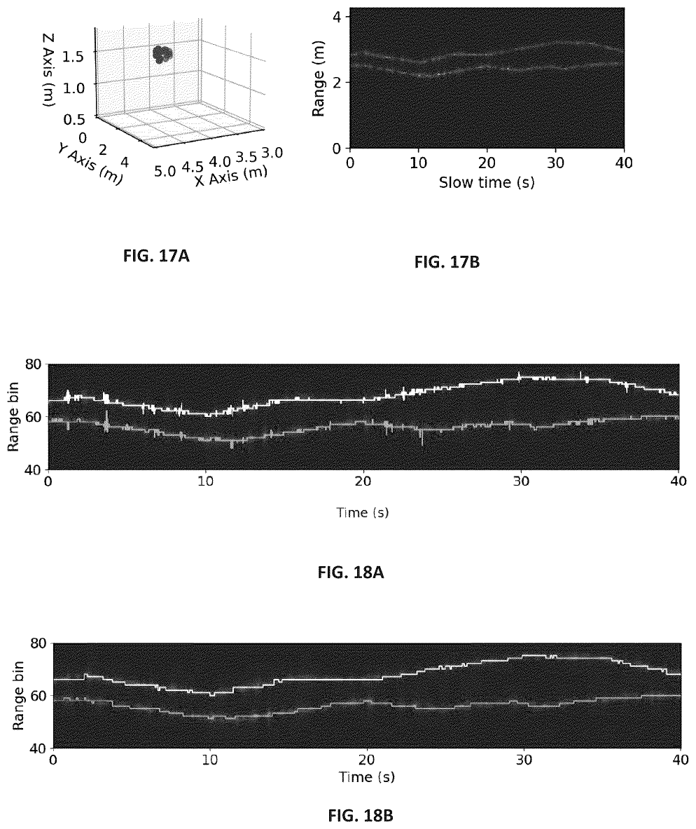

The inventors consider two subjects separated in range. The starting distance at τ=0 is 2.5 [m] and 2.8 [m], respectively. In the simulation, 20 scatterers are used with random amplitudes to represent each chest, the coordinates of which are uniformly distributed. A realization of one cluster of a person is shown in FIG. 17A. To mimic the body micro-movement, Gaussian noises are sampled with a time interval 0.4 [s] and a standard deviation 0.5 [mm], interpolated on the slow time grid, and added to the time-varying distance of all scatterers. The distances of scatterers are combined with chest vibration distances taken from [19]. Next, beat signals are simulated using the distances of all scatterers. Finally, Gaussian noises are added to the beat signals, resulting in an SNR of 8. FIG. 17B is the range-FFT map, showing the moving distance within a monitoring time of 40 [s]. Notice that different accelerations are intentionally added to the closer subject (bottom trajectory) to test the method's adaptability. The true HRs for the two subjects are 71.8 and 64.4 BPM, respectively.

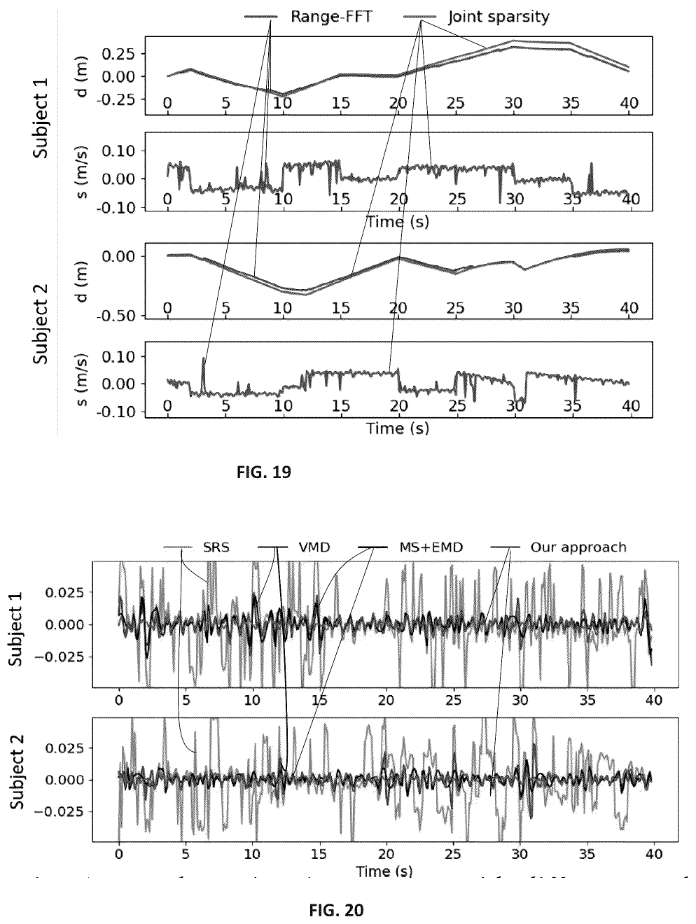

The localized range bins are drawn on the range-FFT map, shown in FIGS. 18A and 18B. For comparison, FIG. 18A shows the bins directly selected from the range-FFT. The trajectories have many jumps due to environmental noises and movement oscillations of multiple scatterers. In contrast, the joint sparsity supports are more continuous and reasonable, see FIG. 18B. Quantitively, based on range-FFT, the mean absolute error (MAE) between the localized and true range for the two subjects is 1.5 [cm] and 1.4 [cm], respectively, with the maximum error (ME) being 28 [cm] and 14 [cm], respectively. In joint sparsity recovery, the MAE decreases to 1.4 [cm] and 1.3 [cm], and the ME is reduced to 4.2 [cm] and 4.7 [cm], respectively. FIG. 19 shows the estimated distances from phase and the corresponding velocity of the two subjects based on range-FFT or joint sparsity recovery. For each subject, the first and second row is the estimated distance and velocity, respectively. It can be observed that the chest vibration is indistinguishable in distance but becomes significant in velocity. In addition, the estimated distance and velocity based on range-FFT exhibit many jumps due to incorrect selection of range bins, while the joint sparsity recovery overcomes these issues.

For vital sign extraction, the inventors use the results obtained from joint sparsity recovery (red lines in FIG. 19) to compare different methods. The first comparison removes movement distance using the smoothed range estimated from the fast time frequency, referred to as smoothed range subtraction (SRS), which follows the first step from [24]. The second comparison is performing variational mode decomposition (VMD) on the estimated distance, based on [25]. The third comparison is sequentially performing mean subtraction (MS) [26] and empirical mode decomposition (EMD) on the estimated phase, inspired by [24]. This method is referred to herein as MS+EMD. FIG. 20 shows the results of the approach of the present disclosure and these comparisons. Overall, the heartbeat velocity from the approach of the present disclosure is less affected by large movements compared to other methods. Table 6 presents the estimated HR from the extracted heartbeat signal using the VSDR for all 40 seconds. The results match the reference HR well. The average HR estimation error from the four methods is 20.7, 18.7, 4.5, and 0.5 BPM, respectively. Interestingly, for subject 2 with movement accelerations, the extraction still shows promising results, even though the above assumption (A-4) does not account for this scenario.

TABLE 6

| Subject | SRS | VMD | MS + EMD | Ours | True HR |
|---|---|---|---|---|---|
| 1 | 43.3 | 70.9 | 70.7 | 70.9 | 71.8 |
| 2 | 77.3 | 100.8 | 72.2 | 64.2 | 64.4 |

In the following, the inventors introduce a comprehensive framework for multi-person localization and NCVSM using MIMO ULA FMCW radar, addressing critical challenges in cluttered, real-world environments. The inventors designed a novel custom hardware phantom capable of accurately replicating the cardiopulmonary dynamics of multiple individuals. This phantom provides a realistic and repeatable testbed for validating radar systems and algorithms, bridging the gap between theoretical development and practical deployment in clinical and home-care settings. Additionally, leveraging insights from the phantom validation process, the inventors developed two novel algorithms: the RaLU-JSR method for multi-person localization, which exploits joint sparsity and cardiopulmonary properties, and the E-VSDR approach for continuous, harmonics resilient vital signs estimation, effectively mitigating interference from respiration harmonics through a tailored dictionary-based method. The proposed framework was rigorously evaluated through both phantom trials and human trials, with results demonstrating the potential of the framework to advance NCVSM by providing robust, and accurate monitoring solutions, even in complex, cluttered environments. These findings offer a transformative step toward practical radar-based healthcare monitoring.

In the following, the signal model is described.

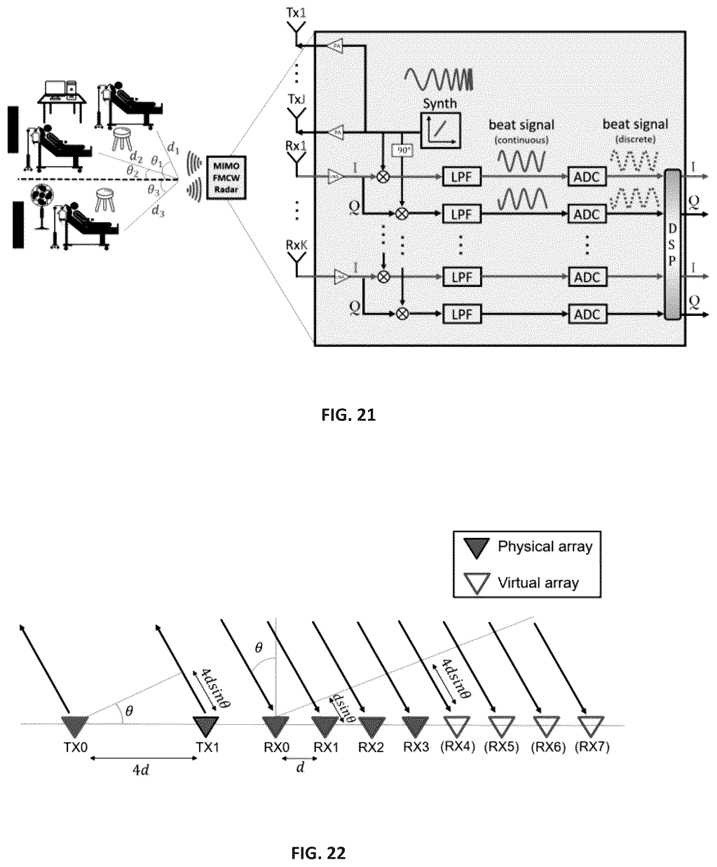

Consider an FMCW radar in a MIMO ULA setup containing/≥1 transmitters and K≥1 receivers, as illustrated in FIG. 21. The j'th transmitter emits L frames of chirp signals at a frame rate of fₛ, designed to localize and monitor the vital signs of Z≥1 humans within a cluttered environment containing U≥Z objects distributed across various angular and radial positions relative to the radar antennas. The k'th receiver captures the reflected echoes, which are separated into the I/Q channels, mixed with the transmitted signal, and sampled by an ADC with interval T_{f} to produce discrete baseband (beat) signals of length N [11] [15]. The amplitudes of these signals are attenuated based on the radar cross-section (RCS) of the reflecting objects.

For a single transmitter and K receivers located at rₖ≙(k−1)λ/2, k=1, . . . , K, where λ is the chirp's maximal wavelength, the 3D SIMO FMCW beat signal model representing U objects in the radar's FOV can be expressed as:

$$y [ n , k , l ] = {\sum }_{u = 1}^{U} {x}_{u} {e}^{j ( 2 π {f}_{u} n {T}_{f} + \frac{2 π}{λ} {r}_{k} {sin θ}_{u} + {ψ}_{u} [ l ] )} + w [ n , k , l ] , \qquad ( 69 )$$

for n=1, . . . , N fast-time samples, k=1, . . . , K receivers and l=1, . . . , L slow-time frames. Here, {w[n, k, l]} is a 3D sequence of zero mean i.i.d. complex Gaussian noise with variance σ². The received signal is comprised of U components where the u'th component is characterized by four parameters: (i) a constant amplitude xᵤ, related to the RCS of the u'th object; (ii) a beat frequency fᵤ which is proportional to the u'th object's radial distance dᵤ by

$${f}_{u} {=}^{Δ} \frac{2 S}{c} {d}_{u} ,$$

where c is the speed of light and S≙B/T_{c} corresponds radial distance du by to the rate of the frequency sweep with B and T_{c} denoting the chirp's total bandwidth and duration, respectively; (iii) an azimuth angle θᵤ; and (iv) a slow-time varying phase term ψᵤ[l] that tracks small vibrations of the u'th object.

Assuming that each object has a distinct pair of distance and angle {dᵤ, θᵤ}, the model in Eq. (69) can be rewritten for M≥U general radial distances {dₘ}ₘ₌₁^{M} and P≥U general azimuth angles {θₚ}ₚ₌₁^{P} as:

$$y [ n , k , l ] = {\sum }_{p = 1}^{P} {\sum }_{m = 1}^{M} {x}_{m , p} {e}^{j ( {ω}_{m} [ n ] + {ϕ}_{p} [ k ] + {ψ}_{m , p} [ l ] )} + w [ n , k , l ] , \qquad ( 70 )$$

where each {m, p} component is associated with reflection from a different distance-angle pair {dₘ, θₘ}, including reflections from Z monitored persons. Based on the latter, x_{m,p} denotes the beat amplitude of the {m, p}'th component, which can be zero if there is no reflection and includes the unknown amplitudes related to the RCS of the monitored individuals. The fast-time modulated function ωₘ[n] is defined by:

$${ω}_{m} [ n ] {=}^{Δ} 2 π {f}_{m} n {T}_{f} , n = 1 , … , N , \qquad ( 71 )$$

where each beat frequency fₘ is distinct and proportional to a different radial distance from the radar dₘ by

$${f}_{m} {=}^{Δ} \frac{2 S}{c} {d}_{m} , m = 1 , … , M \leq N \qquad ( 72 )$$

where the Z unknown human distances {d^{(z)}}_{z=1}^{Z} are included in the distances {dₘ}ₘ₌₁^{M}. The azimuth angles {θₚ}ₚ₌₁^{P} are reflected in the following phase shifts due to the ULA antenna geometry:

$${ϕ}_{p} [ k ] = \frac{2 π}{λ} {r}_{k} sin {θ}_{p} , k = 1 , … , K \leq P , \qquad ( 73 )$$

where the Z unknown human angles {θ^{(z)}}_{z=1}^{Z} are among the angles {θₚ}ₚ₌₁^{P}. Finally, the slow-time varying term ψ_{m,p}[l] of each component is given by:

$${ψ}_{m , p} [ l ] {=}^{Δ} \frac{4 π}{λ} ( {d}_{m} + {v}_{m , p} [ l ] ) , l = 1 , … , L , \qquad ( 74 )$$

where the vibration function v_{m,p}[l] is generally modeled for both human and clutter objects by

$${v}_{m , p} [ l ] {=}^{Δ} {\sum }_{q = 1}^{Q} {a}_{m , p}^{( q )} cos ( 2 π {g}_{m , p}^{( q )} l {T}_{s} ) , l = 1 , … , L \geq Q . \qquad ( 75 )$$

The pairs {a_{m,p}^{(a)}, g_{m,p}^{(q)}}_{q=1}^{Q} are the corresponding amplitudes and frequencies, with the latter being limited by the slow-time frame rate fₛ≙1/Tₛ according to {gₘ^{(q)}}_{q=1}^{Q}∈[0fₛ/2] for each {m, p} component. The generalized vibration model in Eq. (75) enables effective representation of both static and vibrating objects, including harmonic components, through suitable choices of {a_{m,p}^{(q)}}_{q=1}^{Q} and {g_{m,p}^{(q)}}_{q=1}^{Q}.

In the present disclosure, the inventors investigate multi-person NCVSM of an unknown number of people Z. Their thoracic vibrations, which are included in {v_{m,p}[l]}, are denoted by {v^{(z)}[l]}_{z=1}^{Z}, and satisfy

$${v}^{( z )} [ l ] {=}^{Δ} {\sum }_{q = 1}^{Q} {a}_{q}^{( z )} cos ( 2 π {g}_{q}^{( z )} l {T}_{s} ) , l = 1 , … , L , \qquad ( 76 )$$

with amplitudes {a_{q}^{(z)}}_{q=1}^{Q} and a frequency set {g_{q}^{(z)}}_{q=1}^{Q} that includes the unknown Z pairs of HR and RR, denoted by {f_{H}^{(z)}, f_{R}^{(z)}}_{z=1}^{Z}.

In the following, based on the signal model presented above, the problem formulation is presented through a simplified matrix representation to facilitate the analysis. Specifically, by defining the complex amplitudes

$${\tilde{x}}_{m , p} [ l ] {=}^{Δ} {x}_{m , p} {e}^{j {ψ}_{m , p} [ l ]} , l = 1 , … , L , \qquad ( 77 )$$

the received samples from Eq. (70) can be arranged into the matrix Yₗ∈ [symbol] ^{N×K} for each frame. This leads to the model:

$${Y}_{l} = A {X}_{l} B + {W}_{l} , l = 1 , … , L \qquad ( 78 )$$

where A∈ [symbol] ^{N×M} is a known range-related Vandermonde matrix, whose entries are given by A(n, m)≙e^{jω}^{ₘ}^{[n]} (See Eq. (71)), B∈ [symbol] ^{P×K} is a known angle-related matrix whose entries are given by B(p, k)≙e^{jϕ}^{_{P}}^{[k]} (See Eq. (73)), Xₗ∈ [symbol] ^{M×P} is an unknown matrix of complex amplitudes where Xₗ(m, p)≙\tilde{x}_{m,p}[l] (See Eq. (77)), and Wₗ∈ [symbol] ^{N×K} is the noise matrix where Wₗ(n, k)≙w[n, k, l] (See Eq. (69)). It is noted that while the model in Eq. (78) is designed for a SIMO ULA setup, it is also applicable to a MIMO ULA by employing orthogonal transmission schemes, such as TDM [27]. This approach effectively creates a virtual SIMO ULA with a number of virtual receivers equal to the product of the transmitters with the receivers, as illustrated in FIG. 22, thereby significantly enhancing angular resolution.

To enable continuous vital signs monitoring, the FMCW radar periodically transmits, receives, and processes frames of chirp signals throughout the monitoring session. At each predefined time interval Tᵢₙₜ, the sequence {Yₗ}ₗ₌₁^{L} in Eq. (78) is constructed by aggregating all L frames recorded within the preceding time window T_{win}, where L is given by L=T_{win} fₛ. The data model in Eq. (78) is assumed to exhibit the following properties:
B-1 The monitored individuals remain stationary, with only slight thoracic movements due to cardiopulmonary activity. As a result, the {m, p} coordinates in {Xₗ}ₗ₌₁^{L}, corresponding to their locations {d^{(z)}, θ^{(z)}}_{z=1}^{Z}, are fixed and joint across all L frames.
B-2 The number of objects within the radar's FOV satisfies U<<MP, meaning that {Xₗ}ₗ₌₁^{L} are U-sparse matrices.
Based on the model in Eq. (78), the first goal is to estimate the number of individuals, Z, along with their spatial locations {d^{(z)}, θ^{(z)}}_{z=1}^{Z} (See Eqs. (72), (73)), by recovering {Xₗ}ₗ₌₁^{L} and identifying the corresponding {m, p} indices. Subsequently, the second objective is to continuously monitor each detected individual's HR and RR by extracting their thoracic vibrations {v^{(z)}[l]}_{z=1}^{Z} (See Eq. (76)) encoded in {Xₗ}ₗ₌₁^{L}, and estimating the vital pairs {f_{H}^{(z)}, f_{R}^{(z)}}_{z=1}^{Z} at each Tᵢₙₜ. It is noted that the latter stage is supported by a dedicated matrix representation as detailed further below.

Aided by the designed phantom described further below, the inventors developed a robust algorithm for multi-person localization and vital signs monitoring in real-world, cluttered environments, employing an FMCW radar in either a SIMO ULA or a MIMO ULA setup. In the following, each stage of the proposed approach is detailed, based on the model presented above.

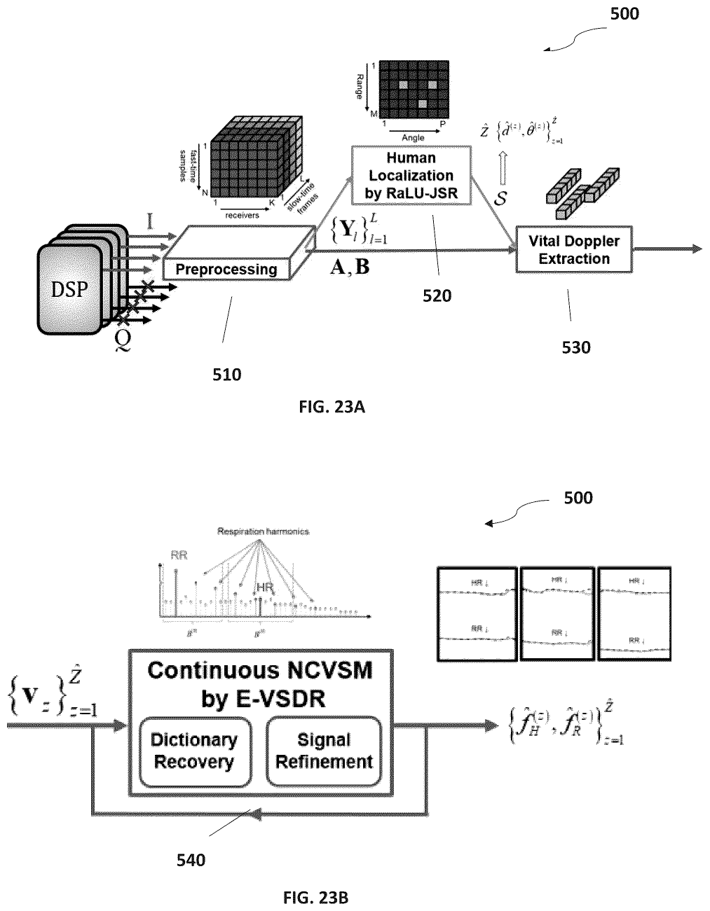

Reference is made to FIGS. 23A and 23B showing by block diagram 500 the proposed algorithm for robust multi-person localization and vital signs monitoring using SIMO or MIMO ULA FMCW Radar.

The first step (step 510 in FIG. 23A) of each monitoring iteration involves preliminary processing of the radar channel data to construct the 3D cube {Yₗ}ₗ₌₁^{L} that satisfies the model in Eq. (78) with a high SNR. The subsequent step entails assembling the known matrices A and B based on predefined frequency and angle grids.

Recall that each element of {Wₗ}ₗ₌₁^{L} in Eq. (78) comes from a zero-mean i.i.d. Gaussian distribution. Hence, similarly to the method described above, the noise variance is reduced by utilizing the slowness of thoracic movement relative to the frame period Tₛ [11]. To this end, G>1 consecutive chirps are transmitted at each frame and coherently combined to produce a frame with a single chirp that satisfies Eq. (78) with variance smaller by a factor of G, w.r.t. transmission of only a single chirp per frame.

Next, the dictionary matrix A is set by assuming that the distance-related frequencies {fₘ}ₘ₌₁^{M} (Eq. (72)) lie on the Nyquist grid, i.e.,

$${f}_{m} = \frac{{f}_{A D C}}{N} {i}_{m} , {i}_{m} = 0 , … , M - 1 , \qquad ( 79 )$$

where f_{ADC}≙1/T_{f} is determined by the ADC component. It is noted, using Eqs. (77) and (69), that the maximal detectable distance is

$${d}_{max} = \frac{c {f}_{A D C}}{2 SN} ( M - 1 ) ,$$

and the range resolution is

$${d}_{res} = \frac{c {f}_{A D C}}{2 S N} = \frac{c}{2 B}$$

since N=f_{ADC}T_{c} and ST_{c}=B. Using Eq. (79) and since A(n, m)≙e^{j2πf}^{ₘ}^{nT}^{_{f}}, it can be stated that

$$A ( n , m ) = {e}^{j 2 π \frac{L m}{N} n} .$$

As for the dictionary matrix B, the phase shifts {ϕₚ[k]}ₖ₌₁^{K} (δ) are set to cover a FOV of 180[°] according to the following angle grid:

$${θ}_{p} = - 90 + {i}_{p} {Δ}_{θ} , {i}_{p} = 0 , … , P - 1 , P = \frac{180}{{Δ}_{θ}} , \qquad ( 80 )$$

where Δ_{θ} denotes the spacing of the angle grid. Then, by Eqs. (73), (80) and since B(p,k)≙e^{jϕ}^{ₚ}^{[k]}, it can be stated that B(k, p)=e^{jπ(k-1)sin(-90+i}^{ₚ}^{Δ}^{_{θ}}⁾.

To estimate the number of humans Z and their spatial location {d^{(z)}, θ^{(z)}}_{z=1}^{Z} (step 520 in FIG. 23A), the assembled dictionaries A and B are used, as well as {Yₗ}ₗ₌₁^{L} for the first L=T_{loc}fₛ frames, where T_{loc} denotes the duration of localization. Since the goal of the technique of the present disclosure is the continuous monitoring of multiple stationary subjects, {Xₗ}ₗ₌₁^{L} and the corresponding support are recovered only once. Here, the support is denoted by S and defined as the set of 2D{m, p} indices whose cardinality corresponds to the number of individuals Z and whose indices point to their respective range-angle locations {d^{(z)}, θ^{(z)}}_{z=1}^{Z} by Eqs. (72), (79) and (80). The approach of the present technique removes the need to recover {Xₗ}ₗ₌₁^{L} and [symbol] at each monitoring iteration, as further explained below.

To this end, the inventors start with clutter suppression utilizing prior knowledge of human-typical pulse and breathing frequencies by extending the vital-based spectral filter introduced above for a single receiver, here to the case of K>1 receivers. The data of each k'th antenna is selected by defining Y^{(k)}∈ [symbol] ^{N×L}: Y^{(k)}(n, l)≙Yₗ(n, k) (i). Then, each filtered matrix is given by

$${\overline{Y}}^{( k )} = \frac{1}{L} {( {F}_{L}^{H} ( \prod ⊙ {F}_{L} {Y}^{( k ) T} ) )}^{T} , k = 1 , … , K \qquad ( 81 )$$

where F_{L} is a full L-size DFT matrix, Π∈ [symbol] ^{L} denotes a window function corresponding to the vital frequencies in B^{(H)}∪B^{(R)} with B^{(R)} and B^{(H)} respectively denoting the frequency bands of respiration and heartbeat at rest, and ⊙ denotes the element-wise product. The filtered data is then reshaped back to the original structure as in Eq. (78) denoted by {Y̅ₗ}ₗ₌₁^{L}.

Next, by assumptions A-1 and A-2 the U-sparse matrices {Xₗ}ₗ₌₁^{L} share joint support. Hence, the inventors propose to recover them from {Y̅ₗ}ₗ₌₁^{L} by promoting the joint sparsity via the following 3D l_{2,1}-norm regularized Least-Squares (LS) problem given A and B:

$$\hat{𝕏} = {argmin}_{𝕏 ∈ {ℂ}^{M \times P \times L}} \frac{1}{2 L} {\sum }_{l = 1}^{L} {‖ {\overline{Y}}_{l} - A {X}_{l} B ‖}_{F}^{2} + γ {‖ 𝕏 ‖}_{2 , 1} , \qquad ( 82 )$$

where ∥ [symbol] ∥_{2,1} refers to the sum of all l₂ norms over the frame (third) dimension of the 3D tensor [symbol] ∈ [symbol] ^{M×P×L} which concatenates the frames of {Xₗ}ₗ₌₁^{L} and γ≥0 is the regularization parameter. To solve Eq. (82) the inventors extend the JSR algorithm proposed in [18], which is based on the fast iterative soft-thresholding algorithm (FISTA), to accommodate the proposed bilinear formulation Eq. (78). The method is called RaLU-JSR: Radar Localization of hUmans via Joint Sparse Recovery and is given in Algorithm 1.

Algorithm 1: RaLU-JSR for minimizing Eq.(82)

```
Input: {Y̅ₗ}ₗ₌₁^{L}, A, B, L_{f}, γ > 0, I
Initialize: i = 1, t⁽¹⁾ = 1, {Zₗ⁽¹⁾ = Xₗ⁽⁰⁾ = 0^{M×P}}ₗ₌₁^{L}
  while i < Iₘₐₓ or stopping criteria not fulfilled do:
  $1 : {G}_{l}^{( i )} = {Z}_{l}^{( i )} - \frac{1}{{L}_{f}} {A}^{H} ( {AZ}_{l}^{( i )} B - {Y}_{l} ) {B}^{H} , l = 1 , … , L$
  $2 : {\{ {X}_{l}^{( i + 1 )} \}}_{l = 1}^{L} = {𝒯}_{\frac{γ}{{L}_{f}}}^{( 3 D )} ( {\{ {G}_{l}^{( i )} \}}_{l = 1}^{L} )$
  3: t⁽ⁱ⁺¹⁾ = 0.5 (1 + {square root over (1+ 4t⁽ⁱ⁾²))}
  $4 : {Z}_{l}^{( i + 1 )} = {X}_{l}^{( i )} + \frac{{t}^{( i )} - 1}{{t}^{( i + 1 )}} ( {X}_{l}^{( i )} - {X}_{l}^{( i - 1 )} ) , l = 1 , … , L$
  5: i ← i + 1
  end while
  Return: {Xₗ^{(l)}}ₗ₌₁^{L}
```

In Algorithm 1, I denotes the maximal number of iterations, L_{f} denotes the Lipschitz constant. L_{f}=λₘₐₓ (A^{H}A)λₘₐₓ (B^{H}B) with λₘₐₓ denoting the largest singular value. [symbol] _{α}^{(3D)} (⋅) is the 3 D soft-threshold operator such that for each {m, p}'th element of Xₗ⁽ⁱ⁺¹⁾, l=1, . . . , L, it can be written:

$${X}_{l}^{( i + 1 )} ( m , p ) = max ( 0 , 1 - α / {‖ {𝔾}^{( i )} ( m , p ) ‖}_{2} ) {G}_{l}^{( i )} ( m , p ) , \qquad ( 83 )$$

where the tensor [symbol] ⁽ⁱ⁾∈ [symbol] ^{M×P×L} concatenates all L matrices Gₗ⁽ⁱ⁾∈ [symbol] ^{M×P}, l=1, . . . , L, as detailed in Algorithm 1.

The support [symbol] is estimated by first taking the average power across the frames of {Xₗ^{(I)}}ₗ₌₁^{L} which results in a 2D range-angle localization map, denoted by X̅∈ [symbol] ^{M×P}. Then, various methods can be used to determine [symbol] from X̅, (and correspondingly Z, and {\hat{d}^{(z)}, \hat{θ}^{(z)}}_{z=1}^{\hat{Z}}), such as CA-CFAR [28] or 2D peak-detection. It is noted that predetermined boundaries of range and angle can serve as a focused region of interest (ROI) in X̅ for evaluating [symbol] .

The support evaluated in the localization step allows the inventors to efficiently recover only the Doppler samples related to human vital signs concealed in the input set {Yₗ}ₗ₌₁^{L} (i) of each monitoring iteration (step 530 in FIG. 23A). Particularly, using [symbol] we estimate the complex amplitudes {\tilde{x}_{m,p}[l]}ₗ₌₁^{L} (9) of each z'th human, denoted by {x_{S(z)}[l]}ₗ₌₁^{L} using the following beamformer over the support:

$${\hat{x}}_{S ( z )} [ l ] = \frac{1}{NK} {A}_{S ( z )}^{H} {Y}_{l} {B}_{S ( z )}^{H} , l = 1 , … , L \qquad ( 84 )$$

for each human z=1, . . . , \hat{Z}, where A_{S(z)}∈ [symbol] ^{N×1} and B_{S(z)}∈ [symbol] ^{1×K} respectively denote the atoms of A and B corresponding the z'th 2 D index of [symbol] . We note that since A(n, m)=

$${e}^{j 2 π \frac{2 m}{N} n} ,$$

A_{S(z)}^{H} equals to the row of a partial DFT matrix corresponding to the fast-time frequency fₘ (11) for m∈ [symbol] (z). Hence, analogous to technique described above, by selecting M=N/2 in (11) (i.e. considering only non-negative fast-time frequencies), the estimator in (16) given {Yₗ}ᵢ₌₁^{L} (10) assembled by both the I and Q channels, is comparable to that obtained by simply using a single channel (I or Q), up to a constant factor. To minimize hardware overload and possible concerns of using two channels concurrently, we assume M=N/2 and use only the In-Phase channel to construct (10).

Next, since \hat{x}_{S(z)}[l] (16) estimates the phasor terms x_{m,p}e^{jψ}^{_{m,p}}^{[l]} (9) for {m, p}∈ [symbol] , that modulate the thoracic vibrations {v^{(z)}[l]}ᵢ₌₁^{L} (6)-(8), we extract the phase of each z'th human using an arctangent demodulation-based method similarly to [18], [19]:

$${\hat{v}}^{( z )} [ l ] = unwrap ( ∠ ( {\hat{x}}_{S ( z )} [ l ] ) ) = 1 , … , L , \qquad ( 85 )$$

where unwrap(⋅) denotes the unwrapping procedure described in [11], used since the unambiguous phase range is limited by (−ππ] and the angle extraction operator ∠(⋅) is based on the four-quadrant arctangent function. For convenient analysis, the estimated samples are then concatenated into the vector \hat{v}_{z}∈ [symbol] ^{L} which represents a scaled approximation of {v^{(z)}[l]}ᵢ₌₁^{L} in Eq. (76), for each z'th detected human, z=1, . . . , \hat{Z}.

In the final stage of each monitoring iteration, the vital signs of the detected individuals, {f_{H}^{(z)}, f_{R}^{(z)}}_{z=1}^{Z}, are estimated given the extracted vibrations {\hat{v}_{z}}_{z=1}^{\hat{Z}} (Eq. (85)), and recorded for continuous NCVSM (step 540 in FIG. 23B).

In the following, the inventors present an extension to the VSDR approach introduced above, termed E-VSDR, which is tailored for continuous monitoring in low-SNR conditions where interfering harmonics due to the non-pure sinusoidal nature of human thoracic motion pose challenges. Furthermore, E-VSDR incorporates a dedicated adaptive signal refinement procedure that leverages the high rate of output estimates to enhance monitoring accuracy, making it particularly effective for continuous NCVSM in real-world, cluttered environments.

First, according to Eq. (76), and given the non-overlapping nature of respiration and heartbeat frequency bands, the extracted vibrations {\hat{v}_{z}}_{z=1}^{\hat{Z}} (Eq. (85)) can be described by

$${\hat{v}}_{z} = {D}^{( R )} {a}_{z}^{( R )} + {D}^{( H )} {a}_{z}^{( H )} + {n}_{z} , z = 1 , … , \hat{Z} \qquad ( 86 )$$

where D^{(R)}∈ [symbol] ^{L×Q}^{_{R}} and D^{(H)}∈ [symbol] ^{L×Q}^{_{H}}, Q_{R}, Q_{H}<Q respectively denote the respiration and heartbeat dictionaries:

$${D}^{( R )} ( l , q ) \hat{=} cos ( 2 π {g}_{q}^{( R )} {lT}_{s} ) and \qquad ( 87 )$$

$${D}^{( H )} ( l , q ) \hat{=} cos ( 2 π {g}_{q}^{( H )} {lT}_{s} ) ,$$

where {g_{q}^{(R)}}_{q=1}^{Q}^{_{R}}∈B^{(R)} and {g_{q}^{(H)}}_{q=1}^{Q}^{_{H}}∈B^{(H)} with B^{(R)}∩B^{(H)}=Ø. The amplitude vectors a_{z}^{(R)}∈ [symbol] ^{Q}^{_{R}} and a_{z}^{(H)}∈ [symbol] ^{Q}^{_{H}} control the frequency pattern of each z'th human vibration through D^{(R)} and D^{(H)}. Finally, {n_{z}}_{z=1}^{\hat{Z}} denote length-L sequences of i.i.d. noise vectors as a result of the non-linear operations up to Eq. (85).

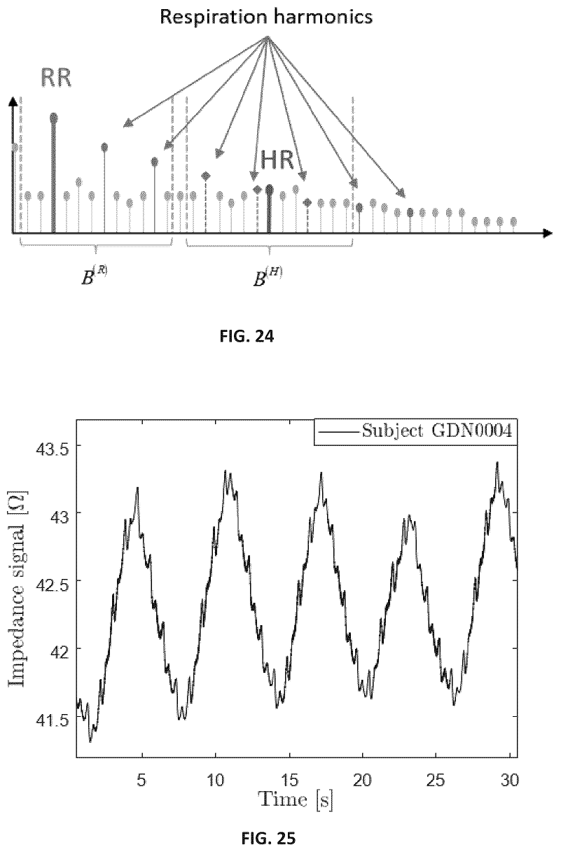

Next, to mitigate the phenomenon where respiration harmonics may mask the heartbeat tone in B^{(H)} (see FIG. 24), for each z'th human the inventors divide the frequencies in B^{(H)} into two distinct groups:
1. Interfering respiration harmonics, denoted by {g_{q}^{(R′,z)}}_{q=1}^{Q}^{_{R′,z}}, defined as multiples of the fundamental respiration frequency f_{R}^{(z)} that reside in B^{(H)}:

$$\{ {g}_{q}^{( {R}^{′} , z )} = {g}_{q}^{( H )} : {g}_{q}^{( H )} = {i}_{q} {f}_{R}^{( z )} , {i}_{q} {f}_{R}^{( z )} ∈ {B}^{( H )} \} . \qquad ( 88 )$$

2. Non-interfered heart frequencies, denoted by {g_{q}^{(H′,z)}}_{q=1}^{Q}^{_{H}}^{,z} which are the complementary frequencies to {g_{q}^{(R′,z)}}_{q=1}^{Q}^{_{R′,z}} in B^{(H)} that include the true HR, f_{H}^{(z)}. Consequently, {g_{q}^{(R′,z)}}_{q=1}^{Q}^{_{R′,z}} and {g_{q}^{(H′,z)}}_{q=1}^{Q}^{_{H′,z}} respectively compose the dictionaries and D^{(R′,z)}∈ [symbol] ^{L×Q}^{_{R′,z}} and D^{(H′,z)}∈ [symbol] ^{L×Q}^{_{H′,z}} similarly to Eq. (87), where D^{(R′,z)}∪D^{(H′,z)}=D^{(H)} and Q_{H′,z}+Q_{R′,z}=Q_{H}. It is noted that the dictionaries D^{(R′,z)} and D^{(H′,z)} are unknown a priori, as they rely on the unknown respiratory fundamental frequency of each individual, f_{R}^{(z)}. Under these settings, the z'th extracted vibration vector \hat{v}_{z} (Eq. (86)) can be represented as

$${\hat{v}}_{z} = {D}^{( R )} {a}_{z}^{( R )} + {D}^{( {R}^{′} , z )} {a}_{z}^{( {R}^{′} )} + {D}^{( {H}^{′} , z )} {a}_{z}^{( {H}^{′} )} + {n}_{z} \qquad ( 89 )$$

for z=1, . . . , \hat{Z}, where a_{z}^{(R′)}∈ [symbol] ^{Q}^{_{R′,z}} and a_{z}^{(H′)}∈ [symbol] ^{Q}^{_{H′,z}} respectively denote the corresponding amplitudes of interfered and non-interfered heartbeat tones. Given the model in Eq. (89), and the prominence of respiration and heartbeat tones in cardiopulmonary activity, the amplitude vectors a_{z}^{(R)} and a_{z}^{(H′)} are assumed to be 1-sparse vectors.

Finally, to allow for short estimation windows but with sufficient frequency resolution, similarly to the technique described above, the inventors uniformly split the slow-time frequency segment [0, fₛ/2), (which includes B^{(R)} and B^{(H)}) according to a resolution of 1 bpm. This means that given a dense grid of frequencies

$${g}_{q} = {h}_{q} \frac{{f}_{s}}{Q} , {h}_{q} = 0 , … , \frac{Q}{2} - 1 , Q = 60 {f}_{s} , \qquad ( 90 )$$

the frequencies and {g_{q}^{(R)}}_{q=1}^{Q}^{_{R}} and {g_{q}^{(H)}}_{q=1}^{Q}^{_{H}} of (19), constitute subsets of Eq. (90) according to the limits defined by B^{(R)} and B^{(H)}, respectively. These frequencies are then used to assemble D^{(R)} and D^{(H)} following Eq. (87).

First, the RR are estimated from \hat{v}_{z} (Eq. (89)) by leveraging the 1-sparse property of a_{z}^{(R)} according to

$${𝒮}_{R}^{( z )} = {arg max}_{q = 1 , … , {Q}_{R}} \{ \vert {D}^{( R ) T} {\hat{v}}_{z} \vert \} , \qquad ( 91 )$$

where [symbol] _{R}^{(z)} denotes the respiration support of the z'th human, which selects the q'th frequency within {g_{q}^{(R)}}_{q=1}^{Q}^{_{R}} as the RR estimate \hat{f}_{R}^{(z)}. Next, the influence of respiration is subtracted by taking the residual vector \hat{v}′_{z}∈ [symbol] ^{L} as

$${\hat{v}}_{z}^{′} = {\hat{v}}_{z} - {d}_{{𝒮}_{R}}^{( z )} {\hat{a}}_{{𝒮}_{R}}^{( z )} , \qquad ( 92 )$$

where [symbol] ∈ [symbol] ^{L} is the atom of D^{(R)} corresponding to [symbol] _{R}^{(z)} and [symbol] = [symbol] \hat{v}_{z}/( [symbol] )∈ [symbol] is the estimated amplitude over [symbol] _{R}^{(z)}.

Then, the impact of interfering respiratory harmonics on HR estimation is mitigated by estimating D^{(R′,z)} and a_{z}^{(R′)}, followed by the removal of their contributions. To that sake, using Eq. (88) and \hat{f}_{R}^{(z)} (Eq. (91)), the respiration harmonics {g_{q}^{(R′,z)}}_{q=1}^{Q}^{_{R′,z}} and the complementary set {g_{q}^{(H′,z)}}_{q=1}^{Q}^{_{H′,z}} are estimated, by which D^{(R′,z)} and D^{(H′,z)} are assembled, respectively. Then the respiratory harmonics are subtracted from \hat{v}′_{z} (Eq. (92)) by

$${\hat{v}}_{z}^{″} = {\hat{v}}_{z}^{′} - D ( {R}^{′} , z ) {\hat{a}}_{z}^{( {R}^{′} )} \qquad ( 93 )$$

where â_{z}^{(R′)}=(D^{(R′,z)}^{^{T}} D^{(R′,z)}))⁻¹D^{(R′,z)}^{^{T}}\hat{v}′_{z}. Finally, the HR, f_{H}^{(z)} are estimated given \hat{v}″_{z}(∝) and D^{(H′,z)} by

$${𝒮}_{H}^{( z )} = {arg max}_{q = 1 , … , {Q}_{{H}^{′} , z}} \{ \vert {D}^{( {H}^{′} , z ) T} {\hat{v}}_{z}^{″} \vert \} , \qquad ( 94 )$$

where [symbol] _{H}^{(z)} denotes the heartbeat support of the z'th human, which points to the frequency within {g_{q}^{(H′,z)}}_{q=1}^{Q}^{_{H′,z}} that reflects the HR estimate, \hat{f}_{H}^{(z)}.

The continuous NCVSM framework of present disclosure enables leveraging the ongoing stream of estimates {\hat{f}_{H}^{(z)}, \hat{f}_{R}^{(z)}}_{z=1}^{Z} to manage random body motions (RBMs) and potential overlaps between HR and RR harmonics, aiming to enhance overall monitoring accuracy. Consequently, at each Tᵢₙₜ after predefined T_{ref} seconds, a three-stage signal refinement procedure is applied for the monitoring iteration, incorporating the current estimates along with prior ones as follows:
1. For the monitoring iteration at T_{ref}, all estimates are replaced with the median value derived from the samples collected up to that time, which can alleviate the fluctuations often observed at the onset of monitoring. For each Tᵢₙₜ following T_{ref}:
2. The vital estimates {\hat{f}_{H}^{(z)}, \hat{f}_{R}^{(z)}}_{z=1}^{Z} are replaced with the average of the estimations acquired in the last T_{avg}^{(H)} and T_{avg}^{(R)} seconds, respectively. This principle serves as an online filter that smooths the curve of estimates to ensure a gradual rate of changes in the vital signs.
3. The fixed bands of respiration and heartbeat, B^{(R)} and B^{(H)} respectively, are replaced with adaptive bands, centered around the vital estimates {\hat{f}_{H}^{(z)}, \hat{f}_{R}^{(z)}}_{z=1}^{Z} with small frequency margins. This adjustment focuses the frequency search within a limited range that more reliably tracks the subject's physiological state. Specifically, the adaptive bands of respiration and heartbeat are defined in [bpm] as B_{adp}^{(R)}(\hat{f}_{R}^{(z)})≙[\hat{f}_{R}^{(z)}−ε_{R}\hat{f}_{R}^{(z)}+ε_{R}] and B_{adp}^{(H)}(\hat{f}_{H}^{(z)})≙[\hat{f}_{H}^{(z)}−ε_{H}\hat{f}_{H}^{(z)}+ε_{H}], respectively, where ε_{R} and ε_{H} are predefined scalars which determine the margins of the corresponding bands.

Algorithm 2 below summarizes the E-VSDR approach for continuous NCVSM.

Algorithm 2: Continuous NCVSM by E-VSDR

Input: {\hat{v}_{z}}_{z=1}^{\hat{Z}}, B^{(R)}, B^{(H)}
for each z′th detected human z = 1, . . . , \hat{Z} :
1: Assemble D^{(R)} and D^{(H)} given B^{(R)} and B^{(H)} Eqs. (87), (90)
$2 : {𝒮}_{R}^{( z )} = {arg max}_{q = 1 , ... , {Q}_{R}} \{ \vert {D}^{( R ) T} {\hat{v}}_{z} \vert \} \text{=>} {\hat{f}}_{R}^{( z )} Eq . ( 91 )$
$3 : {d}_{{𝒮}_{R}}^{( z )} = {D}^{( R )} ( ∴ , {𝒮}_{R}^{( z )} ) and {\hat{a}}_{{𝒮}_{R}}^{( z )} = {d}_{{𝒮}_{R}}^{( z ) T} {\hat{v}}_{z} / ( {d}_{{𝒮}_{R}}^{( z ) T} {d}_{{𝒮}_{R}}^{( z )} )$
$4 : {\hat{v}}_{z}^{′} = {\hat{v}}_{z} - {d}_{{𝒮}_{R}}^{( z )} {\hat{a}}_{{𝒮}_{R}}^{( z )} Eq . ( 92 )$
5: Estimate D^{(R′, z)} and D^{(H′,z)} given Eqs. (88), (91)
$6 : {\hat{a}}_{z}^{( {R}^{′} )} = {( {D}^{( {R}^{′} , z ) T} {D}^{( {R}^{′} , z )} )}^{- 1} {D}^{( {R}^{′} , z ) T} {\hat{v}}_{z}^{′}$
7: \hat{v}_{z}″ = \hat{v}_{z}′ − D^{(R′, z)} â_{z}^{(R′)} Eq. (93)
$8 : {𝒮}_{H}^{( z )} = {arg max}_{q = 1 , … , {Q}_{{H}^{′} , z}} \{ \vert {D}^{( {H}^{′} , z ) T} {\hat{v}}_{z}^{″} \vert \} = > {\hat{f}}_{H}^{( z )} \qquad Eq . ( 94 )$
9: if current monitoring time t ≥ T_{ref} , then perform Signal Refinement (Subsection III-
$Ds ) and replace {B}^{( R )} = {B}_{adp}^{( R )} ( {\hat{f}}_{R}^{( z )} ) and {B}^{( H )} = {B}_{adp}^{( H )} ( {\hat{f}}_{H}^{( z )} )$
end for
$Output : Refined {\{ {\hat{f}}_{R}^{( z )} , {\hat{f}}_{H}^{( z )} \}}_{z = 1}^{Z}$

Algorithm 3 below outlines the complete approach of the present disclosure for robust multi-person localization and vital signs monitoring in low-SNR, cluttered environments using MIMO FMCW radar, based on the model described above.

Algorithm 3: Robust Multi-Person Localization and
Vital Signs Monitoring Using MIMO FMCW Radar

Input: T_{loc} , Tᵢₙₜ , T_{win} , {y[n, k, l]}, γ, L_{f}, Iₘₐₓ , B^{(R)}, B^{(H)}
At first T_{loc} do:
1: Assemble {Yₗ}ₗ₌₁^{L=T}^{_{locfs}}, A and B Eq. (56)
2: Filter {Yₗ}ₗ₌₁^{L} by (13) and recover{Xₗ}ₗ₌₁^{L} and [symbol] using RaLU-JSR
(Algorithm)
Output: [symbol] => \hat{Z} and {\hat{d}^{(z)}, \hat{θ}^{(z)}}_{z=1}^{\hat{Z}}
After T_{win} , for each Tᵢₙₜ do:
1: Assemble {Yₗ}ₗ₌₁^{L=T}^{_{win fs}} Eq. (56)
3: Use [symbol] to evaluate {\hat{x}_{S(z)}[l]}_{z=1}^{\hat{Z}} Eq. (84) and {\hat{v}_{z}}_{z=1}^{\hat{Z}} Eq. (47)
4: Estimate {f_{H}^{(z)}, f_{R}^{(z)}}_{z=1}^{Z} given {\hat{v}_{z}}_{z=1}^{\hat{Z}}, B^{(R)} and B^{(H)} using E-VSDR
(Algorithm 2)
Output: {\hat{f}_{H}^{(z)}, \hat{f}_{R}^{(z)}}_{z=1}^{\hat{Z}}

As mentioned above, the inventors designed a novel custom hardware phantom capable of simulating multi-person NCVSM in real-world, cluttered environments. The phantom utilizes recorded impedance signals, that capture variations in electrical impedance across the thorax caused by changes in thoracic volume. An example from a subject in [19] is shown in FIG. 25. These signals were selected to replicate the mechanical movements of the monitored individuals' thoraces via a dedicated vibration unit. The phantom is designed to support a wide range of cardio-respiratory patterns, including resting, rapid, and slow breathing or heartbeat, as well as pathological conditions such as apneas, arrhythmias, and Cheyne-Stokes respirations. By replicating realistic monitoring scenarios, including those required by healthcare providers, the phantom can facilitate rigorous validation of radar systems and algorithms for both single and multi-person NCVSM, ensuring their robustness and readiness for deployment in healthcare and IoT applications.

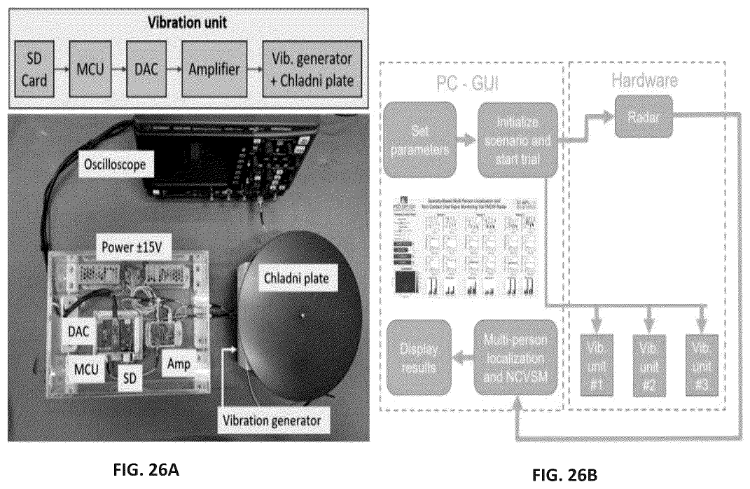

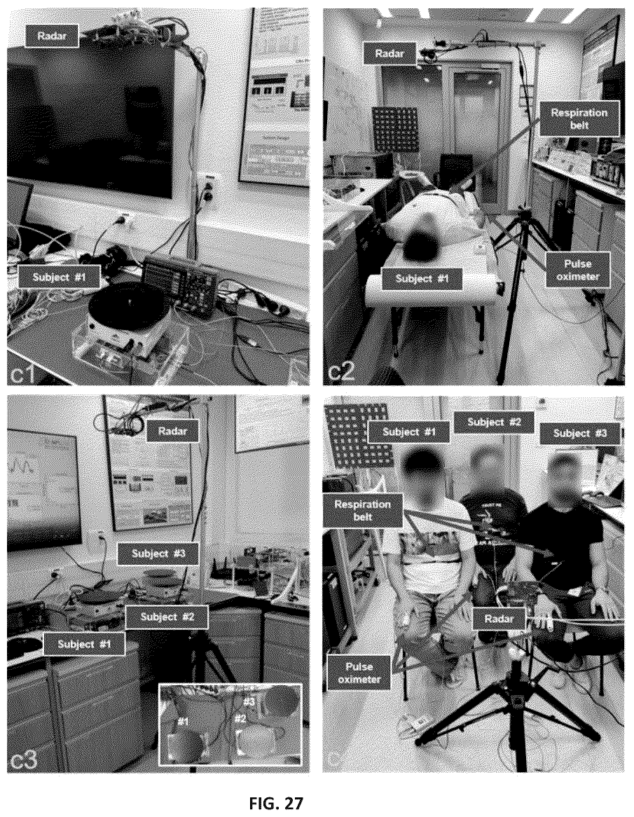

The phantom is designed in two configurations: one for single-person NCVSM, consisting of a single vibration unit (FIG. 27. c1), and another for multi-person NCVSM, comprising three independent vibration units (FIG. 27. c3). Each oscillating unit, as illustrated in FIG. 26A, includes the following key components: 1. Secure Digital card: Preloaded with recorded impedance signals, these cards store the digital data to be converted into mechanical oscillations. 2. Microcontroller unit (MCU), based on the Arduino processor: Reads the digital data from the SD card and sends it to 3. a digital-to-analog converter (DAC). 4. High-speed operational amplifier: A specialized amplifier adjusts the analog signal's current to effectively drive 5. a vibration generator fitted with a circular Chladni plate on top.

FIG. 26B depicts a block diagram of the validation process using the dedicated phantom in the multi-person setup. A graphical user interface (GUI) was developed under MATLAB environment to conveniently handle the validation stage using the hardware. This setup enables the validation of radar systems and algorithms across a wide range of scenarios, including the positioning of multiple individuals at varying distances and angles relative to the radar, and exhibiting different cardiopulmonary conditions.

In the following the performance of the suggested approach is evaluated and compared to existing techniques for both single person and multi-person NCVSM in a cluttered demonstration room, initially via the proposed custom phantom, then by human trials as elaborated below.

The proposed approach was validated by conducting 4 classes of experiments in a cluttered demonstration room containing multiple objects in the radar's FOV, such as computers, tables, and various electrical devices, as shown in FIG. 27. The classes were set as follows: c1—single-person phantom trials, c2—single-person human trials (lying back on a bed), c3—multi-person phantom trials, and c4—multi-person human trials (sitting on chairs). In each single-person setup (c1/c2), 9 simulated/human subjects were examined in 9 separate trials. In each multi-person setup (c3/c4), 9 simulated/human subjects were divided into 3 experiments of 3 people each. In every class, the targets were monitored continuously for 2 minutes with RR and HR estimates computed every Tᵢₙₜ=0.05 [s], using L frames of {Yₗ}ₗ₌₁^{L} (Eq. (56)) collected from the last T_{win}=30 [s], starting at T_{win}. The human trials were approved by the Weizmann Institutional Review Board-protocol #2214-1, and informed consent was obtained from 16 participants (9 males and 7 females, height from 156 to 183 [cm], weight from 47 to 87 [kg] and age from 19 to 54 years old).

In the phantom trials (c 1 and c 3), the simulated thoracic displacements were based on the impedance signal of 9 individuals from the resting scenario of [19], as exemplified in FIG. 25, that were converted into proper mechanical vibrations. The corresponding raw impedance signal and lead-2 ECG signal from [19], respectively served as the ground-truth (GT) references for comparing the RR and HR estimates [15], after down-sampling them to reach Tₛ. In the human trials (c2 and c4), the radar was aimed at the thorax of the subjects who were asked to breathe calmly and avoid large movements. As for the GT references of the human trials, the inventors used the g.Hiamp device (FDA-cleared and CE-certified medical product) for comparing the radar-based RR and HR estimates. Specifically, the GT-RR estimates were calculated from the torso circumference signal obtained from the Respiration Effort Sensor (respiration belt) whereas the GTHR estimates were calculated from the photoplethysmogram (PPG) signal obtained from the g.SpO2sensor (pulse oximeter).

The radar system selected for this work was Texas Instruments (TI) IWR1443BOOST 76 to 81 [GHz] mmWave Sensor Evaluation Module (EVM), connected to DCA1000EVM for data capture and streaming. The inventors employed the horizontal MIMO ULA setup of 2 transmitting antennas and 4 receiving antennas. In the single-person trials (c1 and c2), the SNR was improved by simultaneously using both transmitters to create a 1×K SIMO array with increased transmission power. In the general case of monitoring multiple targets (c3 and c4), the angular resolution was improved using a TDM transmission scheme that produced a virtual antenna array of size 1×\tilde{K} with \tilde{K}=2K, as depicted in FIG. 22 for K=4. The main radar parameters, reflected in the signal model in Eq. (56) are summarized in Table I. We note that for bandwidth B=ST_{c}≈4 [GHz], the range resolution is

$${d}_{res} = \frac{c}{2 B} \approx 3.75 [ cm ] .$$

In addition, the radar parameters listed in Table 7 through Eqs. (72), (79) and (80) with angle grid spacing set as Δ_{θ}=1, enable coverage over a radial distance range of dₘᵢₙ=4.29 [cm] to dₘₐₓ=4.24 [m] and an angular range of θₘᵢₙ=−90° to θₘₐₓ=+89°.

Multi-person NCVSM often necessitates both range and angular separation of targets. To evaluate these aspects, in the multi-person trials (c3 and c4), two of the three subjects were positioned at similar radial distances from the radar but at distinct azimuth angles, while the third subject was placed at a different radial distance and azimuth angle. The exact positionings for each class are provided in Table 8. For better readability, the outcomes of the multi-person trials (c3 and c4) are reported herein, while those of the single-person trials (c1 and c2) are provided in the supplementary material section below.

TABLE 7
MIMO FMCW radar parameters

| Parameter | Symbol | Value |
|---|---|---|
| Maximal chirp wavelength | λₘₐₓ | 3.9[mm] |
| Chirp duration | T_{C} | 57[μs] |
| ADC sampling rate | ƒ_{ADC} | 4[MHz] |
| Rate of frequency sweep | S | 70[MHz/μs] |
| Frame duration | T_{S} | 50[ms] |
| # of selected fast-time samples | N | 200 |
| # of chirps per frame | G | 40 |
| # of transmitters | J | 2 |
| # of receivers (virtual) | K(\tilde{K}) | 4(8) |

TABLE 8
Subject positionings for each class

| | Trial's | Distance | |
|---|---|---|---|
| Class | subject # | [m] | Angle [°] |
| c 1 | 1 | 0.7 | 0 |
| c 2 | 1 | 1.3 | 0 |
| c 3 | 1 | 0.80 | −10 |
| | 2 | 0.80 | +10 |
| | 3 | 0.85 | −15 |
| c 4 | 1 | 1.30 | −30 |
| | 2 | 1.30 | +30 |
| | 3 | 1.80 | 0 |

As discussed in the Introduction, it is necessary to achieve precise localization of all persons' thorax to accurately monitor their vital signs from the extracted thoracic vibrations. For every trial, the localization map was assessed given data from only the first five seconds of the monitoring session. That is, given {Yₗ}ₗ₌₁^{L} (Eq. (56)) assembled by the first L=fₛT_{loc} frames with T_{loc}=5 [s]. Here, the inventors compared the estimated 2D range angle map X̅ by the proposed RaLU-JSR (Algorithm 1) to that obtained using the Angle-FFT method used in [7], [26], the MUSIC approach [23], employed for each FFT range-bin, the second-order differential extension called SOD-MUSIC [29], the linear constrained minimum variance-based adaptive beamforming, here called LCMV [30], and the calibrated CIR technique with 5 past CIR maps to average in each frame, here called cal-CIR.

The parameters of RaLU-JSR were set as follows: The number of iterations and regularization parameter were selected to I=1000 and γ=100, respectively. Following the Lipschitz relation below Algorithm 1, the discretized matrices A and B lead to L_{f}=8.8439e+04 for the multi-person analysis when \tilde{K}=8 and L_{f}=6.3750e+04 for the single-person analysis when K=4. Finally, the vital frequencies of Π in Eq. (59) were drawn from the length L slow-time Nyquist grid determined by fₛ according to the respiration and heartbeat bands B^{(R)}=[0.1 0.5][Hz] and B^{(H)}=[0.83 1.67][Hz], respectively, corresponding to a normal resting state.

For a fair comparison, all maps were produced using identical range and angle grids, were normalized by the respective maximum value within a designated ROI of [0.5 2][m] and [−50+50][ °] and were slightly denoised by setting values below 0.05% of the maximum to zero. Here, S was determined by 2D selection of peaks that exceed a normalized power threshold of 0.1 and 0.4 for range and angle, respectively. In each localization figure, cyan circles (◯) indicate the true thoracic locations, while red crosses (X) represent the estimated positions based on the proposed detection scheme.

After the individuals are accurately located, their vital signs are monitored continuously for every Tᵢₙₜ, given [symbol] and the last L=fₛT_{win} frames of {Yₗ}ₗ₌₁^{L} (Eq. (56)) collected up to that time.

The inventors evaluated the performance of the E-VSDR method (Algorithm 2) for both single-person and multi-person NCVSM given {\hat{v}_{z}}_{z=1}^{\hat{Z}} (Eq. (85)) and the vital bands B^{(R)} and B^{(H)}. Recall that the last stage of the E-VSDR incorporates a signal refinement procedure designed to enhance accuracy in continuous NCVSM, by mitigating noise and possible overlaps between HR and RR harmonics. The parameters of the refinement were set to T_{ref}=5 [s], T_{avg}^{(H)}=3 [s], T_{avg}^{(R)}=5 [s] and ε_{H}=ε_{R}=5.

The results of the EVSDR were compared to those obtained using several state-of-the-art NCVSM techniques: 1. The method detailed in [8] for estimating RR and HR given the phase of a FMCW signal, called here PhaseReg. 2. FFT-based peak selection in each frequency band [10]-[12], termed here FFT. 3. The approach suggested in [29] which employs a FFT-based peak selection on the extracted phase after the removal of high order respiration harmonics via orthogonal projections, named here OrthProj. To assess the impact of the refinement on the results, the performance was also compared with these three techniques augmented with the same refinement procedure used in E-VSDR. They are referred to as 4. PhaseReg+, 5. FFT+ and 6. OrthProj+, respectively. As for the reference data, the GT-RR and GT-HR were calculated from the raw data of the contact sensors via the DFT spectrum [10], [10], [15], [9], [8], here padded to fit a 60-second time window to correspond to an optimal frequency resolution of 1 [bpm].

To compare the NCVSM methods fairly, it is assumed that all considered subjects were accurately detected and positioned. That is, the following comparison was performed given the same extracted thoracic vibrations {\hat{v}_{z}}_{z=1}^{\hat{Z}} (Eq. (85)) using the true locations of the examined subjects. In addition, all methods used the same vital frequency bands B^{(R)} and B^{(H)}, and all other settings that preceded this step were the same. To assess the accuracy of the estimation methods w.r.t. the references, the following evaluation metrics were used for each class: 1. Average empirical Cumulative Distribution Function (AeCDF), defined here as the average percentage of instances in which the estimate deviated from the reference output during a monitoring session (Y-axis) by less than a variable error threshold of bpm (X-axis), over all trials within the class. For example, given an error threshold of 2 [bpm], the corresponding value on the Y-axis represents the average proportion of the monitoring period in which the estimations fell within +−2 [bpm] relative to the references, also called success rate-2 [bpm]. 2. Root-Mean-Square Error (RMSE) analysis for each class, including subject-specific values along with the average and median of the specified class.

Since the phantom validations served as an intermediate step to determine the optimal parameters and configurations prior to conducting human trials, the description below begins by analyzing the results from the phantom trials (here multi-person c3). The analysis is conducted in three stages: localization, followed by NCVSM, and concluding with a performance evaluation.

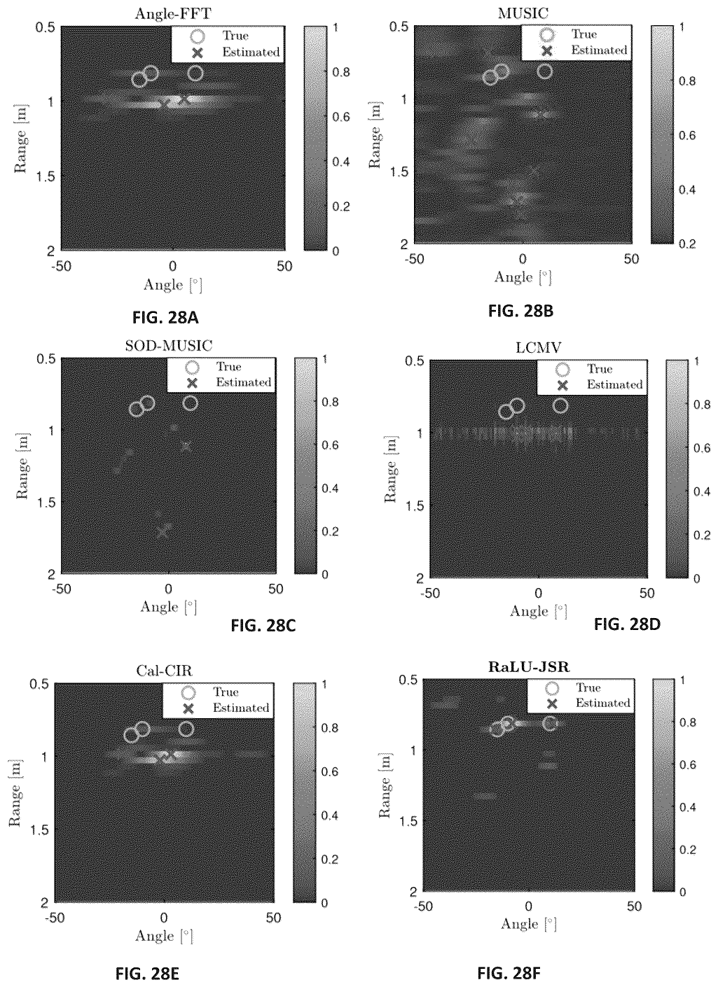

FIGS. 28A to 28F depict the compared localization maps of trial #2 from c3, normalized within the designated ROI. 1. Although the Angle-FFT (FIG. 28A) detected some reflections from the subjects, these were smeared due to resolution limitations and mispositioned, as the method was biased toward stronger reflections originating from the table supporting the plates. 2

The MUSIC approach (FIG. 28B) seeks highly reflecting or oscillating objects in each range-bin. Hence, in the cluttered scenario, it mistakenly highlighted clutter over humans, which deteriorated performance in both detection and positioning. 3. The SOD-MUSIC (FIG. 28C) map was considerably cleaner than its predecessor. However, the influence of clutter remained substantial, hindering the correct localization of the subjects. 4. For the LCMV map (FIG. 28D), the table completely masked the presence of the targets. 5. The cal-CIR map (FIG. 28E) resembled that of Angle-FFT in its ability to identify certain reflections from the phantom. However, these reflections were far weaker than those from the table, leading to inaccurate localization. 6. In contrast to the compared localization techniques, the proposed

RaLU-JSR (FIG. 28F) detected the right number of simulated subjects and their location, without range error and with angular error of less than 3 [°], far below the theoretical resolution limitation of ≈15 [°] when using 8 receivers. These results stem from the fact that RaLU-JSR uniquely leverages both human vital frequencies and the sparse properties of the data through the proposed bilinear model in Eq. (56).

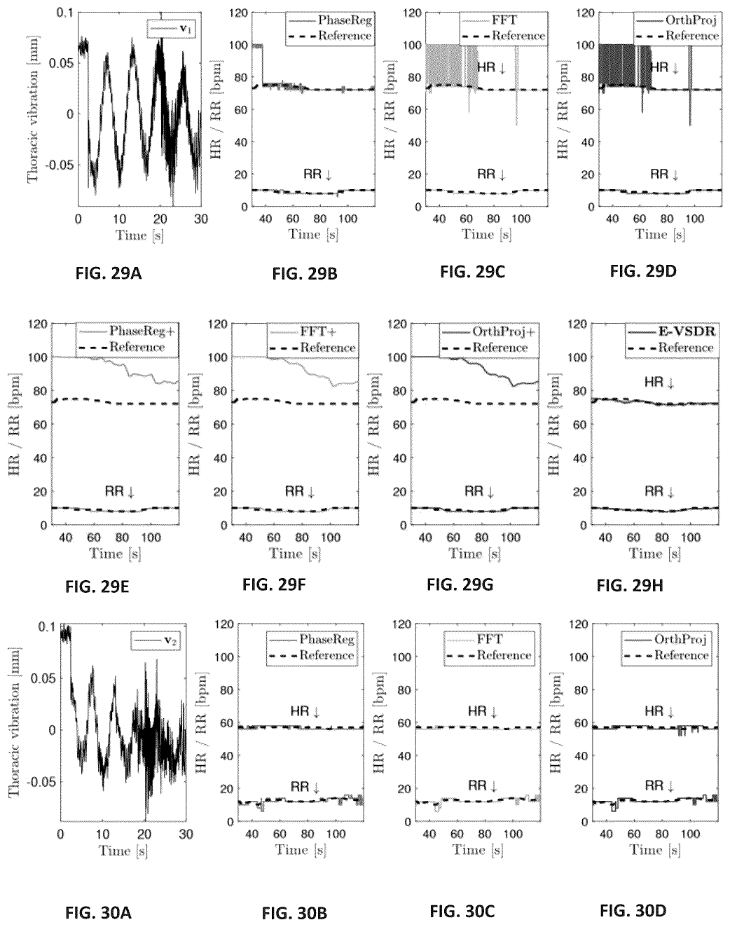

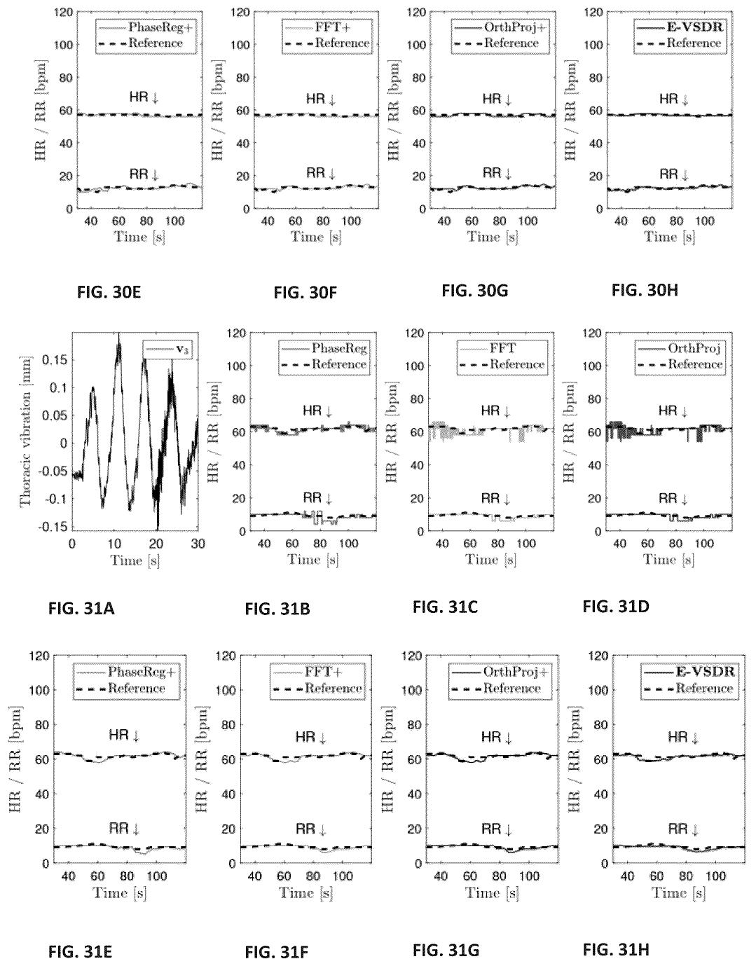

FIGS. 29A to 29H, 30A to 30H, and 31A to 31H depict the NCVSM outcomes of this trial. The results were produced by all 7 compared methods relative to GT references, given the corresponding vibrations {\hat{v}_{z}}_{z=1}^{\hat{Z}=3}. The inventors observed several key findings. First, since the resting heartbeat typically spans a relatively wide frequency band (here, 0.83 to 1.67 [Hz]), this can lead to interference from RR harmonics competing with the true HR during the frequency search. This is evident in the case of \hat{v}₁ (FIGS. 29A to 29H), where both the original compared methods (PhaseReg (FIG. 29B), FFT (FIG. 29C), and OrthProj (FIG. 29D)) and their refined versions (PhaseReg+ (FIG. 29E), FFT+ (FIG. 29F), and OrthProj+ (FIG. 29G)) were significantly affected by the large variability in HR estimations. The considerable fluctuation is particularly detrimental to the refined variants, as they constrict the frequency search area based on the median outcome of the onset measurements, which deviated significantly from the true values. Additionally, for \hat{v}₂ (FIGS. 30A to 30G) and \hat{v}₃ (FIGS. 31A to 31G), the estimation curves of the refined methods (FIGS. 30E to 30G, and 31E to 31G) exhibited patterns with reduced noise and more closely matched the reference curves compared to their original versions (FIGS. 30B to 30D, and 31B to 31D). However, only the HR and RR estimates by the proposed E-VSDR demonstrated a high degree of similarity to the reference values across all three subjects.

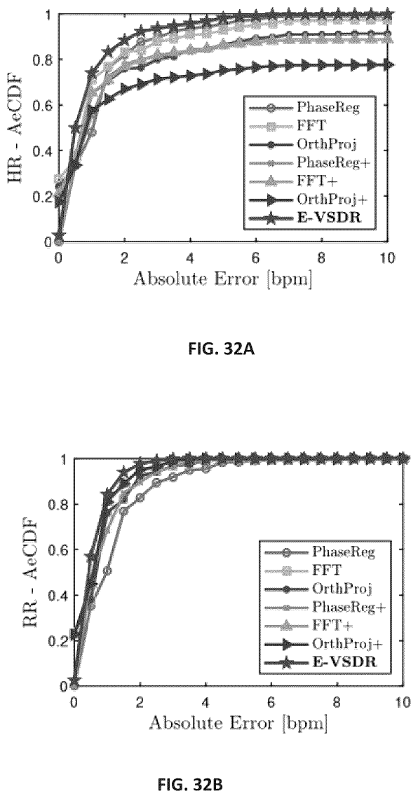

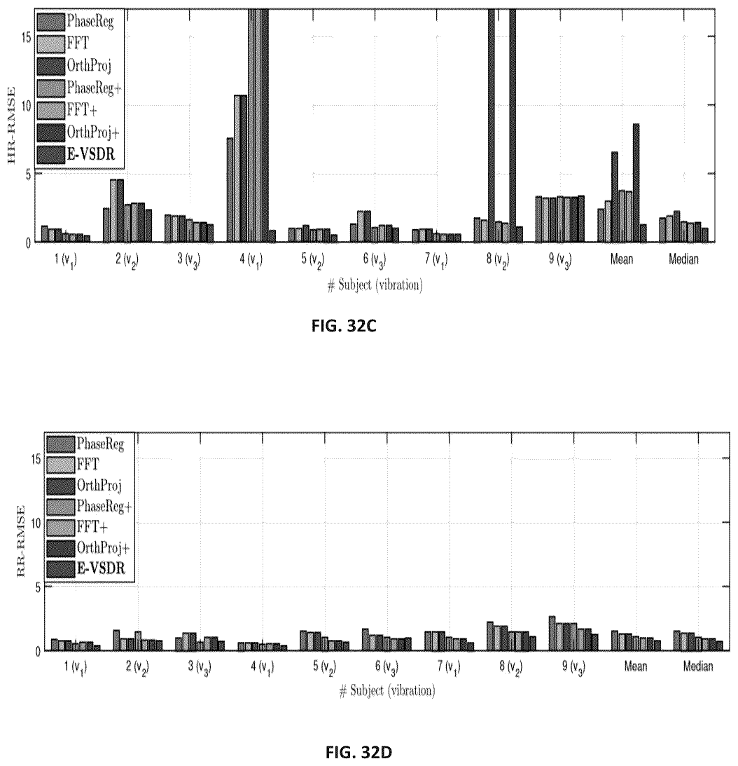

FIGS. 32A and 32B show the 9 subjects-AcCDF of class c3. The AcCDF was calculated for both HR and RR estimations by all examined methods, as a function of absolute error thresholds ranging from 0 to 10 [bpm]. First, one sees that the EVSDR outperforms all other compared methods for every error threshold greater than 0.5 [bpm]. For instance, an average success rate of 2 (ASR2), 3 (ASR3) and 4 (ASR4) [bpm] was exclusively reached by our E-VSDR method for accuracy of 88.41%, 93.66% and 95.74%, respectively, for HR estimation and accuracy of 97.74%, 99.77% and 100%, respectively, for RR estimation. For the numerical values obtained by all compared methods, see Table 9. Interestingly, the refinements applied to the competing methods primarily improved the RR performance, although they still did not surpass the EVSDR. While the E-VSDR demonstrated the best overall performance in the empirical CDF analysis, the relatively small performance gap, particularly in RR estimations, can be attributed to the phantom mechanism's reliance on impedance signals with prominent cardiopulmonary information, which facilitates adequate estimation performance across all methods.

Finally, FIGS. 32C and 32D present the HR-RMSE and RR-RMSE distributions, respectively, for each NCVSM method across the 9 subjects of class c 3, along with the corresponding average and median values. The proposed E-VSDR achieved the lowest average and median RMSE values for both HR and RR estimations, even when using the outlier-tolerant median metric. Specifically, the class average RMSE (ARMSE) was as low as 1.23 and 0.73 for HR and RR estimation, respectively, with the values of the compared techniques shown in Table 9. Additionally, E-VSDR obtained superior RMSE scores for most subjects, regardless of their location relative to the radar, with only minor deviations observed for the remaining subjects. In terms of median RMSE, the refinements applied to PhaseReg, FFT, and OrthProj enhanced performance for both HR and RR estimations; however, they did not outperform the E-VSDR, which underscores the strength of the core harmonics-resilient, dictionary-based approach of the E-VSDR. Both the localization and NCVSM results via the phantom trials instill confidence to advance to the human trials with the finalized algorithm and parameters as well as proper trial configurations.

TABLE 9
Average success rate [%] for 2 (ASR2), 3 (ASR3)
and 4 (ASR4) [bpm] as well as average root-mean-
squarederror (ARMSE) for HR and RR estimations by the
compared NCVSM methods, for multi-person classes c3 and c4.

| Class | Rate | Method | ASR2 | ASR3 | ASR4 | ARMSE |
|---|---|---|---|---|---|---|
| c3 | HR | PhaseReg | 82.02 | 89.48 | 93.66 | 2.34 |
| | | FFT | 83.25 | 87.83 | 90.85 | 2.96 |
| | | OrthProj | 75.85 | 80.00 | 84.30 | 6.50 |
| | | PhaseReg+ | 77.88 | 81.14 | 83.18 | 3.71 |
| | | FFT+ | 76.91 | 81.99 | 83.94 | 3.65 |
| | | OrthProj+ | 67.04 | 71.33 | 72.83 | 8.53 |
| | | E-VSDR | 88.41 | 93.66 | 95.74 | 1.23 |
| | RR | PhaseReg | 82.81 | 91.80 | 95.54 | 1.47 |
| | | FFT | 92.27 | 96.87 | 99.20 | 1.27 |
| | | OrthProj | 92.27 | 96.87 | 99.20 | 1.27 |
| | | PhaseReg+ | 89.57 | 96.65 | 99.24 | 1.06 |
| | | FFT+ | 95.20 | 99 | 100 | 0.95 |
| | | OrthProj+ | 95.20 | 99 | 100 | 0.95 |
| | | E-VSDR | 97.74 | 99.77 | 100 | 0.73 |
| c4 | HR | PhaseReg | 61.77 | 72.55 | 77.91 | 7.43 |
| | | FFT | 57.91 | 63.30 | 66.87 | 10.03 |
| | | OrthProj | 57.12 | 62.48 | 66.04 | 10.11 |
| | | PhaseReg+ | 71.58 | 75.30 | 80.23 | 4.01 |
| | | FFT+ | 70.01 | 79.84 | 83.26 | 5.20 |
| | | OrthProj+ | 70.60 | 80.60 | 84.53 | 5.02 |
| | | E-VSDR | 87.10 | 94.12 | 95.54 | 1.33 |
| | RR | PhaseReg | 68.88 | 79.51 | 88.33 | 2.46 |
| | | FFT | 81.01 | 88.99 | 92.01 | 2.02 |
| | | OrthProj | 81.01 | 88.99 | 92.01 | 2.02 |
| | | PhaseReg+ | 78.36 | 87.46 | 92.53 | 1.95 |
| | | FFT+ | 86.66 | 93.19 | 95.87 | 1.51 |
| | | OrthProj+ | 86.66 | 93.19 | 95.87 | 1.51 |
| | | E-VSDR | 94.14 | 98.12 | 98.69 | 0.98 |

After drawing significant conclusions from the phantom trials, the inventors proceeded with the human trials using similar parameters and configurations. The corresponding localization and NCVSM results for class c4 (multi-person human trials) are presented below.

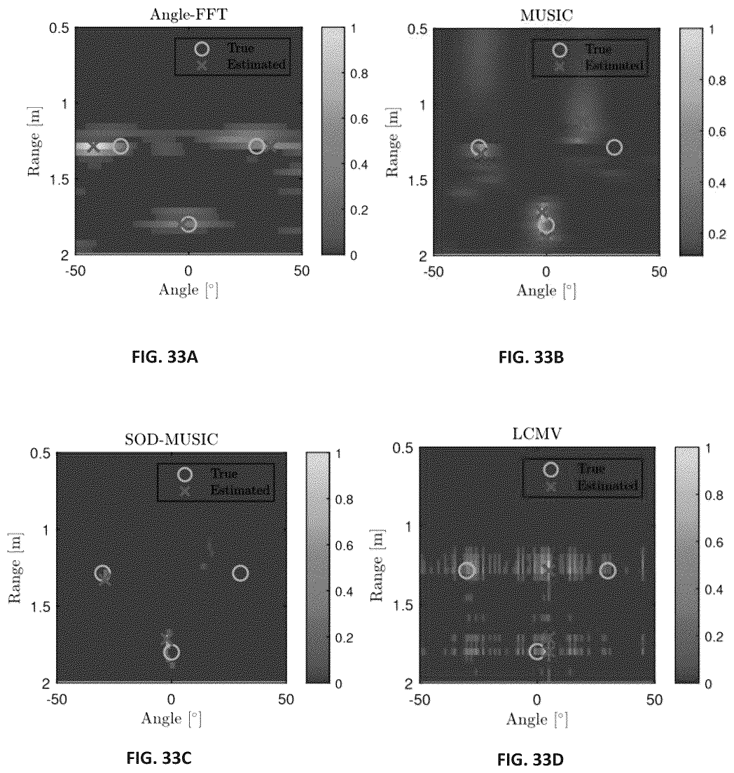

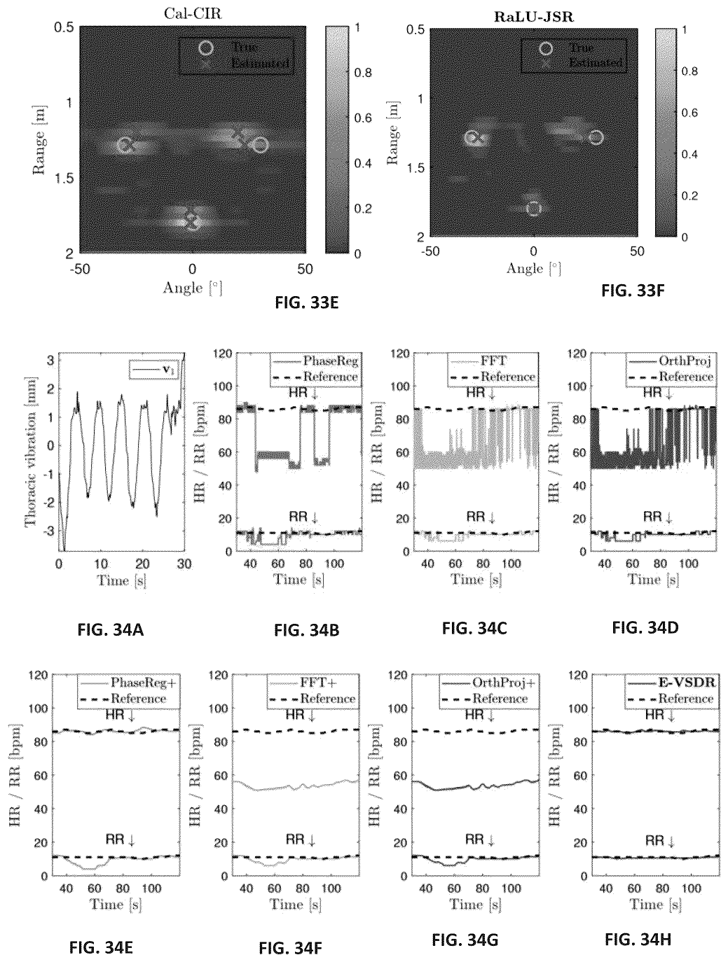

FIGS. 33A to 33F below depict the compared normalized localization maps within the designated ROI of class c4-trial #3. One can observe the following: 1. While the Angle-FFT (FIG. 33A) successfully detected the presence of all three individuals, it exhibited coarse angular errors, particularly for the equidistant subjects at 1.30 [m]. 2. Although MUSIC (FIG. 33B) managed to locate two out of the three subjects, its high sensitivity caused clutter artifacts to emerge, which led to the miss detection of the third one. 3. The enhanced SOD-MUSIC algorithm (FIG. 33C) improved upon MUSIC by sharpening the output map and mitigating clutter. However, it also failed to detect one of the subjects. 4. For the LCMV (FIG. 33D), while the distance estimates were reasonably accurate, the method generated too many angular candidates, causing the solution to converge to the center and thus fail to resolve the equidistant individuals at 1.30 [m]. 5. The cal-CIR map (FIG. 33E) provided an accurate depiction of the subjects' locations in terms of both distance and angle. However, its limited ability to differentiate between clutter and humans resulted in the selection of non-human objects alongside the actual subjects. 6. In contrast, only the suggested RaLU-JSR (FIG. 33F) detected precisely all 3 humans and identified the position of their thorax, with an angular error of less than 5[°] for each subject, demonstrating distinguished performance in both detection and positioning.

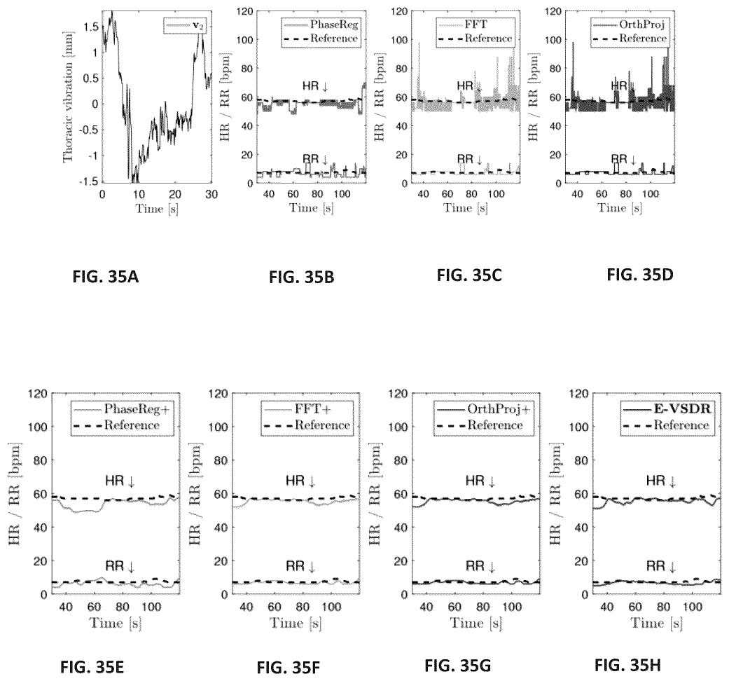

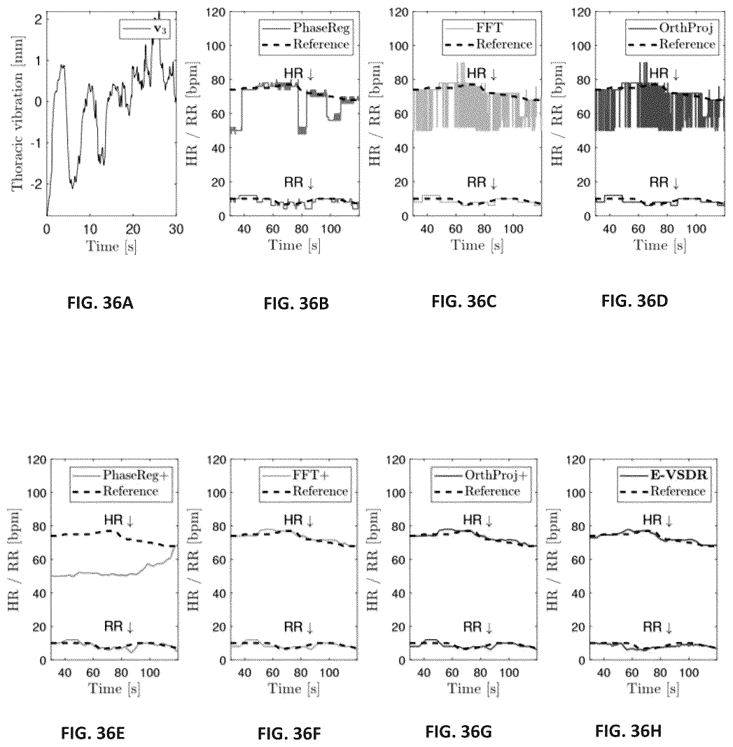

FIGS. 34A to 34H, 35A to 35H, and 36A to 36H, present the NCVSM outcomes of this trial. One can notice significant variability in HR estimates among the compared methods (PhaseReg (FIGS. 34B, 35B, and 36B), FFT (FIGS. 34C, 35C, and 36C), and OrthProj (FIGS. 34D, 35D, and 36D)) across all three subjects. This variability resulted in persistent drifts in the refined versions: FFT+ (FIG. 34F) and OrthProj+ (FIG. 34G) for \hat{v}₁) and PhaseReg+ for \hat{v}₂ and \hat{v}₃ ((FIGS. 35E, 36E). In contrast, the proposed E-VSDR demonstrated remarkable robustness, effectively handling the challenging noise introduced by low SNR, multipath effects, possible RBMs, clutter, and interfering harmonics, for all three subjects.

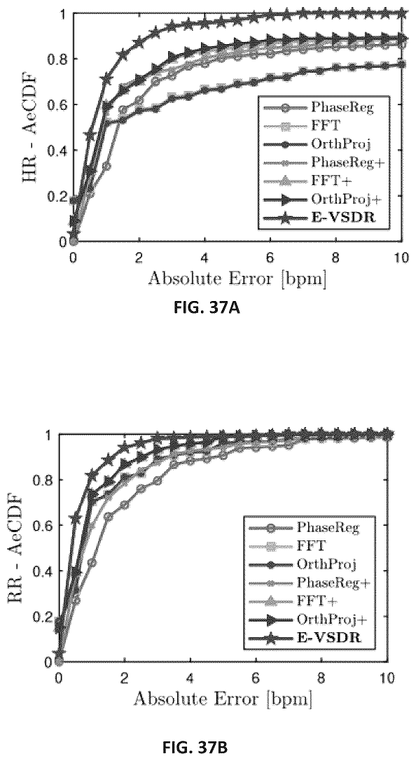

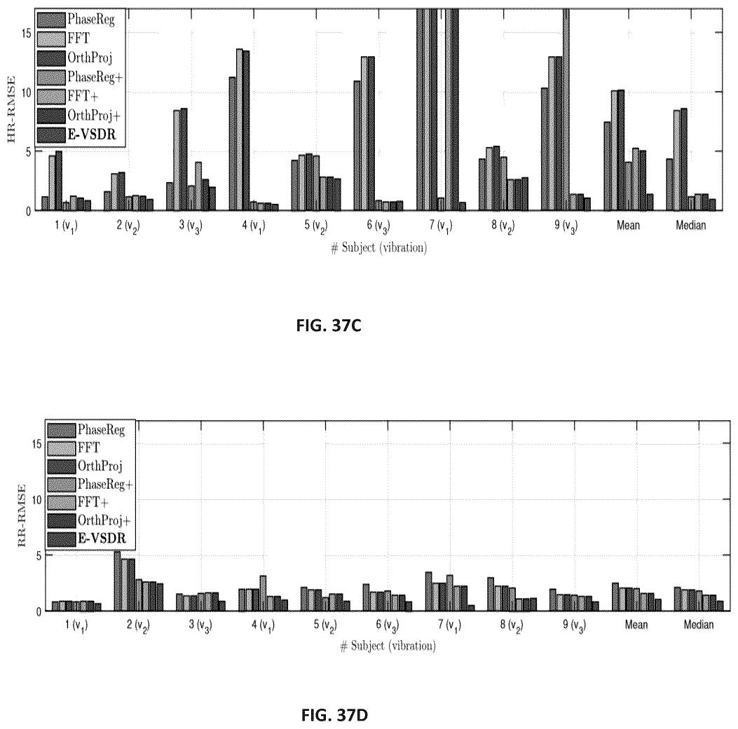

FIGS. 37A and 37B show the 9 subjects-AcCDF of class c4. As in the phantom case, the E-VSDR consistently outperformed all other methods for thresholds above 0.5 [bpm], achieving ASR2, ASR3 and ASR4 accuracies of 87.10%, 94.12% and 95.54%, respectively, for HR estimation and 94.14%, 98.12% and 98.69%, respectively, for RR estimation. Detailed values for the compared methods are provided in Table III. One can notice a considerable difference in performance compared to the other techniques, especially in the more challenging task of HR monitoring due to the weak heartbeat signature, in favor of the proposed approach. Another noteworthy observation is that in contrast to the phantom to trials c3, the refinements applied to the competing methods (PhaseReg+, FFT+ and OrthProj+) yielded performance improvements for both HR and RR estimations. However, these enhancements remained insufficient to surpass the performance of the proposed approach.

Finally, FIGS. 37C and 37D illustrate the HR-RMSE and RR-RMSE distributions, respectively, for each NCVSM method in class c4. Similar to the phantom trials c3, the proposed E-VSDR achieved the lowest average and median RMSE values for both HR and RR estimations. Specifically, the class ARMSE was as low as 1.33 and 0.98 for HR and RR estimations, respectively, with the values for the compared methods presented in Table 9. Furthermore, the refinements applied in PhaseReg+, FFT+, and OrthProj+ resulted in improved performance for both HR and RR estimations, as observed for both the average and median metrics. However, even with these enhancements, these methods did not outperform the RMSE scores of the E-VSDR.

The obtained results underline the robustness of the EVSDR method, which uniquely integrates a dictionary-based recovery with prior knowledge of cardiopulmonary activity to accurately estimate heartbeat and respiratory rates, even in the presence of considerable noise and interfering harmonics, in various experimental setups.

In the following, the inventors provide the results of the single-person phantom trials c1 and the single-person human trials c2 are described to support and strengthen the conclusions of the present disclosure.

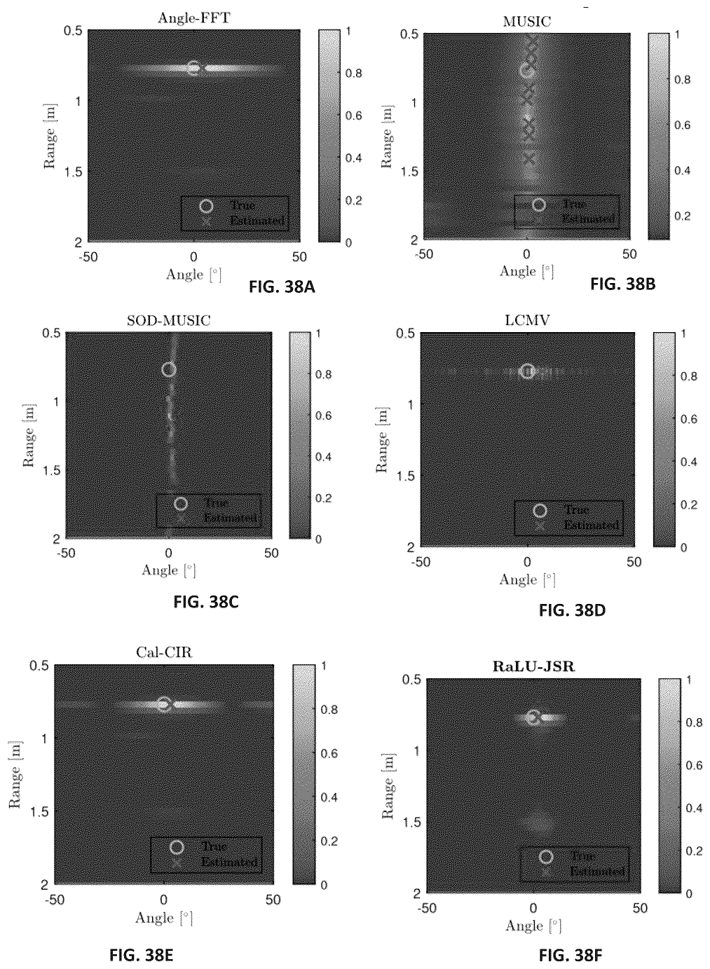

FIGS. 38A to 38F present the normalized localization maps within the designated ROI for trial #2 of class c1. Although the Angle-FFT method (FIG. 38A) exhibited a wide smearing effect, this did not hinder the detection of the subject's thorax in this single-person scenario, with only a minor angular error of 4[°]. Both MUSIC (FIG. 38B) and SOD-MUSIC (FIG. 38C), applied on each range-bin, incorrectly highlighted clutter in addition to the human target, resulting in inaccurate detection and positioning. While increasing the detection threshold may address this issue for single-person scenarios, it would significantly limit localization performance in multi-person cases. The LCMV

(FIG. 38D) map achieved an acceptable localization result, though it introduced an angular error of 7 [°]. The cal-CIR map (FIG. 38E) successfully detected and positioned the subject with an angular error of only 3 [°], albeit with the presence of minor side lobes and smearing effects. Lastly, the proposed RaLU-JSR (FIG. 38F) method generated a clean map with sharp detection lobe, achieving the smallest angular error of 2 [°]. Moreover, the narrow lobe width highlights the method's potential for accurate multitarget localization, as shown in Section V of the main paper.

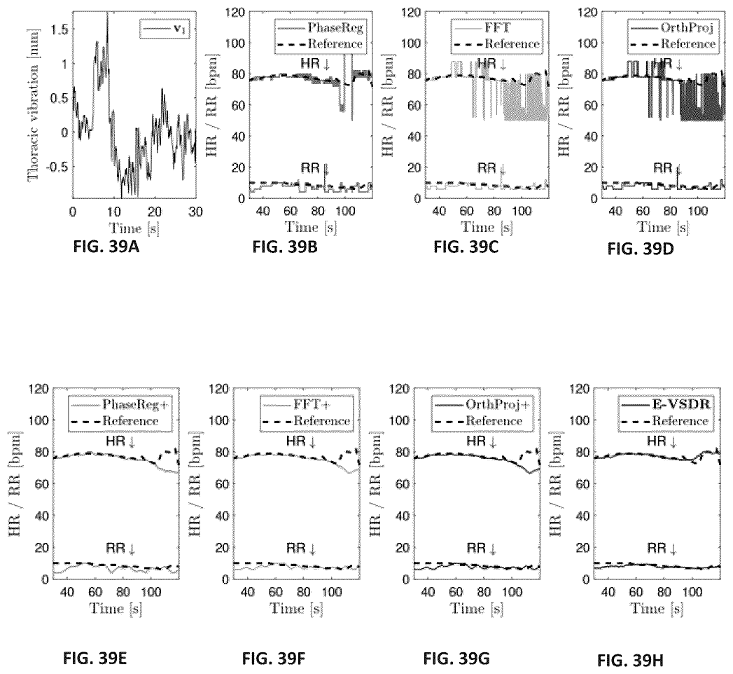

FIGS. 39A to 39H illustrate the NCVSM outcomes for this trial, generated by all 7 compared methods relative to the GT references, given the extracted \hat{v}₁. In this example, the estimated curves generally align with their respective reference curves across all methods, as expected given the relative simplicity of monitoring a single simulated subject via the custom phantom. Notably, while the refinement procedures (PhaseReg+ (FIG. 39E), FFT+ (FIG. 39F), and OrthProj+ (FIG. 39G)) improved the smoothness of the estimated curves and consequently enhanced performance, significant deviations from the reference curves were still observed at multiple time points for both HR and RR. Among the compared methods, the proposed E-VSDR (FIG. 39H) exhibited the closest resemblance to the reference curves throughout the monitoring duration.

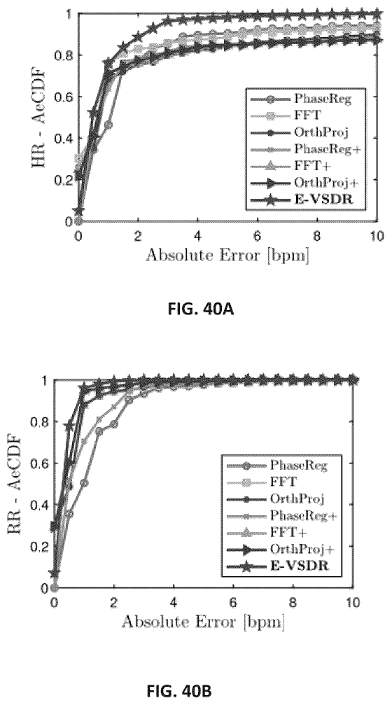

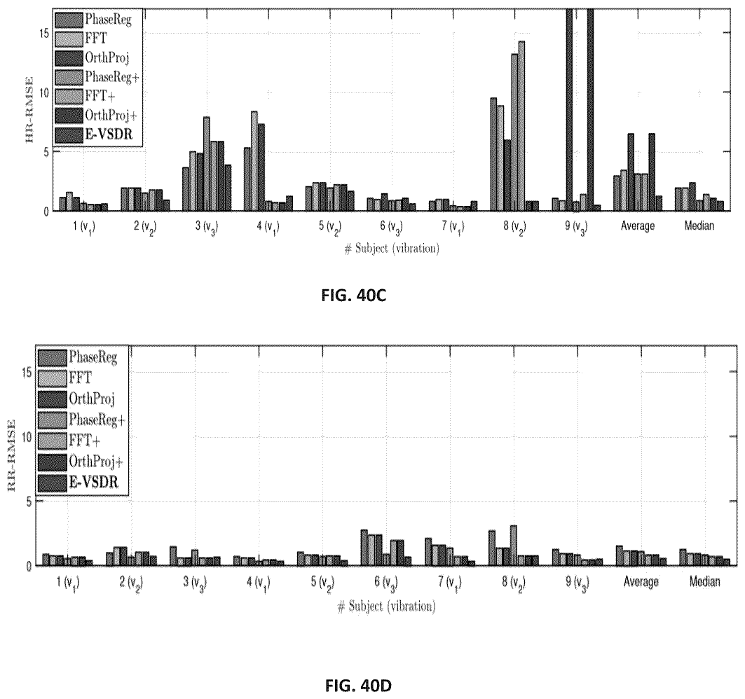

FIGS. 40A and 40B show the HR-AcCDF (FIG. 40A) and RR-AcCDF (FIG. 40B) of class c1. First, one sees that the E-VSDR outperforms all other compared methods for every error threshold greater than 0.5 [bpm].

Specifically, it achieved an ASR2, ASR3, and ASR4 of 88.61%, 96.21%, and 97.33%, respectively, for HR estimation, and 99.38%, 100%, and 100%, respectively, for RR estimation. The values of the compared methods are given in Table 10.

TABLE 10

| Class | Rate | Method | ASR2 | ASR3 | ASR4 | ARMSE |
|---|---|---|---|---|---|---|
| c1 | HR | PhaseReg | 77.29 | 85.26 | 89.63 | 2.91 |
| | | FFT | 82.99 | 85.91 | 87.48 | 3.39 |
| | | OrthProj | 75.56 | 79.83 | 82.41 | 6.44 |
| | | PhaseReg+ | 78.48 | 81.54 | 84.35 | 3.08 |
| | | FFT+ | 76.03 | 80.33 | 83.83 | 3.08 |
| | | OrthProj+ | 77.20 | 81.01 | 83.83 | 6.45 |
| | | E-VSDR | 88.61 | 96.21 | 97.33 | 1.18 |
| | RR | PhaseReg | 78.69 | 93.44 | 96.64 | 1.50 |
| | | FFT | 94.45 | 96.40 | 98.19 | 1.12 |
| | | OrthProj | 94.45 | 96.40 | 98.19 | 1.12 |
| | | PhaseReg+ | 87.11 | 96.99 | 97.83 | 1.03 |
| | | FFT+ | 96.85 | 98.63 | 99.08 | 0.78 |
| | | OrthProj+ | 96.85 | 98.63 | 99.08 | 0.78 |
| | | E-VSDR | 99.38 | 100 | 100 | 0.49 |
| c2 | HR | PhaseReg | 56.74 | 70.96 | 77.24 | 4.79 |
| | | FFT | 60.42 | 67.18 | 70.54 | 6.20 |
| | | OrthProj | 58.04 | 65.04 | 68.91 | 6.50 |
| | | PhaseReg+ | 70.99 | 77.62 | 83.22 | 3.48 |
| | | FFT+ | 83.42 | 89.15 | 92.15 | 2.13 |
| | | OrthProj+ | 80.04 | 87.36 | 90.81 | 2.27 |
| | | E-VSDR | 85.93 | 93.43 | 96.91 | 1.45 |
| | RR | PhaseReg | 81.54 | 89.63 | 94.08 | 2.21 |
| | | FFT | 92.27 | 94.80 | 96.44 | 1.80 |
| | | OrthProj | 92.27 | 94.80 | 96.44 | 1.80 |
| | | PhaseReg+ | 91.18 | 96.59 | 98.10 | 1.00 |
| | | FFT+ | 96.37 | 98.41 | 100 | 0.74 |
| | | OrthProj+ | 96.37 | 98.41 | 100 | 0.74 |
| | | E-VSDR | 97.00 | 100 | 100 | 0.55 |

Interestingly, the refinements applied to the competing methods yielded improvements primarily in RR estimation performance, yet they remained inferior to the E-VSDR. While the E-VSDR exhibited superior AcCDF performance, the relatively narrow performance gap in RR estimation can be attributed to the experimental setup. The single-person phantom trial generates distinct thoracic displacements directly in front of the radar antennas, enabling all methods to achieve reasonably accurate RR estimations.

FIGS. 40C and 40D present the HR-RMSE and RR-RMSE distributions, respectively, for each NCVSM method across the 9 subjects of class c1, along with the corresponding average and median values. The proposed E-VSDR outperformed all other methods, achieving the lowest average and median RMSE for both HR and RR estimations, even when using the outlier-tolerant median metric. Specifically, the class ARMSE was as low as 1.18 and 0.49 for HR and RR estimation, respectively, with the values of the compared techniques given in Table 10. Additionally, the E-VSDR obtained superior RMSE scores for most subjects, with only minor deviations observed for the remaining subjects. Observing the median RMSE, although refinements applied to PhaseReg, FFT, and OrthProj improved HR and RR estimation results, these enhancements remained insufficient to outperform the E-VSDR.

After drawing meaningful conclusions from the phantom trials, the inventors proceeded with the human trials using similar parameters and configurations. The corresponding localization and NCVSM results for class c2 (single-person human trials) are presented below.

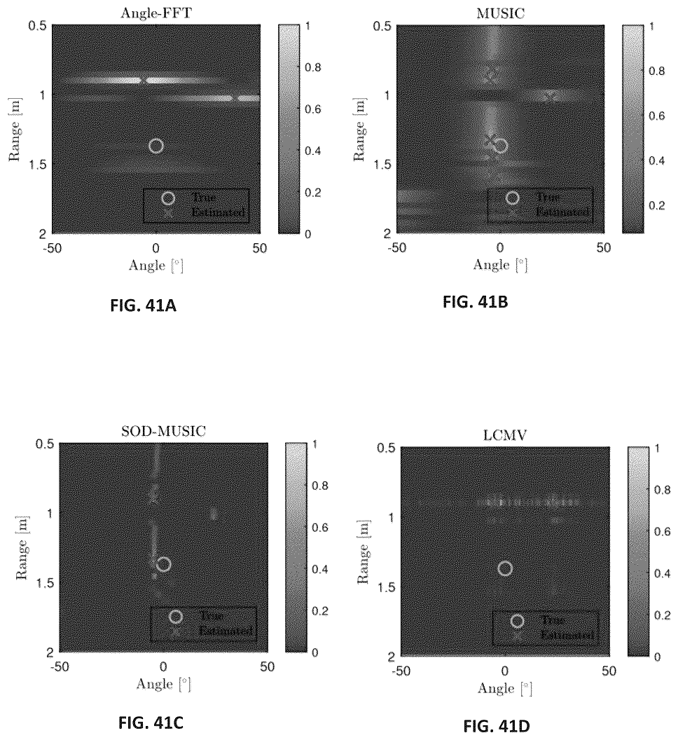

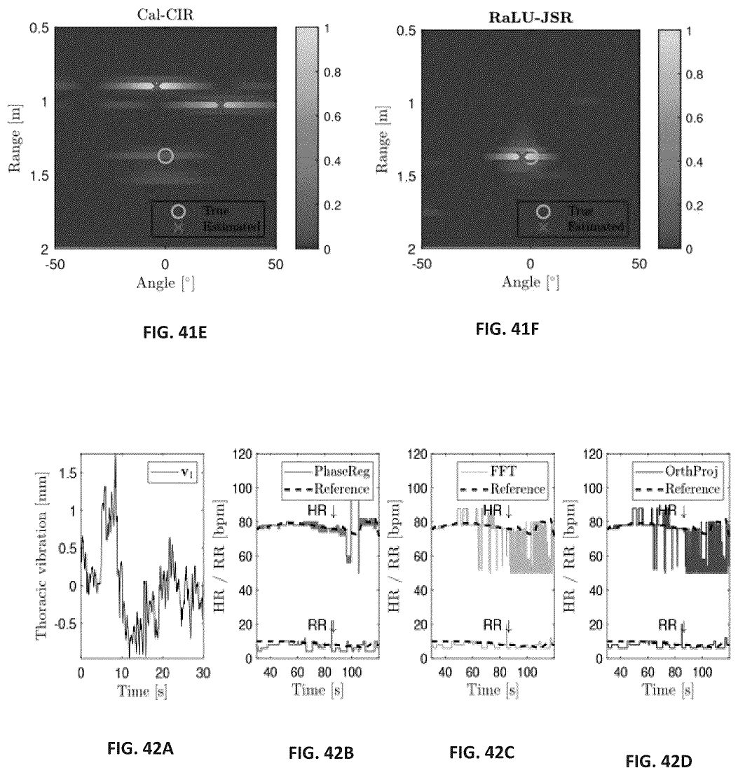

FIGS. 41A to 41F depict the compared normalized localization maps for trial #7 of class c2, within the designated ROI. One can observe the following: 1. The Angle-FFT method (FIG. 41A) incorrectly identified highly reflective clutter (at 0.9 and 1.02 [m]) located before the human target (at 1.37 [m]), with minimal power concentrated at the true position. 2. MUSIC algorithm (FIG. 41B) detected returns near the true location but lacked the accuracy to precisely pinpoint the individual. 3. The enhanced SOD-MUSIC (FIG. 41C) improved upon MUSIC by refining the output map and reducing clutter interference. However, it still failed to accurately position the detected subject. 4. The LCMV method (FIG. 41D) focused on clutter at 0.9 [m], similar to AngleFFT, leading to a misidentified location. 5. The cal-CIR map (FIG. 41E) revealed reflections from both clutter (at 0.9 [m] and 1.02 [m]), and from the true subject location (at 1.37 [m]). However, its inability to effectively differentiate between clutter and human reflections resulted in incorrect selection of the clutter. 6. In contrast, the proposed RaLU-JSR

(FIG. 41F) precisely detected a single subject and accurately identified the position of its thorax, with an angular error of 4[°], showcasing superior performance in both detection and positioning.

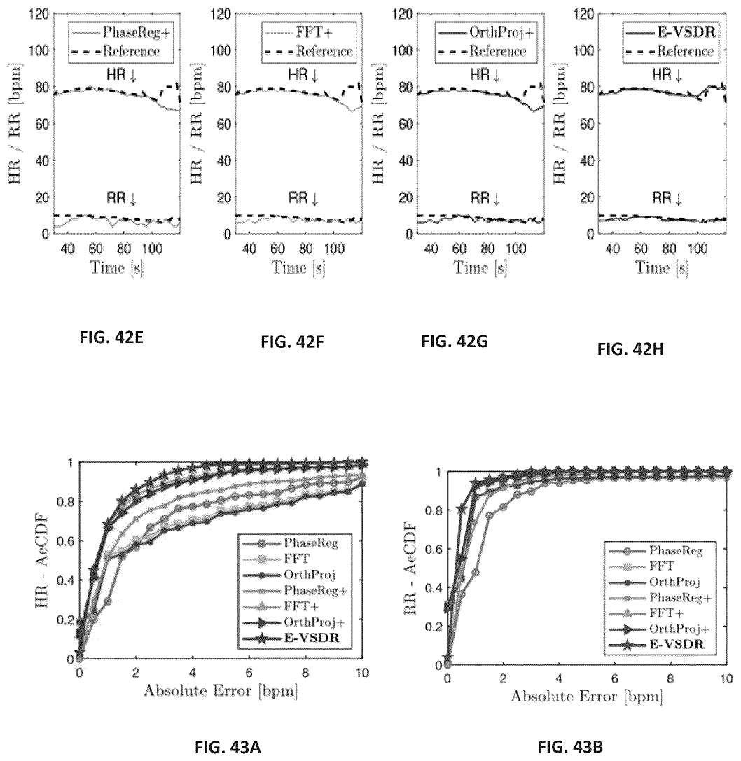

FIGS. 42A to 42H illustrates the NCVSM results for this trial. Significant variability in HR estimates can be observed among the original compared methods (PhaseReg (FIG. 42B), FFT (FIG. 42C), and OrthProj (FIG. 42D)), particularly in the later stages of the monitoring where a rapid change in HR was observed. This variability deteriorated the ability to reliably monitor the HR, even with the refinements applied (FFT+ (FIG. 42F), OrthProj+ (FIG. 42G), and PhaseReg+ (FIG. 42E)). The E-VSDR (FIG. 42H) not only produced an accurate RR estimates curve but also demonstrated exceptional robustness to challenging noise caused by interfering harmonics and rapid physiological state changes, effectively capturing the rapid HR fluctuations at the end of the monitoring period.

FIGS. 43A and 43B present the AeCDF results for HR (FIG. 43A) and RR

(FIG. 43B) estimation across the 9 subjects in class c2. Consistent with the phantom trials, the E-VSDR outperformed all other methods for thresholds above 0.5 [bpm], achieving ASR2, ASR3, and ASR4 accuracies of 85.93%, 93.43%, and 96.91%, respectively, for HR estimation, and 97.00%, 100%, and 100%, respectively, for RR estimation. Detailed values for the compared methods are provided in Table 10. While the refinements applied to the competing methods (PhaseReg+, FFT+, and OrthProj+) improved both HR and RR estimations compared to their unrefined counterparts, these enhancements were still insufficient to surpass the performance of the E-VSDR, even in the single-person trials where the radar was directly aimed at the subject's thorax.

Finally, FIGS. 43C and 43D illustrate the HR-RMSE and RR-RMSE distributions for each NCVSM method across the 9 subjects in class c2. Similar to the phantom trials, the proposed E-VSDR achieved the lowest average and median RMSE values for both HR and RR estimations. Specifically, the class ARMSE was as low as 1.45 and 0.55 for HR and RR estimations, respectively, with the values for the compared methods presented in Table 10. Furthermore, the refinements applied in PhaseReg+, FFT+, and OrthProj+ resulted in improved performance for both HR and RR estimations, as observed for both the average and median metrics. However, even with these enhancements, these methods did not outperform the RMSE scores of the E-VSDR.

1. A monitoring system for use in monitoring vital signs of one or more subjects, the monitoring system comprising a control system configured for signal communication with a frequency modulated continuous wave (FMCW) radar to process measured data, which is received from a single-channel front end of each of at least one receiver of said FMCW radar and which is in the form of data matrix indicative of consecutive beat signals, and provide output data indicative of vital signs of the subjects in a region of interest (ROI), said control system comprising a data processing utility comprising:
a localization module configured and operable to process the measured data indicative of said data matrix and provide support recovery data indicative of the received signals originated at localized one or more subjects; and a vital signs monitoring module configured and operable to analyze the support recovery data and monitor vital signs of said localized one or more subjects.

2. The monitoring system according to claim 1, wherein said localization module is configured and operable to utilize prior knowledge of typical subject's pulse and breathing frequencies to filter said measured data and extract a subject's data matrix relating to signals received by each of said at least one receiver of the radar from the subjects in a region of interest, and apply a joint-sparse recovery processing to data indicative of said subject's data matrix utilizing sparsity in the received signals, thereby providing said support recovery data indicative of the received signals originated at localized one or more subjects.

3. The monitoring system according to claim 1, wherein said vital signs monitoring module is configured and operable to apply to said support recovery data a frequency search for the vital signs based on cardiopulmonary activities.

4. The monitoring system according to claim 1, wherein the vital signs monitoring module is configured and operable to apply a dictionary-based search for the vital signs over predetermined dictionary corresponding to frequency grids of the cardiopulmonary activities.

5. The monitoring system according to claim 2, wherein said extraction of the subject's data matrix comprises determining Doppler information in the received signals returned from the subjects in the region of interest, said Doppler information being indicative of a radial distance of each of said one or more subjects from the radar.

6. The monitoring system of claim 1, configured to localize multiple subjects at different radial distances from the FMCW radar.

7. The monitoring system of claim 1, configured to localize multiple subjects at various azimuthal angles.

8. The system of claim 1, wherein said vital signs comprise respiration rate (RR) and heartbeat rate (HR).

9. The system according to claim 1, wherein said localization module is configured and operable to carry out the following:
pre-processing the measured data acquired during acquisition time interval Tᵢₙₜ and comprising the data matrix G×L of G chirps received from the region of interest in each of L acquisition frames, L defining a slow-time dimension of the data matrix G×L, said preprocessing comprising averaging values of G chirps, to thereby obtain a corresponding N×L data matrix Y in which N defines a fast-time dimension of the matrix Y;
processing the data matrix Y and detecting the one or more subjects in the region of interest and estimating spatial locations of said one or more subjects.

10. The system according to claim 9, wherein said localization module is configured and operable to process the data matrix Y by carrying out the following:
utilizing said prior knowledge about the typical subject's pulse and breathing frequencies and performing spectral filtering of the data matrix Y along the slow-time dimension L of the data matrix Y, to thereby obtain the subject's data matrix \tilde{Y};
applying the joint-sparse recovery processing to the subject's data matrix \tilde{Y}, and obtaining a matrix \tilde{X} comprising complex amplitudes of the beat signals;
analyzing the matrix \tilde{X}, and determining the support recovery data S comprising a set of row coordinates m of matrix \tilde{X} associated with the one or more subjects in the region of interest; and
utilizing the matrix \tilde{X} and the support recovery data S and determining at least one of radial distance and azimuth angle with respect to the FMCW radar for each of M subjects, where m=1, . . . , M.

11. The monitoring system according to claim 1, comprising the frequency modulated continuous wave (FMCW) radar comprising at least one transmitter, each being configured to transmit series of millimeter wave signals to the region of interest in a clutter-rich environment, and one or more receivers associated with each of said at least one transmitter, each receiver being configured and operable to receive G chirps per acquisition frame returned from said region of interest within a field of view of the receiver, wherein the transceiver is operable to utilize, for each receiver, the single-channel front end thereof, and generate the measured data in the form of the data matrix indicative of the consecutive beat signals.

12. The monitoring system of claim 1, wherein said one or more subjects are moving subjects.

13. The system of claim 12, wherein said localization module is configured and operable to carry out the following:
pre-processing the measured data, acquired during acquisition time interval Tᵢₙₜ, by dividing said acquisition time interval Tᵢₙₜ into L time windows, wherein each time window l, l=1, . . . , L, has constant velocity of subjects' movement and comprises G frames, thereby obtaining said data matrix having a size of N×G for each chirp received from the region of interest in each of said G frames, G defining a slow-time dimension of the data matrix N×G, and N defining a fast-time dimension of the data matrix, to thereby obtain a corresponding N×G data matrix Y₁, l=1, . . . , L;
processing the data matrix Y₁ and detecting the one or more moving subjects in the region of interest and estimating spatial locations of said one or more moving subjects.

14. The system according to claim 13, wherein said localization module is configured and operable to process the data matrix Y₁ for all of said time windows l, l=1, . . . , L, by carrying out the following:
utilizing prior knowledge about typical subject's pulse and breathing frequencies and performing spectral filtering of the data matrix Y₁ along the slow-time dimension G of the data matrix Y₁, to thereby obtain a subject's data matrix \tilde{Y}ₗ;
applying joint-sparse recovery processing to the subject's data matrix \tilde{Y}ₗ, and obtaining a matrix \hat{X}ₗ comprising complex amplitudes of the consecutive beat signals;
analyzing the matrix \tilde{X}ₗ, and determining the support recovery data S[l] comprising a set of row coordinates u of the 90matrix \tilde{X}ₗ associated with U subjects in the region of interest, where u=1, . . . , U; and
utilizing the matrix \tilde{X}ₗ and the support recovery data S[l] and determining at least one of radial distance and azimuth angle with respect to the FMCW radar for each of the U subjects.

15. The monitoring system of claim 1, wherein said FMCW radar is of a single-input-multiple-output (SIMO) or multiple-input-multiple-output (MIMO) configuration implementing a time-division multiplexing (TDM), thereby allowing implementation of a uniform linear array (ULA) setup.

16. The monitoring system of claim 15, wherein said localization module is configured and operable to carry out the following:
receive as input: Tᵢₙₜ, T_{loc}, T_{win}, {y[n, k, l]}, γ, L_{f}, Iₘₐₓ, B^{(R)}, B^{(H)} wherein Tᵢₙₜ is a predefined acquisition time interval of vital signs monitoring, T_{loc} is a duration of localization corresponding to an acquisition time of first L frames within said predefined acquisition time interval Tᵢₙₜ with a frame rate fₛ, T_{win} is a duration of a preceding time window corresponding to acquisition of L frames within the predefined acquisition time interval Tᵢₙₜ, {y[n, k, l]} represents measured data for n=1, . . . , N fast-time samples, k=1, . . . K receivers, and l=1, . . . , L slow-time frames, y is a regularization parameter, L_{f} is a Lipschitz constant, Iₘₐₓ is a maximal number of iterations, B^{(R)} and B^{(H)} denote, respectively, frequency bands of respiration and heartbeat at rest;
at first T_{loc} perform the following:
assemble matrices A and B and arrange said measured data {y[n, k, l]} to construct a 3D cube {Yₗ}=ₗ₌₁^{L=T}^{_{loc}}^{f}^{ₛ} to satisfy a model Yₗ=AXₗB+Wₗ, l=1, . . . , L, where A∈ [symbol] ^{N×M} is a known range-related Vandermonde matrix, M being a number of general radial distances, B∈ [symbol] ^{P×K} is a known angle-related matrix, Xₗ∈ [symbol] ^{M×P} is an unknown matrix of complex amplitudes where Xₗ(m, p)≙\tilde{x}_{m,p}[l], P being a number of general azimuth angles, and Wₗ∈ [symbol] ^{N×K} is a noise matrix;
filter said 3D cube {Yₗ}=ₗ₌₁^{L=T}^{_{loc}}^{f}^{ₛ} utilizing said frequency bands of respiration and heartbeat at rest B^{(R)} and B^{(H)};
utilizing Radar Localization of hUmans via Joint Sparse Recovery (RaLU-JSR) method to recover {Xₗ}ₗ₌₁^{L} and the support S, defined as a the set of 2D {m, p} indices whose cardinality corresponds to a certain number Z of individuals and whose indices point to range-angle locations {d^{(z)}, θ^{(z)}}_{z=1}^{Z} of respective individuals.

17. The monitoring system of claim 16, wherein the vital signs monitoring module is configured and operable to carry out the following for each predefined acquisition time interval Tᵢₙₜ after the preceding time window T_{win}: utilize the support S to evaluate the complex amplitudes {\hat{x}_{S(z)}[l]}_{z=1}^{\hat{Z}} corresponding to vital signs of each z'th subject, and the scaled approximations of the thoracic vibrations {\hat{v}_{z}}_{z=1}^{\hat{Z}} for each z'th detected subject; and estimate the vital pairs {f_{H}^{(z)}, f_{R}^{(z)}}_{z=1}^{Z} representing, respectively, the heart and respiration rates of each z'th subject at each time interval Tᵢₙₜ, given the thoracic vibrations {\hat{v}_{z}}_{z=1}^{\hat{Z}}, and the frequency bands of respiration and heartbeat at rest B^{(R)} and B^{(H)} using an extended VSDR (E-VSDR) method.

18. The monitoring system of claim 17, wherein said vital signs monitoring module is configured and operable to perform the following:
receive as input said scaled approximations of thoracic vibrations of Z detected subjects, {\hat{v}_{z}}_{z=1}^{\hat{Z}}, and B^{(R)}, B^{(H)};
for each z'th detected subject perform the following:
for given frequency bands of respiration and heartbeat at rest B^{(R)} and B^{(H)}, express each extracted vibration \hat{v}_{z} as a linear combination of respiration and heartbeat dictionaries, D^{(R)} and D^{(H)}, respectively, each extracted vibration \hat{v}_{z} describing a frequency pattern of each z'th subject vibration;
define the respiration support of the z'th subject, [symbol] _{R}^{(z)}, as:
$${𝒮}_{R}^{( z )} = {arg max}_{q = 1 , … , {Q}_{R}} \{ \vert {D}^{( R ) T} {\hat{v}}_{z} \vert \} ,$$
and define it as the respiration rate (RR) frequency estimate, \hat{f}_{R}^{(z)} by selecting the q'th frequency within a frequency subset defined by d^{(R)};
subtract the influence of respiration by defining a residual vector, \hat{v}′_{z}, as: \hat{v}′_{z}=\hat{v}_{z}− [symbol] , where [symbol] ∈ [symbol] ^{L} is the atom of D^{(R)} corresponding to [symbol] _{R}^{(z)} and [symbol] is the estimated amplitude over [symbol] _{R}^{(z)};
mitigate the impact of interfering respiratory harmonics on heartbeat rate (HR) by estimating the respective dictionaries D^{(R′,z)} and D^{(H′,z)}, being subsets of respectively, D^{(R)} and D^{(H)}, including interfering respiration harmonics and non-interfered heart frequencies, and subtracting their contributions to define the residual vector, \hat{v}″_{z}, including the heartbeat vibration as \hat{v}″_{z}=\hat{v}′_{z}−D^{(R′,z)} â_{z}^{(R′)}, where â_{z}^{(R)} is the respective amplitude of D^{(R′,z)} describing each extracted vibration \hat{v}_{z};
estimate the heartbeat frequency of the z'th detected subject, \hat{f}_{H}^{(z)} defining it as the heartbeat support of the z'th subject defined as:
$${𝒮}_{H}^{( z )} = {arg max}_{q = 1 , … , {Q}_{{H}^{′} , z}} \{ \vert {D}^{( {H}^{′} , z ) T} {\hat{v}}_{z}^{″} \vert \} .$$

19. The monitoring system of claim 18, wherein said vital signs monitoring module is configured and operable to perform signal refinement procedure comprising the following:
for a monitoring time t, satisfying t>T_{ref}, where T_{ref} is a predetermined duration of monitoring, replace all vital estimates {\hat{f}_{H}^{(z)}, \hat{f}_{R}^{(z)}}_{z=1}^{Z} with the median value derived from the measured data collected up to the monitoring time t;
for each Tᵢₙₜ following T_{ref}:
replace the vital estimates {\hat{f}_{H}^{(z)}, \hat{f}_{R}^{(z)}}_{z=1}^{Z} with the average of the estimations acquired in the last T_{avg}^{(H)} and T_{avg}^{(R)} seconds, respectively;
replace the fixed bands of respiration and heartbeat, B^{(R)} and B^{(H)} respectively, with adaptive bands, B_{adp}^{(R)} and B_{adp}^{(H)}, centered around the vital estimates {\hat{f}_{H}^{(z)}, \hat{f}_{R}^{(z)}}_{z=1}^{Z} with small frequency margins, the adaptive bands defined in [bpm] as: B_{adp}^{(R)}(\hat{f}_{R}^{(z)})≙[\hat{f}_{R}^{(z)}−ε_{R}\hat{f}_{R}^{(z)}+ε_{R}] and B_{adp}^{(H)}(\hat{f}_{H}^{(z)})≙[\hat{f}_{H}^{(z)}−ε_{H}\hat{f}_{H}^{(z)}+ε_{H}], respectively, where ε_{R} and ε_{H} are predefined scalars which determine the margins of the corresponding bands.
